SEQUENCE LISTING (8 sequences)

SEQ ID NO: 1 - protein, 31 aa, Artificial
  synthetic polypeptide
HAEGTFTSDVSSYLEGQAAKEFIAWLVKGRG

SEQ ID NO: 2 - protein, 31 aa, Artificial
  synthetic polypeptide
HAEGTFTSDVSSYLEGQAAKEFIAWLVRGRG

SEQ ID NO: 3 - protein, 275 aa, Artificial
  synthetic polypeptide
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 4 - protein, 39 aa, Artificial
  synthetic polypeptide
HGEGTFTSDLSKQMEEEAVRLFIEWLKNGGPSSGAPPPS

SEQ ID NO: 5 - protein, 30 aa, Artificial
  synthetic polypeptide
  misc_feature (2)..(2): Xaa can be any naturally occurring amino acid
  misc_feature (29)..(29): Xaa can be any naturally occurring amino acid
HXEGTFTSDVSSYLEGQAAKEFIAWLVKXR

SEQ ID NO: 6 - protein, 44 aa, Artificial
  synthetic polypeptide
  misc_feature (5)..(5): Xaa can be any naturally occurring amino acid
  misc_feature (7)..(7): Xaa can be any naturally occurring amino acid
  misc_feature (23)..(23): Xaa can be any naturally occurring amino acid
HGEGXFXSDLSKQMEEEAVRLFXEWLKNGGPSSGAPPSKKKKKK

SEQ ID NO: 7 - protein, 645 aa, Artificial
  synthetic polypeptide
HGEGTFTSDVSSYLEGQAAKEFIAWLVKGRHGEGTFTSDVSSYLEGQAAKEFIAWLVKGRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 8 - protein, 29 aa, Artificial
  synthetic polypeptide
EGTFTSDVSSYLEGQAAKEFIAWLVRGRG

(19) United States
(12) Patent Application Publication
(10) Pub. No.: US 2022/0143003 A1
(43) Pub. Date: May 12, 2022

(54) COMPOSITIONS AND METHODS TO TREAT NON-ALCOHOLIC FATTY LIVER DISEASES (NAFLD)

(71) Applicant: Coherus Biosciences, Inc., Redwood City, CA (US)

(72) Inventors: Christos Mantzoros, Watertown, MA (US); Glenn D. Rosen, Los Altos, CA (US)

(21) Appl. No.: 17/419,559

(22) PCT Filed: Dec. 27, 2019
(86) PCT No.: PCT/US2019/068710

§ 371 Date: Jun. 29, 2021

Related U.S. Application Data
(60) Provisional application No. 62/827,349, filed on Apr. 1, 2019.
(60) Provisional application No. 62/786,618, filed on Dec. 31, 2018.

(51) Int. Cl. A61K 31/47; A61K 45/06; A61P 1/16

(52) U.S. Cl. CPC A61K 31/47; A61P 1/16; A61K 45/06

(57) ABSTRACT

Provided herein are methods and combination therapies useful for the treatment of non-alcoholic fatty liver diseases (NAFLD). In particular, provided herein are methods and combination therapies for treating NAFLD by administering a combination therapy comprising (a) the compound of Formula (I), or a pharmaceutically acceptable salt or solvate thereof, and (b) an SGLT-2 inhibitor, or a pharmaceutically acceptable salt or solvate thereof, and/or (c) a GLP-1 receptor agonist, or a pharmaceutically acceptable salt or solvate thereof, and/or (d) metformin. Also provided are pharmaceutical compositions and pharmaceutical combinations comprising the compound of Formula (I) and an SGLT-2 inhibitor and/or a GLP-1 receptor agonist and/or metformin.

### CROSS REFERENCE TO RELATED APPLICATIONS

This application claims priority to U.S. Provisional Application Ser. No. 62/786,618, filed on Dec. 31, 2018, and 62/827,349, filed on Apr. 1, 2019, each of which is herein incorporated by reference in its entirety.

### TECHNICAL FIELD

The present disclosure relates to methods and combination therapies useful for the treatment of non-alcoholic fatty liver diseases (NAFLD). In particular, this disclosure relates to methods and combination therapies for treating NAFLD by administering a combination therapy comprising a PPARγ inhibitor that is the compound of Formula (I), or a pharmaceutically acceptable salt or solvate thereof, and an SGLT-2 inhibitor, or a pharmaceutically acceptable salt or solvate thereof, and/or a GLP-1 receptor agonist, or a pharmaceutically acceptable salt or solvate thereof; and/or metformin, or a pharmaceutically acceptable salt thereof.

### BACKGROUND

Non-alcoholic fatty liver disease (NAFLD) is characterized by the presence of hepatic fat accumulation in the absence of secondary causes of hepatic steatosis including excessive alcohol consumption, other known liver diseases, or long-term use of a steatogenic medication (Perumpail et al., World J Gastroenterol. 2017, 23(47):8263-8438 and Chalasani et al., Hepatology. 2018, 67(1):328-357). NAFLD encompasses two categories: simple non-alcoholic fatty liver (NAFL) and non-alcoholic steatohepatitis (NASH). Typically, NAFL has a more indolent course of progression whereas NASH is a more severe form associated with inflammation that may progress more rapidly to end-stage liver disease. NAFL and/or NASH may also include scarring of the liver known as liver fibrosis or in a more severe form, liver cirrhosis. Scarring of the liver reduces liver function up to and including liver failure.

NAFLD is currently the most common liver disease in the world (Perumpail et al., World J Gastroenterol. 2017, 23(47):8263-8438) with approximately one-fourth of the adult population suffering from NAFLD worldwide (Sumida, et al., J Gastroenterol. 2018, 53:362-376). There are many risk factors associated with NAFLD including hypertension, obesity, diabetes, and hyperlipidemia with a particularly close association with type II diabetes mellitus and NAFLD (Vernon et al., Aliment Pharmacol Ther. 2011, 34:274-285).

Lifestyle interventions including dietary caloric restriction and exercise are the most effective methods of prevention and treatment for NAFLD (Sumida, et al., J Gastroenterol. 2018, 53:362-376). However, these can be difficult treatments to follow. Thus, there is a need for pharmaceuticals to treat NAFLD. Current pharmaceutical treatments that have been proposed or tested in prior trials, although are not yet approved for NAFLD include vitamin E, ω3 fatty acid, statin, metformin, orlistat, thiazolidinediones (“TZDs”), urodeoxycholic acid, pioglitazone, and pentoxifilline (Sumida, et al., J Gastroenterol. 2018, 53:362-376). However, there is currently no approved pharmacotherapy for NAFLD.

### SUMMARY

Provided herein in some embodiments is a method of treating non-alcoholic fatty liver disease (NAFLD) in a subject in need thereof comprising administering to the subject
(a) the compound of Formula (I),

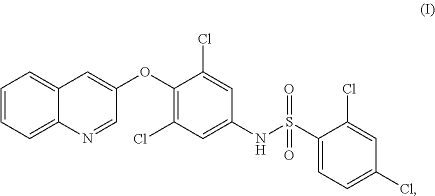 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) an SGLT inhibitor, or a pharmaceutically acceptable salt or solvate thereof, wherein the amounts of (a) and (b) together are effective in treating NAFLD.

Provided herein in some embodiments is a method of treating non-alcoholic fatty liver disease (NAFLD) in a subject in need thereof comprising administering to the subject
(a) the compound of Formula (I),

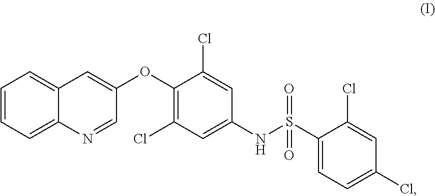 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) an SGLT-2 inhibitor, or a pharmaceutically acceptable salt or solvate thereof, wherein the amounts of (a) and (b) together are effective in treating NAFLD.
Provided herein in some embodiments is a method of treating a subject, the method comprising:
selecting a subject having non-alcoholic fatty liver disease (NAFLD); and
administering
(a) the compound of Formula (I),

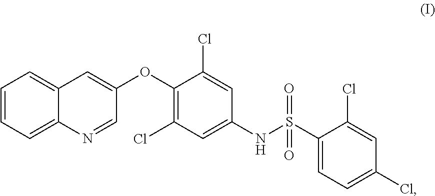 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) an SGLT inhibitor, or a pharmaceutically acceptable salt or solvate thereof, to the selected subject,
   wherein the amounts of (a) and (b) together are effective in treating NAFLD.
Provided herein in some embodiments is a method of treating a subject, the method comprising:
identifying a subject having non-alcoholic fatty liver disease (NAFLD); and
administering
(a) the compound of Formula (I),

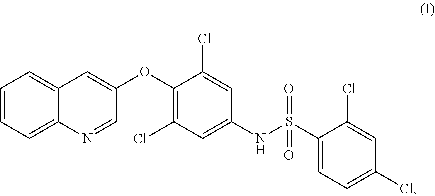 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) an SGLT inhibitor, or a pharmaceutically acceptable salt or solvate thereof, to the selected subject,
   wherein the amounts of (a) and (b) together are effective in treating NAFLD.

Provided herein in some embodiments is a method of treating non-alcoholic fatty liver disease (NAFLD) in a subject in need thereof comprising administering to the subject
(a) a therapeutically effective amount of the compound of Formula (I),

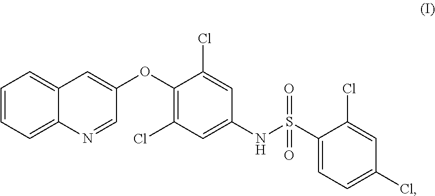 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) a therapeutically effective amount of an SGLT inhibitor, or a pharmaceutically acceptable salt or solvate thereof.

Provided herein in some embodiments is a method of treating a subject, the method comprising:
selecting a subject having non-alcoholic fatty liver disease (NAFLD); and
administering
(a) a therapeutically effective amount of the compound of Formula (I),

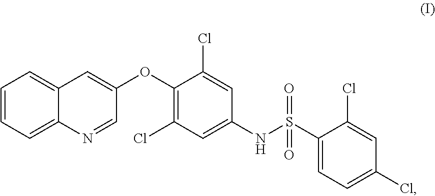 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) a therapeutically effective amount of an SGLT inhibitor, or a pharmaceutically acceptable salt or solvate thereof, to the selected subject.

Provided herein in some embodiments is a method of treating fibrosis in a subject in need thereof comprising administering to the subject
(a) the compound of Formula (I),

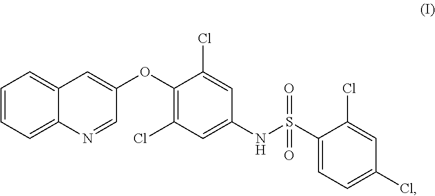 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) an SGLT inhibitor, or a pharmaceutically acceptable salt or solvate thereof, wherein the amounts of (a) and (b) together are effective in treating NAFLD.

Provided herein in some embodiments is a method of treating fibrosis in a subject in need thereof comprising administering to the subject
(a) a therapeutically effective amount of the compound of Formula (I),

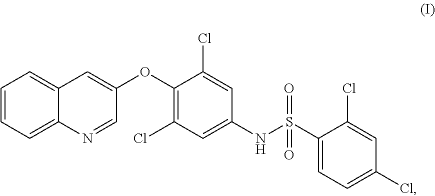 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) a therapeutically effective amount of an SGLT inhibitor, or a pharmaceutically acceptable salt or solvate thereof.

Provided herein in some embodiments is a method of treating a subject, the method comprising:
selecting a subject having non-alcoholic fatty liver disease (NAFLD); and
administering
(a) the compound of Formula (I),

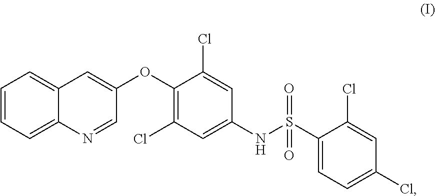 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) an SGLT-2 inhibitor, or a pharmaceutically acceptable salt or solvate thereof, to the selected subject,
   wherein the amounts of (a) and (b) together are effective in treating NAFLD.
   Provided herein in some embodiments is a method of treating a subject, the method comprising:
   identifying a subject having non-alcoholic fatty liver disease (NAFLD); and
   administering
(c) the compound of Formula (I),

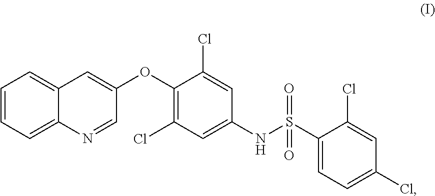 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(d) an SGLT-2 inhibitor, or a pharmaceutically acceptable salt or solvate thereof, to the selected subject,
   wherein the amounts of (a) and (b) together are effective in treating NAFLD.

Provided herein in some embodiments is a method of treating non-alcoholic fatty liver disease (NAFLD) in a subject in need thereof comprising administering to the subject
(c) a therapeutically effective amount of the compound of Formula (I),

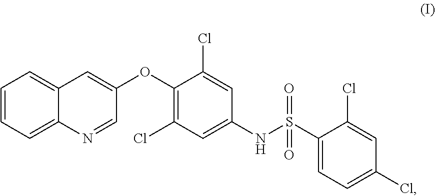 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(d) a therapeutically effective amount of an SGLT-2 inhibitor, or a pharmaceutically acceptable salt or solvate thereof.

Provided herein in some embodiments is a method of treating a subject, the method comprising:
selecting a subject having non-alcoholic fatty liver disease (NAFLD); and
administering
(c) a therapeutically effective amount of the compound of Formula (I),

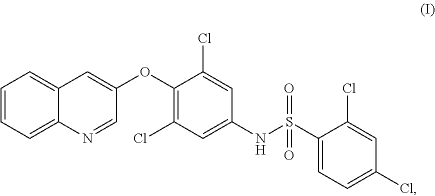 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(d) a therapeutically effective amount of an SGLT-2 inhibitor, or a pharmaceutically acceptable salt or solvate thereof, to the selected subject.

Provided herein in some embodiments is a method of treating fibrosis in a subject in need thereof comprising administering to the subject
(c) the compound of Formula (I),

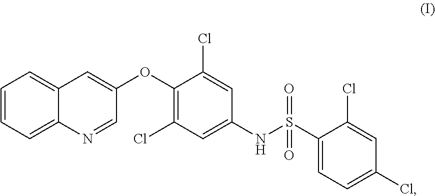 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(d) an SGLT-2 inhibitor, or a pharmaceutically acceptable salt or solvate thereof, wherein the amounts of (a) and (b) together are effective in treating NAFLD.

Provided herein in some embodiments is a method of treating fibrosis in a subject in need thereof comprising administering to the subject
(c) a therapeutically effective amount of the compound of Formula (I),

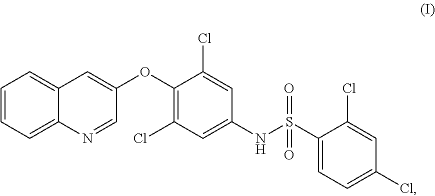 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(d) a therapeutically effective amount of an SGLT-2 inhibitor, or a pharmaceutically acceptable salt or solvate thereof.

In some more particular embodiments, (a) and (b) are administered concurrently.

In some more particular embodiments, (a) and (b) are administered sequentially in either order.

In some more particular embodiments, the method further comprises administering (c) a GLP-1 receptor agonist, or a pharmaceutically acceptable salt thereof, and/or (d) metformin, or a pharmaceutically acceptable salt thereof, for example, in some more particular embodiments, the method further comprises administering (c) a GLP-1 receptor agonist, or a pharmaceutically acceptable salt thereof. In some other more particular embodiments, the method further comprises administering (d) metformin, or a pharmaceutically acceptable salt thereof. In still other more particular embodiments, the method further comprises administering (c) a GLP-1 receptor agonist, or a pharmaceutically acceptable salt thereof, and (d) metformin, or a pharmaceutically acceptable salt thereof.

Provided herein in some embodiments is a method of treating non-alcoholic fatty liver disease (NAFLD) in a subject in need thereof comprising administering to the subject
(a) the compound of Formula (I),

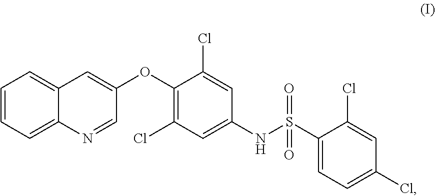 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) a GLP-1 receptor agonist, or a pharmaceutically acceptable salt or solvate thereof, wherein the amounts of (a) and (b) together are effective in treating NAFLD.
   Provided herein in some embodiments is a method of treating a subject, the method comprising:
   selecting a subject having non-alcoholic fatty liver disease (NAFLD); and
   administering
(a) the compound of Formula (I),

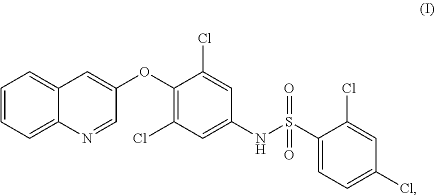 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) a GLP-1 receptor agonist, or a pharmaceutically acceptable salt or solvate thereof, to the selected subject
   wherein the amounts of (a) and (b) together are effective in treating NAFLD.
   Provided herein in some embodiments is a method of treating a subject, the method comprising:
   identifying a subject having non-alcoholic fatty liver disease (NAFLD); and
   administering
   (a) the compound of Formula (I),

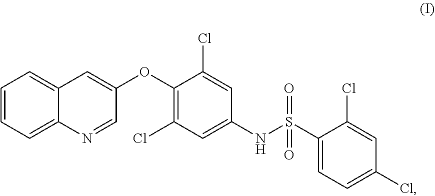 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) a GLP-1 receptor agonist, or a pharmaceutically acceptable salt or solvate thereof, to the selected subject
wherein the amounts of (a) and (b) together are effective in treating NAFLD.

Provided herein in some embodiments is a method of treating non-alcoholic fatty liver disease (NAFLD) in a subject in need thereof comprising administering to the subject
(a) a therapeutically effective amount of the compound of Formula (I),

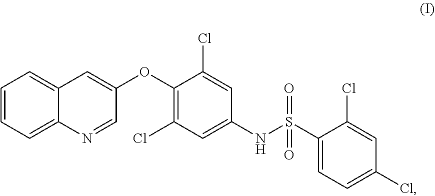 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) a therapeutically effective amount of a GLP-1 receptor agonist, or a pharmaceutically acceptable salt or solvate thereof.

Provided herein in some embodiments is a method of treating a subject, the method comprising:
selecting a subject having non-alcoholic fatty liver disease (NAFLD); and
administering
(a) a therapeutically effective amount of the compound of Formula (I),

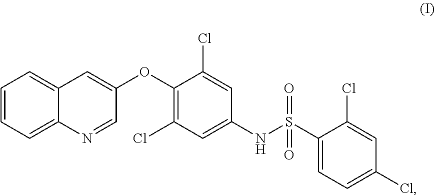 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) a therapeutically effective amount of a GLP-1 receptor agonist, or a pharmaceutically acceptable salt or solvate thereof, to the selected subject.

Provided herein in some embodiments is a method of treating fibrosis in a subject in need thereof comprising administering to the subject
(a) the compound of Formula (I),

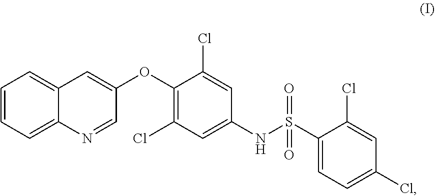 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(b) a GLP-1 receptor agonist, or a pharmaceutically acceptable salt or solvate thereof, wherein the amounts of (a) and (b) together are effective in treating fibrosis.

Provided herein in some embodiments is a method of treating fibrosis in a subject in need thereof comprising administering to the subject
(c) a therapeutically effective amount of the compound of Formula (I),

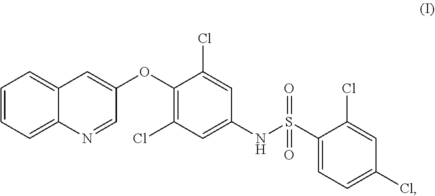 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, and
(d) a therapeutically effective amount of a GLP-1 receptor agonist, or a pharmaceutically acceptable salt or solvate thereof.

In some more particular embodiments, (a) and (b) are administered concurrently.

In some more particular embodiments, (a) and (b) are administered sequentially in either order.

In some more particular embodiments, the method further comprises administering (c) a SGLT inhibitor, or a pharmaceutically acceptable salt or solvate thereof, and/or (d) metformin, or a pharmaceutically acceptable salt thereof, for example, in some more particular embodiments, the method further comprises administering (c) an SGLT inhibitor, or a pharmaceutically acceptable salt or solvate thereof, or the method further comprises administering (d) metformin, or a pharmaceutically acceptable salt thereof. In other more particular embodiments, the method further comprises administering (c) a SGLT inhibitor, or a pharmaceutically acceptable salt or solvate thereof, and (d) metformin, or a pharmaceutically acceptable salt thereof.

In some more particular embodiments, the method further comprises administering (c) a SGLT-2 inhibitor, or a pharmaceutically acceptable salt or solvate thereof, and/or (d) metformin, or a pharmaceutically acceptable salt thereof, for example, in some more particular embodiments, the method further comprises administering (c) an SGLT-2 inhibitor, or a pharmaceutically acceptable salt or solvate thereof, or the method further comprises administering (d) metformin, or a pharmaceutically acceptable salt thereof. In other more particular embodiments, the method further comprises administering (c) a SGLT-2 inhibitor, or a pharmaceutically acceptable salt or solvate thereof, and (d) metformin, or a pharmaceutically acceptable salt thereof.

Provided herein in some embodiments is a pharmaceutical composition comprising
(a) the compound of Formula (I),

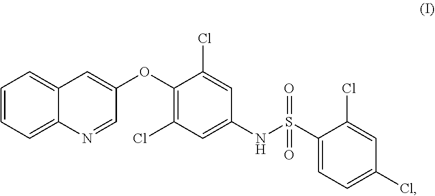 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof,
(b) an SGLT inhibitor, or a pharmaceutically acceptable salt or solvate thereof, and
one or more pharmaceutical excipients, wherein the amounts of (a) and (b) together are effective in treating NAFLD.
Provided herein in some embodiments is a pharmaceutical composition comprising
(a) the compound of Formula (I),

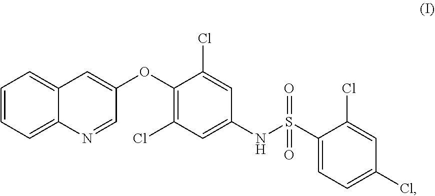 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof,
(b) an SGLT-2 inhibitor, or a pharmaceutically acceptable salt or solvate thereof, and
one or more pharmaceutical excipients, wherein the amounts of (a) and (b) together are effective in treating NAFLD.
Provided herein in some embodiments is a pharmaceutical composition comprising
(a) the compound of Formula (I),

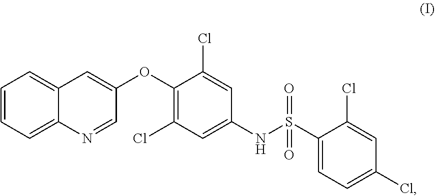 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof,
(b) a GLP-1 receptor agonist, or a pharmaceutically acceptable salt or solvate thereof, and
one or more pharmaceutical excipients, wherein the amounts of (a) and (b) together are effective in treating NAFLD.
Provided herein in some embodiments is a pharmaceutical composition comprising
(a) the compound of Formula (I),

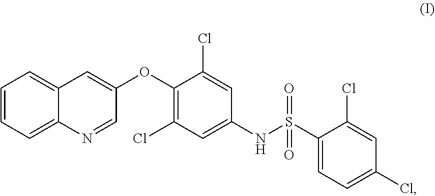 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof,
(b) an SGLT-2 inhibitor, or a pharmaceutically acceptable salt or solvate thereof,
(c) a GLP-1 receptor agonist, or a pharmaceutically acceptable salt or solvate thereof, and
one or more pharmaceutical excipients, wherein the amounts of (a) and (b) and (c) together are effective in treating NAFLD.
Provided herein in some embodiments is a pharmaceutical composition comprising
(a) the compound of Formula (I),

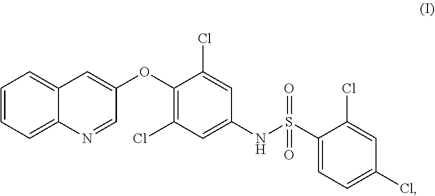 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof,
(b) an SGLT-2 inhibitor, or a pharmaceutically acceptable salt or solvate thereof,
(c) metformin, or a pharmaceutically acceptable salt thereof, and
one or more pharmaceutical excipients, wherein the amounts of (a) and (b) and (c) together are effective in treating NAFLD.
Provided herein in some embodiments is a pharmaceutical composition comprising
(a) the compound of Formula (I),

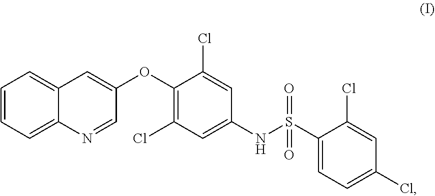 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof,
(b) a GLP-1 receptor agonist, or a pharmaceutically acceptable salt or solvate thereof,
(c) metformin, or a pharmaceutically acceptable salt thereof, and
one or more pharmaceutical excipients, wherein the amounts of (a) and (b) and (c) together are effective in treating NAFLD.
Provided herein in some embodiments is a pharmaceutical composition comprising
(a) the compound of Formula (I),

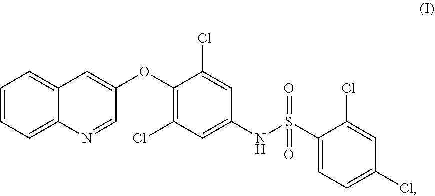 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof,
(b) an SGLT-2 inhibitor, or a pharmaceutically acceptable salt or solvate thereof,
(c) a GLP-1 receptor agonist, or a pharmaceutically acceptable salt or solvate thereof,
(d) metformin, or a pharmaceutically acceptable salt thereof, and
one or more pharmaceutical excipients, wherein the amounts of (a) and (b) and (c) and (d) together are effective in treating NAFLD.
In some embodiments, the NAFLD is non-alcoholic steatohepatitis (NASH).
In some embodiments, the fibrosis is hepatic fibrosis.
In some embodiments, the compound of formula (I), or a pharmaceutically acceptable salt or solvate thereof, is provided as a pharmaceutically acceptable salt. In some embodiments, the SGLT-2 inhibitor, or a pharmaceutically acceptable salt or solvate thereof, is an SGLT-2 inhibitor provided as a pharmaceutically acceptable solvate. In other embodiments, the SGLT-2 inhibitor, or a pharmaceutically acceptable salt or solvate thereof, is an SGLT-2 inhibitor provided as a pharmaceutically acceptable salt. In some embodiments, the metformin, or a pharmaceutically acceptable salt thereof, is provided as the metformin free base. In some embodiments, the GLP-1 receptor agonist, or a pharmaceutically acceptable salt or solvate thereof, is provided as the GLP-1 receptor agonist free base.

In some embodiments of the pharmaceutical compositions provided herein, the pharmaceutical compositions comprise at least one pharmaceutically acceptable carrier.

In some more particular embodiments, a method as provided herein comprises administering a pharmaceutical composition as provided herein to a subject twice a day, daily, every other day, three times a week, twice a week, weekly, every other week, twice a month, or monthly.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE FIGURES

FIG. 1 illustrates the absolute body weight in DIO-NASH mice at the termination of the study described in Example 2.

FIG. 2 illustrates the relative body weight in DIO-NASH mice at the termination of the study described in Example 2.

FIG. 3 illustrates the discrete daily food intake during weeks 1-2 of the study described in Example 2.

FIG. 4 illustrates the cumulative daily food intake during weeks 1-2 of the study described in Example 2.

FIG. 5 illustrates the weekly food intake during week 3-12 of the study described in Example 2.

FIG. 6 provides a summary of fibrosis stage of histopathological scoring of pre- and post-study biopsies. For each group, the number of animals with a higher (worsening), same or lower (improvement) in score at post- compared to pre-study is indicated by the height of the bar. More animals with a lower score in a treatment group indicates improvement.

FIG. 7 provides a summary of NAFLD activity score of histopathological scoring of pre- and post-study biopsies. For each group, the number of animals with a higher (worsening), same or lower (improvement) in score at post- compared to pre-study is indicated by the height of the bar. More animals with a lower score in a treatment group indicates improvement.

FIG. 8 provides a summary of NAFLD activity score showing individual scores for steatosis of pre- and post-study biopsies. For each group the number of animals with a higher (worsening), same or lower (improvement) in score at post- compared to pre-study is indicated by the height of the bar. More animals with a lower score in a treatment group indicates improvement.

FIG. 9 provides a summary of NAFLD activity score showing individual scores for lobular inflammation of pre- and post-study biopsies. For each group the number of animals with a higher (worsening), same or lower (improvement) in score at post-compared to pre-study is indicated by the height of the bar. More animals with a lower score in a treatment group indicates improvement.

FIG. 10 provides a summary of NAFLD activity score showing individual scores for ballooning degeneration of pre- and post-study biopsies. For each group the number of animals with a higher (worsening), same or lower (improvement) in score at post-compared to pre-study is indicated by the height of the bar. More animals with a lower score in a treatment group indicates improvement.

FIG. 11 provides an outline of a study to assess the effects of treatment with CHS-131 (Compound of Formula (I)), alone and in combination with other therapeutic agents, to treat NASH, as described in Example 3.

### DETAILED DESCRIPTION

### Definitions

Reference to the term “about” has its usual meaning in the context of pharmaceutical compositions to allow for reasonable variations in amounts that can achieve the same effect and also refers herein to a value of plus or minus 10% of the provided value. For example, “about 20” means or includes amounts from 18 to and including 22.

The term “administration” or “administering” refers to a method of giving a dosage of a compound or pharmaceutical composition to a vertebrate or invertebrate, including a mammal, a bird, a fish, or an amphibian. The preferred method of administration can vary depending on various factors, e.g., the components of the pharmaceutical composition, the site of the disease, and the severity of the disease.

The term “CHS-131” as used herein refers to a compound of Formula (I):

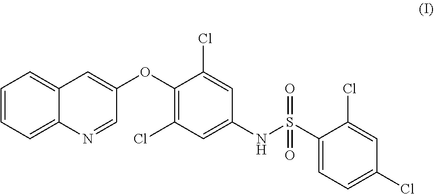 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof.
The compound of Formula (I) is a selective peroxisome proliferator-activated receptor (PPAR) 7 modulator. The compound of Formula (I) is disclosed in, for example, U.S. Pat. Nos. 7,041,691; 6,200,995; 6,583,157; 6,653,332; and U.S. Publication Application No. 2016/0260398, the contents of each of which are incorporated by reference herein in their entireties.

The compound of Formula (I) can be prepared, for example, by the methods described in U.S. Pat. No. 6,583,157 or 6,200,995, each of which is incorporated by reference in its entirety herein. In some embodiments, different salts, e.g., besylate, tosylate HCl, or HBr salts, and/or polymorphs of the compound of Formula (I) are used within the methods and compositions described herein. Salts and polymorphs of the compound of Formula (I), such as those provided herein, can be prepared according to the methods described in U.S. Pat. Nos. 6,583,157 and 7,223,761, the contents of each of which are incorporated by reference in their entireties.

The term “SGLT inhibitor” as used herein refers to a compound that inhibits one or more Sodium Glucose Co-Transporters. In one embodiment, an SLGT inhibitor is a compound that inhibits the Sodium Glucose Co-Transporter-1 (SGLT-1). In another embodiment, an SLGT inhibitor is a compound that inhibits the Sodium Glucose Co-Transporter-2 (SGLT-2). In yet another embodiment, an SLGT inhibitor is a compound that inhibits both SGLT-1 and SGLT-2.

The term “SGLT-1 inhibitor” as used herein refers to a compound that inhibits the Sodium Glucose Co-Transporter-1 (SGLT-1). SGLT-1 primarily absorbs glucose in the small intestine and also reabsorbs glucose in the kidneys. By disrupting these functions, SGLT-1 inhibitors exert a glucose-lowering effect. See, Spatola et al., Diabetes Ther. 2017; 9(1):427-430. The term “SGLT-1 inhibitor” is not limited to compounds that only inhibit SGLT-1, thus includes compounds that have other activities in addition to SGLT-1 inhibition. Examples of SGLT-1 inhibitors include, but are not limited to, LX2761 (Lexicon Pharmaceuticals; See, Powell et al., J Pharmacol Exp Ther. 2017 July; 362(1):85-97), licofliglozin and sotagliflozin (ZYNQUISTA™).

The term “SGLT-2 inhibitor” as used herein refers to a compound that inhibits the Sodium Glucose Co-Transporter-2 (SGLT-2). SGLT-2 inhibitors disrupt reabsorption of glucose by the kidneys and thus exert a glucose-lowering effect. By enhancing glucosuria, independently of insulin, SLGT-2 inhibitors have been shown to treat type 2 diabetes and improve cardiovascular outcomes. See, Wright, 2001, Am J Physiol Renal Physiol 280:F10; and Scheen, 2018, Circ Res 122:1439. SGLT2 inhibitors include a class of drugs known as gliflozins. The term “SGLT-2 inhibitor” is not limited to compounds that only inhibit SGLT-2, thus includes compounds that have other activities in addition to SGLT-2 inhibition. Examples of SGLT-2 inhibitors include, but are not limited to, bexagliflozin, canagliflozin (INVOKANA®), dapagliflozin (FARXIGA®), empagliflozin (JARDIANCE®), ertugliflozin (STEGLATRO™), ipragliflozin (SUGLAT®), luseogliflozin (LUSEFI®), remogliflozin, serfliflozin, licofliglozin, sotagliflozin (ZYNQUISTA™), and tofogliflozin.

The term “SGLT-1/2 dual inhibitor” and “SGLT dual inhibitor” as used herein refers to a compound that inhibits both SGLT-1 and SGLT-2. See, Danne, et al., Diabetes Technol Ther. 2018 June; 20(S2):5269-5277. Examples of dual inhibitors include, but are not limited to, licofliglozin and sotagliflozin (ZYNQUISTA™).

The term “GLP-1 receptor agonist” or “GLP-1 RA” as used herein refers to an agonist of the Glucagon-like peptide-1 (GLP-1) receptor. GLP-1 RAs enhance glucose-dependent insulin secretion, suppress inappropriately elevated glucagon levels, both in fasting and postprandial states, and slow gastric emptying. Karla et al., Glucagon-like peptide-1 receptor agonists in the treatment of type 2 diabetes: Past, present, and future, Indian J Endocrinol Metab. 2016 March-April; 20(2): 254-267. GLP-1 RAs have been shown to treat type 2 diabetes. Examples of GLP-1 RAs include, but are not limited to, albiglutide, dulaglutide, efpeglenatide, exenatide, liraglutide, lixisenatide, semaglutide, and tirzepatide.

GLP-1 receptor agonists include analogs of native GLP-1 (see, e.g., the native GLP-1 (7-37) amino acid sequence below) and peptides based on exendin, which is a peptide derived from the Gila monster. Non-limiting examples of GLP-1 receptor agonists include liraglutide (VICTOZA®, NN2211), dulaglutide (LY2189265, TRULICITY®), exenatide (BYETTA®, BYDUREON®, Exendin-4), taspoglutide, lixisenatide (LYXUMIA®), albiglutide (TANZEUM®), semaglutide (OZEMPIC®), ZP2929, NNC0113-0987, BPI-3016, and TT401. Non-limiting examples of analogs of native GLP-1 include liraglutide and semaglutide. Non-limiting examples of GLP-1 receptor agonists based on exendin include exanatide and lixisenatide. In some embodiments, the GLP-1 receptor agonist is a compound having 90% or greater sequence identity to any of the GLP-1 receptor agonists described herein, e.g., the sequences of the GLP-1 receptor agonists as shown in Table 1. For example, at least 90%, 91%, 92%, 93%, 94%, 95%, 96%, 97%, or 99% or greater sequence identity. In some embodiments, the GLP-1 receptor agonistis a compound having at least 90% or greater sequence identity to any of the GLP-1 receptor agonists described herein and at least 80% of the activity, for example, as determined by cyclic adenosine monophosphate (cAMP) response element (CRE)-luciferase based reporter-gene assays, cAMP-responsive CRE4-luciferase assay, or cAMP-responsive CRE-BLAM reporter assays (e.g., those described in Sai et al. Int J Mol Sci. 2017 March; 18(3): 578 and Glaesner et al., Diabetes Metab Res Rev. 2010 May; 26(4):287-96). For example, at least 90%, 91%, 92%, 93%, 94%, 95%, 96%, 97%, or 99% or greater sequence identity and at least 80%, 85%, 90%, 95%, or 99% of the activity.

TABLE 1
Sequence and modifications of GLP-1 receptor agonists

| GLP-1 Receptor | |
|---|---|
| Agonist | Sequences and Modifications |
| GLP-1 (7-37) | HAEGTFTSDV SSYLEGQAAK EFIAWLVKGR G (SEQ ID |
| | NO: 1) |
| Liraglutide | HAEGTFTSDV SSYLEGQAAK(γ-Glu-palmitoyl) EFIAWLVRGR |
| | G (SEQ ID NO: 2) |
| Dulaglutide | HGEGTFTSDV SSYLEEQAAK EFIAWLVKGG GGGGGSGGGG |
| | SGGGGSAESK YGPPCPPCPA PEAAGGPSVF LFPPKPKDTL |
| | MISRTPEVTC VVVDVSQEDP EVQFNWYVDG VEVHNAKTKP |
| | REEQFNSTYR VVSVLTVLHQ DWLNGKEYKC KVSNKGLPSS |
| | IEKTISKAKG QPREPQVYTL PPSQEEMTKN QVSLTCLVKG |
| | FYPSDIAVEW ESNGQPENNY KTTPPVLDSD GSFFLYSRLT |
| | VDKSRWQEGN VFSCSVMHEA LHNHYTQKSL SLSLG (SEQ |
| | ID NO: 3) |
| | In some embodiments, dulaglutide is a dimer of the above |
| | sequence with disulfide bridges between Cys90-Cys150 and |
| | Cys196-Cys254 of each monomer and between Cys55-Cys55 and |
| | Cys58-Cys58 of the dimers. |
| Exenatide | HGEGTFTSDL SKQMEEEAVR LFIEWLKNGG PSSGAPPPS |
| | (SEQ ID NO: 4) |
| Taspoglutide | HXEGTFTSDV SSYLEGQAAK EFIAWLVKXR (SEQ ID |
| | NO: 5) |
| Lixisenatide | HGEGXFXSDL SKQMEEEAVR LFXEWLKNGG PSSGAPPSKK |
| | KKKK (SEQ ID NO: 6) |
| Albiglutide | HGEGTFTSDV SSYLEGQAAK EFIAWLVKGR HGEGTFTSDV |
| | SSYLEGQAAK EFIAWLVKGR DAHKSEVAHR FKDLGEENFK |
| | ALVLIAFAQY LQQCPFEDHV KLVNEVTEFA KTCVADESAE |
| | NCDKSLHTLF GDKLCTVATL RETYGEMADC CAKQEPERNE |
| | CFLQHKDDNP NLPRLVRPEV DVMCTAFHDN EETFLKKYLY |
| | EIARRHPYFY APELLFFAKR YKAAFTECCQ AADKAACLLP |
| | KLDELRDEGK ASSAKQRLKC ASLQKFGERA FKAWAVARLS |
| | QRFPKAEFAE VSKLVTDLTK VHTECCHGDL LECADDRADL |
| | AKYICENQDS ISSKLKECCE KPLLEKSHCI AEVENDEMPA |
| | DLPSLAADFV ESKDVCKNYA EAKDVFLGMF LYEYARRHPD |
| | YSVVLLLRLA KTYETTLEKC CAAADPHECY AKVFDEFKPL |
| | VEEPQNLIKQ NCELFEQLGE YKFQNALLVR YTKKVPQVST |
| | PTLVEVSRNL GKVGSKCCKH PEAKRMPCAE DYLSVVLNQL |
| | CVLHEKTPVS DRVTKCCTES LVNRRPCFSA LEVDETYVPK |
| | EFNAETFTFH ADICTLSEKE RQIKKQTALV ELVKHKPKAT |
| | KEQLKAVMDD FAAFVEKCCK ADDKETCFAE EGKKLVAASQ |
| | AALGL (SEQ ID NO: 7) |
| | Albiglutide is a peptide with the above sequence (which |
| | comprises a dimer of modified GLP-1 fused to human albumin). |
| | In some embodiments, abliglutide also has disulfide bridges |
| | linking amino acids 113-122, 135-151, 150-161, 184-229, 228- |
| | 237, 260-306, 305-313, 325-339, 338-349, 376-421, 420-429, |
| | 452-498, 497-508, 521-537, 536-547, 574-619, and 618-627. |
| Semaglutide | H-Aib-EGTFTSDV SSYLEGQAAK (AEEAc-AEEAc-γ-Glu- |
| | 17-carboxyheptadecanoyl) EFIAWLVRGR G (SEQ ID |
| | NO: 8) |

“Metformin” refers to the compound N,N-dimethylimidodicarbonimidic diamide, shown below.

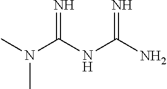

By “effective dosage” or “therapeutically effective amount” or “pharmaceutically effective amount” of a compound as provided herein is an amount that is sufficient to achieve the desired therapeutic effect and can vary according to the nature and severity of the disease condition, and the potency of the compound. A therapeutic effect is the relief, to some extent, of one or more of the symptoms of the disease, and can include curing a disease. “Curing” means that the symptoms of active disease are eliminated. However, certain long-term or permanent effects of the disease can exist even after a cure is obtained (such as, e.g., extensive tissue damage). In some embodiments, a “therapeutically effective amount” of a compound as provided herein refers to an amount of the compound that is effective as a monotherapy. In some embodiments, the therapeutic effect is determined from one or more parameters selected from the NAFLD Activity Score (NAS), hepatic steatosis, hepatic inflammation, biomarkers indicative of liver damage, and liver fibrosis and/or liver cirrhosis. For example, a therapeutic effect can include one or more of a decrease in symptoms, a decrease in the NAS, a reduction in the amount of hepatic steatosis, a decrease in hepatic inflammation, a decrease in the level of biomarkers indicative of liver damage, and a reduction in liver fibrosis and/or liver cirrhosis, a lack of further progression of liver fibrosis and/or liver cirrhosis, or a slowing of the progression of liver fibrosis and/or liver cirrhosis following administration of a compound or compounds as described herein.

In some embodiments, the amounts of the two or more compounds as provided herein together are effective in treating NAFLD (e.g., the amounts of the compound of Formula (I) and an SGLT-2 inhibitor or GLP-1 receptor agonist together are effective in treating NAFLD). In such embodiments, the amount of each agent is also referred to as a “jointly therapeutically effective amount.” In some embodiments, the amounts of the two or more compounds as provided herein together are effective in treating NAFLD (e.g., the amounts of the compound of Formula (I) and an SGLT-2 inhibitor and/or GLP-1 receptor agonist and/or metformin, together are effective in treating NAFLD). In such embodiments, the amount of each agent is also referred to as a “jointly therapeutically effective amount.” For example, the therapeutic agents of a combination described herein are given to the patient simultaneously or separately (e.g., in a chronologically staggered manner, for example a sequence-specific manner) in such time intervals that they show an interaction (e.g., a joint therapeutic effect). For example, wherein the amounts of (a) the compound of Formula (I), or a pharmaceutically acceptable salt or solvate thereof, and (b) an SGLT-2 inhibitor, or a pharmaceutically acceptable salt or solvate thereof, or a GLP-1 receptor agonist, or a pharmaceutically acceptable salt or solvate thereof, together are effective in treating NAFLD, the joint therapeutic effect of the combination of (a) the compound of Formula (I), or a pharmaceutically acceptable salt or solvate thereof, and (b) an SGLT-2 inhibitor, or a pharmaceutically acceptable salt or solvate thereof, or a GLP-1 receptor agonist, or a pharmaceutically acceptable salt or solvate thereof, is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of the compound of Formula (I), or a pharmaceutically acceptable salt or solvate thereof alone.

In some embodiments, the amounts of (a) the compound of Formula (I), or a pharmaceutically acceptable salt or solvate thereof, (b) an SGLT-2 inhibitor, or a pharmaceutically acceptable salt or solvate thereof, and (c) a GLP-1 receptor agonist, or a pharmaceutically acceptable salt or solvate thereof, together are effective in treating NAFLD, the joint therapeutic effect of the combination of (a) the compound of Formula (I), or a pharmaceutically acceptable salt or solvate thereof, and (b) an SGLT-2 inhibitor, or a pharmaceutically acceptable salt or solvate thereof, and (c) a GLP-1 receptor agonist, or a pharmaceutically acceptable salt or solvate thereof, is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of the compound of Formula (I), or a pharmaceutically acceptable salt or solvate thereof alone.

In some embodiments, the amounts of (a) the compound of Formula (I), or a pharmaceutically acceptable salt or solvate thereof, (b) an SGLT-2 inhibitor, or a pharmaceutically acceptable salt or solvate thereof, (c) a GLP-1 receptor agonist, or a pharmaceutically acceptable salt or solvate thereof, and (d) metformin, or a pharmaceutically acceptable salt thereof, together are effective in treating NAFLD, the joint therapeutic effect of the combination of (a) the compound of Formula (I), or a pharmaceutically acceptable salt or solvate thereof, (b) an SGLT-2 inhibitor, or a pharmaceutically acceptable salt or solvate thereof, (c) a GLP-1 receptor agonist, or a pharmaceutically acceptable salt or solvate thereof, and (d) metformin, or a pharmaceutically acceptable salt thereof, is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of the compound of Formula (I), or a pharmaceutically acceptable salt or solvate thereof alone.

In some embodiments, wherein the amounts of (a) the compound of Formula (I), or a pharmaceutically acceptable salt or solvate thereof, and (b) an SGLT-2 inhibitor, or a pharmaceutically acceptable salt or solvate thereof, and/or (c) a GLP-1 receptor agonist, or the pharmaceutically acceptable salt or solvate thereof, and/or (d) metformin, or a pharmaceutically acceptable salt thereof, together are effective in treating NAFLD, the joint therapeutic effect of the combination of (a) the compound of Formula (I), or a pharmaceutically acceptable salt or solvate thereof, and (b) an SGLT-2 inhibitor, or a pharmaceutically acceptable salt or solvate thereof, and/or (c) a GLP-1 receptor agonist, or a pharmaceutically acceptable salt or solvate thereof, and/or (d) metformin, or a pharmaceutically acceptable salt thereof, is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of the SGLT-2 inhibitor alone, or the pharmaceutically acceptable salt or solvate thereof, or the GLP-1 receptor agonist alone, or the pharmaceutically acceptable salt or solvate thereof, or metformin alone, or the pharmaceutically acceptable salt thereof.

The term “preventing” as used herein means the prevention of the onset, recurrence or spread, in whole or in part, of the disease or condition as described herein, or a symptom thereof.

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, “subject” or “patient” refers to any subject, particularly a mammalian subject, for whom diagnosis, prognosis, or therapy is desired, for example, a human.

The terms “treatment regimen” and “dosing regimen” are used interchangeably to refer to the dose and timing of administration of each therapeutic agent in a combination of the invention.

The term “pharmaceutical combination”, as used herein, refers to a pharmaceutical treatment resulting from the mixing or combining of more than one active ingredient and includes both fixed and non-fixed combinations of the active ingredients.

The term “combination therapy” as used herein refers to a dosing regimen of two different therapeutically active agents (i.e., the components or combination partners of the combination) (e.g., the compound of Formula (I), or a pharmaceutically acceptable salt or solvate thereof, and an SGLT-2 inhibitor, a GLP-1 receptor agonist, and/or metformin, or both an SGLT-2 inhibitor and a GLP-1 receptor agonist, or each of an SGLT-2 inhibitor, a GLP-1 receptor agonist, and metformin), wherein the therapeutically active agents are administered together or separately in a manner prescribed by a medical care taker or according to a regulatory agency as defined herein. In one embodiment, a combination therapy comprises a combination of the compound of Formula (I), or a pharmaceutically acceptable salt or solvate thereof, and SGLT-2 inhibitor (e.g., empagliflozin). In one embodiment, a combination therapy consists essentially of a combination of (a) the compound of Formula (I), or a pharmaceutically acceptable salt or solvate thereof, and (b) an SGLT-2 inhibitor, or a pharmaceutically acceptable salt or solvate thereof (e.g., empagliflozin). In one embodiment, a combination therapy comprises a combination of (a) the compound of Formula (I), or a pharmaceutically acceptable salt or solvate thereof, and (b) a GLP-1 receptor agonist, or a pharmaceutically acceptable salt or solvate thereof, (e.g., liraglutide). In one embodiment, a combination therapy comprises a combination of (a) the compound of Formula (I), or a pharmaceutically acceptable salt or solvate thereof, (b) an SGLT-2 inhibitor, or a pharmaceutically acceptable salt or solvate thereof (e.g., empagliflozin), and (c) a GLP-1 receptor agonist, or a pharmaceutically acceptable salt or solvate thereof (e.g., liraglutide). In one embodiment, a combination therapy consists essentially of a combination of (a) the compound of Formula (I), or a pharmaceutically acceptable salt or solvate thereof, (b) an SGLT-2 inhibitor, or a pharmaceutically acceptable salt or solvate thereof (e.g., empagliflozin), and (c) a GLP-1 receptor agonist, or a pharmaceutically acceptable salt or solvate thereof (e.g., liraglutide). In one embodiment, a combination therapy comprises a combination of (a) the compound of Formula (I), or a pharmaceutically acceptable salt or solvate thereof, (b) an SGLT-2 inhibitor, or a pharmaceutically acceptable salt or solvate thereof (e.g., empagliflozin), (c) a GLP-1 receptor agonist, or a pharmaceutically acceptable salt or solvate thereof (e.g., liraglutide), and (d) metformin, or a pharmaceutically acceptable salt thereof. In one embodiment, a combination therapy consists essentially of a combination of (a) the compound of Formula (I), or a pharmaceutically acceptable salt or solvate thereof, (b) an SGLT-2 inhibitor, or a pharmaceutically acceptable salt or solvate thereof (e.g., empagliflozin), (c) a GLP-1 receptor agonist, or a pharmaceutically acceptable salt or solvate thereof (e.g., liraglutide), and (d) metformin, or a pharmaceutically acceptable salt thereof.

The term “fixed combination” means that the compound of Formula (I), or a pharmaceutically acceptable salt or solvate thereof, and at least one additional therapeutic agent (e.g., an SGLT-2 inhibitor, a GLP-1 receptor agonist, and/or metformin, both an SGLT-2 inhibitor and a GLP-1 receptor agonist, or each of an SGLT-2 inhibitor, a GLP-1 receptor agonist, and metformin), are both administered to a subject simultaneously in the form of a single composition or dosage.

The term “non-fixed combination” means that the compound of Formula (I), or a pharmaceutically acceptable salt or solvate thereof, and at least one additional therapeutic agent (e.g., an SGLT-2 inhibitor, a GLP-1 receptor agonist, both an SGLT-2 inhibitor and a GLP-1 receptor agonist, or each of an SGLT-2 inhibitor, a GLP-1 receptor agonist, and metformin) are formulated as separate compositions or dosages such that they may be administered to a subject in need thereof concurrently or sequentially with variable intervening time limits, wherein such administration provides effective levels of the two or more compounds in the body of the subject. These also apply to cocktail therapies, e.g. the administration of three or more active ingredients.

As can be appreciated in the art, a combination therapy can be administered to a patient for a period of time. In some embodiments, the period of time occurs following the administration of a different therapeutic treatment/agent or a different combination of therapeutic treatments/agents to the patient. In some embodiments, the period of time occurs before the administration of a different therapeutic treatment/agent or a different combination of therapeutic treatments/agents to the subject.

A suitable period of time can be determined by one skilled in the art (e.g., a physician). As can be appreciated in the art, a suitable period of time can be determined by one skilled in the art based on one or more of: the stage of disease in the patient, the mass and sex of the patient, clinical trial guidelines (e.g., those on the fda.gov website), and information on the approved drug label. For example a suitable period of time can be, e.g., from 1 week to 2 years, 1 week to 22 months, 1 week to 20 months, 1 week to 18 months, 1 week to 16 months, 1 week to 14 months, 1 week to 12 months, 1 week to 10 months, 1 week to 8 months, 1 week to 6 months, 1 week to 4 months 1 week to 2 months, 1 week to 1 month, 2 weeks to 2 years, 2 weeks to 22 months, 2 weeks to 20 months, 2 weeks to 18 months, 2 weeks to 16 months, 2 weeks to 14 months, 2 weeks to 12 months, 2 weeks to 10 months, 2 weeks to 8 months, 2 weeks to 6 months, 2 weeks to 4 months, 2 weeks to 2 months, 2 weeks to 1 month, 1 month to 2 years, 1 month to 22 months, 1 month to 20 months, 1 month to 18 months, 1 month to 16 months, 1 month to 14 months, 1 month to 12 months, 1 month to 10 months, 1 month to 8 months, 1 month to 6 months, 1 month to 4 months, 1 month to 2 months, 2 months to 2 years, 2 months to 22 months, 2 months to 20 months, 2 months to 18 months, 2 months to 16 months, 2 months to 14 months, 2 months to 12 months, 2 months to 10 months, 2 months to 8 months, 2 months to 6 months, 2 months to 4 months, 3 months to 2 years, 3 months to 22 months, 3 months to 20 months, 3 months to 18 months, 3 months to 16 months, 3 months to 14 months, 3 months to 12 months, 3 months to 10 months, 3 months to 8 months, 3 months to 6 months, 4 months to 2 years, 4 months to 22 months, 4 months to 20 months, 4 months to 18 months, 4 months to 16 months, 4 months to 14 months, 4 months to 12 months, 4 months to 10 months, 4 months to 8 months, 4 months to 6 months, 6 months to 2 years, 6 months to 22 months, 6 months to 20 months, 6 months to 18 months, 6 months to 16 months, 6 months to 14 months, 6 months to 12 months, 6 months to 10 months, 6 months to 8 months, 8 months to 2 years, 8 months to 22 months, 8 months to 20 months, 8 months to 18 months, 8 months to 16 months, 8 months to 14 months, 8 months to 12 months, 8 months to 10 months, 10 months to 2 years, 10 months to 22 months, 10 months to 20 months, 10 months to 18 months, 10 months to 16 months, 10 months to 14 months, 10 months to 12 months, 12 months to 2 years, 12 months to 22 months, 12 months to 20 months, 12 months to 18 months, 12 months to 16 months, or 12 months to 14 months, inclusive. In some embodiments, a suitable period of time can be, e.g., from 1 month to 10 years, 1 month to 5 years, 5 years to 10 years, 3 years to 7 years, 1 year to 3 years, 3 years to 6 years, 6 years to 9 years, 2 years to 3 years, 3 years to 4 years, 4 years to 5 years, 5 years to 6 years, 6 years to 7 years, 7 years to 8 years, 8 years to 9 years, or 9 years to 10 years.

The phrases “prior to a period of time” or “before a period of time” refer to (1) the completion of administration of treatment to the subject before the first administration of a therapeutic agent during the period of time, and/or (2) the administration of one or more therapeutic agents to the subject before a first administration of a therapeutic agent in the combination therapy described herein during the period of time, such that the one or more therapeutic agents are present in subtherapeutic and/or undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic and/or undetectable levels in the subject at the time the first administration of a therapeutic agent in the combination therapy is performed during the period of time.

The term “synergy” or “synergistic” is used herein to mean that the effect of the combination of the two therapeutic agents of the combination therapy is greater than the sum of the effect of each agent when administered alone. A “synergistic amount” or “synergistically effective amount” is an amount of the combination of the two combination partners that results in a synergistic effect, as “synergistic” is defined herein. Determining a synergistic interaction between two combination partners, the optimum range for the effect and absolute dose ranges of each component for the effect may be definitively measured by administration of the combination partners over different w/w (weight per weight) ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods. Exemplary synergistic effects includes, but are not limited to, enhanced therapeutic efficacy, decreased dosage at equal or increased level of efficacy, reduced or delayed development of drug resistance, and simultaneous enhancement or equal therapeutic actions (e.g., the same therapeutic effect as at least one of the therapeutic agents) and reduction of unwanted drug effects (e.g. side effects and adverse events) of at least one of the therapeutic agents.

For example, a synergistic ratio of two therapeutic agents can be identified by determining a synergistic effect in, for example, an art-accepted in vivo model (e.g., an animal model) of NAFLD (e.g., the diet induced obese (DIO)-NASH mouse model or any of the models described in Herck et al. Nutrients. 2017 October; 9(10): 1072, which is incorporated by reference herein in its entirety).

In some embodiments, “synergistic effect” as used herein refers to a combination of (a) the compound of Formula (I), or a pharmaceutically acceptable salt or solvate thereof, and (b) an SGLT-2 inhibitor, and/or (c) a GLP-1 receptor agonist, and/or (d) metformin, producing an effect, for example, any of the beneficial or desired results including clinical results as described herein, for example slowing the symptomatic progression of NAFLD, or symptoms thereof, which is greater than the sum of effect observed when the compound of Formula (I), or a pharmaceutically acceptable salt or solvate thereof, and the SGLT-2 inhibitor, and/or the GLP-1 receptor agonist, and/or metformin, are administered alone. In some embodiments, “synergistic effect” as used herein refers to a combination of (a) the compound of Formula (I), or a pharmaceutically acceptable salt or solvate thereof, and (b) an SGLT-2 inhibitor, producing an effect, for example, any of the beneficial or desired results including clinical results as described herein, for example slowing the symptomatic progression of NAFLD, or symptoms thereof, which is greater than the sum of effect observed when the compound of Formula (I), or a pharmaceutically acceptable salt or solvate thereof, and the SGLT-2 inhibitor are administered alone.

In other embodiments, “synergistic effect” as used herein refers to a combination of (a) the compound of Formula (I), or a pharmaceutically acceptable salt or solvate thereof, and (c) a GLP-1 receptor agonist, producing an effect, for example, any of the beneficial or desired results including clinical results as described herein, for example slowing the symptomatic progression of NAFLD, or symptoms thereof, which is greater than the sum of effect observed when the compound of Formula (I), or a pharmaceutically acceptable salt or solvate thereof, and the GLP-1 receptor agonist are administered alone.

In still other embodiments, “synergistic effect” as used herein refers to a combination of (a) the compound of Formula (I), or a pharmaceutically acceptable salt or solvate thereof, (b) an SGLT-2 inhibitor, and (c) a GLP-1 receptor agonist producing an effect, for example, any of the beneficial or desired results including clinical results as described herein, for example slowing the symptomatic progression of NAFLD, or symptoms thereof, which is greater than the sum of effect observed when the compound of Formula (I), or a pharmaceutically acceptable salt or solvate thereof, the SGLT-2 inhibitor or the GLP-1 receptor agonist are administered alone.

In some embodiments, “synergistic effect” as used herein refers to a combination of (a) the compound of Formula (I), or a pharmaceutically acceptable salt or solvate thereof, (b) an SGLT-2 inhibitor, (c) a GLP-1 receptor agonist, and (d) metformin, or a pharmaceutically acceptable salt thereof, producing an effect, for example, any of the beneficial or desired results including clinical results as described herein, for example slowing the symptomatic progression of NAFLD, or symptoms thereof, which is greater than the sum of effect observed when the compound of Formula (I), or a pharmaceutically acceptable salt or solvate thereof, the SGLT-2 inhibitor, the GLP-1 receptor agonist, and the metformin, are administered alone.

In some embodiments, “synergistic effect” as used herein refers to a combination of (a) the compound of Formula (I), or a pharmaceutically acceptable salt or solvate thereof, (b) an SGLT-2 inhibitor, and (d) metformin, or a pharmaceutically acceptable salt thereof, producing an effect, for example, any of the beneficial or desired results including clinical results as described herein, for example slowing the symptomatic progression of NAFLD, or symptoms thereof, which is greater than the sum of effect observed when the compound of Formula (I), or a pharmaceutically acceptable salt or solvate thereof, the SGLT-2 inhibitor, and the metformin, are administered alone.

In some embodiments, “synergistic effect” as used herein refers to a combination of (a) the compound of Formula (I), or a pharmaceutically acceptable salt or solvate thereof, (c) a GLP-1 receptor agonist, and (d) metformin, or a pharmaceutically acceptable salt thereof, producing an effect, for example, any of the beneficial or desired results including clinical results as described herein, for example slowing the symptomatic progression of NAFLD, or symptoms thereof, which is greater than the sum of effect observed when the compound of Formula (I), or a pharmaceutically acceptable salt or solvate thereof, the GLP-1 receptor agonist, and the metformin, are administered alone.

In any of the embodiments described herein, various combinations of the compound of Formula (I), or a pharmaceutically acceptable salt or solvate thereof, an SGLT-2 inhibitor, a GLP-1 receptor agonist, and metformin, or a pharmaceutically acceptable salt thereof, producing an effect, are contemplated. For example, the compound of formula (I) and an SGLT-2 inhibitor; the compound of formula (I) and a GLP-1 receptor agonist; the compound of formula (I), an SGLT-2 inhibitor, and a GLP-1 receptor agonist; the compound of formula (I), an SGLT-2 inhibitor, a GLP-1 receptor agonist, and metformin; the compound of formula (I), an SGLT-2 inhibitor, and metformin; and the compound of formula (I), a GLP-1 receptor agonist, and metformin; wherein these combinations produce an effect, for example, any of the beneficial or desired results including clinical results as described herein, for example slowing the symptomatic progression of NAFLD, or symptoms thereof, which is greater than the sum of the effect observed when the same amount of the compound of formula (I) as in the combination, or a pharmaceutically acceptable salt or solvate thereof, and the same amount of the SGLT-2 inhibitor, GLP-1 receptor agonist, and/or metformin as in the combination are administered alone. These combinations produce an effect, for example, a therapeutic effect using a smaller dose of either, or both, or all of the specific compounds as a monotherapy. For example, producing a therapeutic effect using a smaller dose of (a) the compound of Formula (I), or a pharmaceutically acceptable salt or solvate thereof, and (b) the SGLT-2 inhibitor compared to the amount used in monotherapy; (a) the compound of Formula (I), or a pharmaceutically acceptable salt or solvate thereof, and (c) the GLP-1 receptor agonist; (a) the compound of Formula (I), or a pharmaceutically acceptable salt or solvate thereof, (b) the SGLT-2 inhibitor, and (c) the GLP-1 receptor agonist; (a) the compound of Formula (I), or a pharmaceutically acceptable salt or solvate thereof, (b) the SGLT-2 inhibitor, and (c) the GLP-1 receptor agonist, and (d) metformin; compared to the amount of each used in monotherapy; (a) the compound of Formula (I), or a pharmaceutically acceptable salt or solvate thereof, (b) the SGLT-2 inhibitor, and (d) metformin; (a) the compound of Formula (I), or a pharmaceutically acceptable salt or solvate thereof, (c) the GLP-1 receptor agonist, and (d) metformin; compared to the amount of each used in monotherapy. For example, the dose of the compound of Formula (I), or a pharmaceutically acceptable salt or solvate thereof, administered in combination with an SGLT-2 inhibitor and/or a GLP-1 receptor agonist and/or metformin may be about 0.5% to about 90% of the dose of the compound of Formula (I) administered as a monotherapy to produce the same therapeutic effect, e.g., any of the beneficial or desired results including clinical results as described herein, for example slowing the symptomatic progression of NAFLD, or symptoms thereof.

For example, the dose of the compound of Formula (I) administered in combination with an SGLT-2 inhibitor and/or a GLP-1 receptor agonist and/or metformin may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of the compound of Formula (I) administered as a monotherapy. As another example, the dose of the SGLT-2 inhibitor and/or GLP-1 receptor agonist and/or metformin administered in combination with the compound of Formula (I), or a pharmaceutically acceptable salt or solvate thereof, may be about 0.5% to about 90% of the dose of the SGLT-2 inhibitor or GLP-1 receptor agonist administered as a monotherapy to produce the same therapeutic effect, e.g., any of the beneficial or desired results including clinical results as described herein, for example slowing the symptomatic progression of NAFLD, or symptoms thereof. For example, the dose of the SGLT-2 and/or GLP-1 receptor agonist and/or metformin administered in combination with the compound of Formula (I) may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of the SGLT-2 inhibitor and/or GLP-1 receptor agonist and/or metformin administered as a monotherapy.

In some embodiments, “synergistic effect” as used herein refers to a combination of (a) the compound of Formula (I), or a pharmaceutically acceptable salt or solvate thereof, (b) an SGLT-2 inhibitor, and (c) a GLP-1 receptor agonist; or a combination of (a) the compound of Formula (I), or a pharmaceutically acceptable salt or solvate thereof and (b) an SGLT-2 inhibitor; or a combination of (a) the compound of Formula (I), or a pharmaceutically acceptable salt or solvate thereof and (c) a GLP-1 receptor agonist; or a combination of (a) the compound of Formula (I), or a pharmaceutically acceptable salt or solvate thereof, (b) an SGLT-2 inhibitor, (c) a GLP-1 receptor agonist, and (d) metformin; or a combination of (a) the compound of Formula (I), or a pharmaceutically acceptable salt or solvate thereof, (b) an SGLT-2 inhibitor, and (d) metformin; or a combination of (a) the compound of Formula (I), or a pharmaceutically acceptable salt or solvate thereof, (c) a GLP-1 receptor agonist, and (d) metformin; producing, for example, a therapeutic effect using a smaller dose of one or more of (a) the compound of Formula (I), or a pharmaceutically acceptable salt or solvate thereof, (b) the SGLT-2 inhibitor, and/or (c) GLP-1 receptor agonist and/or (d) metformin, compared to the amount used in monotherapy. For example, the dose of the compound of Formula (I), or a pharmaceutically acceptable salt or solvate thereof, administered in combination with an SGLT-2 inhibitor and/or a GLP-1 receptor agonist and/or metformin, may be about 0.5% to about 90% of the dose of the compound of Formula (I) administered as a monotherapy to produce the same therapeutic effect, e.g., any of the beneficial or desired results including clinical results as described herein, for example slowing the symptomatic progression of NAFLD, or symptoms thereof. For example, the dose of the compound of Formula (I) administered in combination with an SGLT-2 inhibitor and/or a GLP-1 receptor agonist and/or metformin, may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of the compound of Formula (I) administered as a monotherapy. Similarly, the dose of the SGLT-2 inhibitor administered in combination with the compound of Formula (I), or a pharmaceutically acceptable salt or solvate thereof, a GLP-1 receptor agonist and/or metformin, may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of the SGLT-2 inhibitor administered as a monotherapy. Likewise, the dose of the GLP-1 receptor agonist administered in combination with the compound of Formula (I), or a pharmaceutically acceptable salt or solvate thereof, an SGLT-2 inhibitor and/or metformin, may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of the GLP-1 receptor agonist administered as a monotherapy. The dose of metformin administered in combination with the compound of Formula (I), or a pharmaceutically acceptable salt or solvate thereof, an SGLT-2 inhibitor and/or a GLP-1 receptor agonist, may also be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of the metformin administered as a monotherapy.

In some more particular embodiments, “synergistic effect” as used herein refers to a combination of (a) the compound of Formula (I), or a pharmaceutically acceptable salt or solvate thereof, and (b) an SGLT-2 inhibitor or a GLP-1 receptor agonist producing a desired therapeutic effect and a reduction in an unwanted drug effect, side effect, or adverse event. In other more particular embodiments, “synergistic effect” as used herein refers to a combination of (a) the compound of Formula (I), or a pharmaceutically acceptable salt or solvate thereof, and (b) an SGLT-2 inhibitor, producing a desired therapeutic effect and a reduction in an unwanted drug effect, side effect, or adverse event. In still other more particular embodiments, “synergistic effect” as used herein refers to a combination of (a) the compound of Formula (I), or a pharmaceutically acceptable salt or solvate thereof, (b) an SGLT-2 inhibitor, and (c) a GLP-1 receptor agonist producing a desired therapeutic effect and a reduction in an unwanted drug effect, side effect, or adverse event. In some more particular embodiments, “synergistic effect” as used herein refers to a combination of (a) the compound of Formula (I), or a pharmaceutically acceptable salt or solvate thereof, (b) an SGLT-2 inhibitor or a GLP-1 receptor agonist, and (d) metformin, producing a desired therapeutic effect and a reduction in an unwanted drug effect, side effect, or adverse event. In other more particular embodiments, “synergistic effect” as used herein refers to a combination of (a) the compound of Formula (I), or a pharmaceutically acceptable salt or solvate thereof, (b) an SGLT-2 inhibitor, (c) a GLP-1 receptor agonist, and (d) metformin, producing a desired therapeutic effect and a reduction in an unwanted drug effect, side effect, or adverse event. In still other more particular embodiments, “synergistic effect” as used herein refers to a combination of (a) the compound of Formula (I), or a pharmaceutically acceptable salt or solvate thereof and (c) a GLP-1 receptor agonist, producing a desired therapeutic effect and a reduction in an unwanted drug effect, side effect, or adverse event.

In some embodiments, the desired therapeutic effect is the same therapeutic effect observed in monotherapy of the compound of Formula (I), or a pharmaceutically acceptable salt or solvate thereof, an SGLT-2 inhibitor, or a GLP-1 receptor agonist, e.g., any of the beneficial or desired results including clinical results as described herein, for example slowing the symptomatic progression of NAFLD, or symptoms thereof. In other embodiments, the desired therapeutic effect is the same therapeutic effect observed in monotherapy of the compound of Formula (I), or a pharmaceutically acceptable salt or solvate thereof, an SGLT-2 inhibitor or a GLP-1 receptor agonist, and/or metformin, e.g., any of the beneficial or desired results including clinical results as described herein, for example slowing the symptomatic progression of NAFLD, or symptoms thereof.

In some embodiments, an unwanted drug effect, side effect, or adverse event is associated with or observed in monotherapy of the compound of Formula (I), or a pharmaceutically acceptable salt or solvate thereof, an SGLT-2 inhibitor, or a GLP-1 receptor agonist. In some embodiments, an unwanted drug effect, side effect, or adverse event is associated with or observed in monotherapy of the compound of Formula (I), or a pharmaceutically acceptable salt or solvate thereof includes, but is not limited to edema, weight gain, hypertension, cardiovascular disease, and cardiovascular events (e.g. cardiovascular death, nonfatal myocardial infarction and nonfatal stroke). In some embodiments, an unwanted drug effect, side effect, or adverse event is associated with or observed in monotherapy of the compound of Formula (I), or a pharmaceutically acceptable salt or solvate thereof, an SGLT-2 inhibitor and/or a GLP-1 receptor agonist, and/or metformin. In some embodiments, an unwanted drug effect, side effect, or adverse event is associated with or observed in monotherapy of the compound of Formula (I), or a pharmaceutically acceptable salt or solvate thereof includes, but is not limited to edema, weight gain, hypertension, cardiovascular disease, and cardiovascular events (e.g. cardiovascular death, nonfatal myocardial infarction and nonfatal stroke).

### Methods and Combination Therapies

The present disclosure relates to methods and combination therapies for treating non-alcoholic fatty liver disease (NAFLD) in a subject in need thereof by administering (a) the compound of Formula (I):

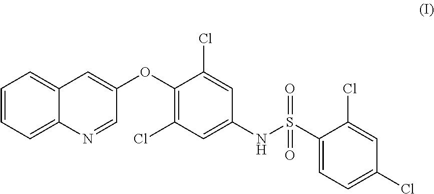 [SMILES: O=S(=O)(Nc1cc(Cl)c(Oc2cnc3ccccc3c2)c(Cl)c1)c1ccc(Cl)cc1Cl]

or a pharmaceutically acceptable salt or solvate thereof, (b) a sodium-glucose cotransporter (SGLT) inhibitor, or a pharmaceutically acceptable salt or solvate thereof, and/or a glucagon-like peptide-1 (GLP-1) agonist, or a pharmaceutically acceptable salt or solvate thereof, and optionally (d) metformin, or a pharmaceutically acceptable salt thereof. In some embodiments, the SGLT inhibitor is a sodium-glucose cotransporter-2 (SGLT-2) inhibitor.

In some embodiments, the present disclosure relates to methods and combination therapies for treating non-alcoholic fatty liver disease (NAFLD) in a subject in need thereof by administering (a) the compound of Formula (I), or a pharmaceutically acceptable salt or solvate thereof, (b) an SGLT inhibitor, or a pharmaceutically acceptable salt or solvate thereof, and (c) a GLP-1 receptor agonist, or a pharmaceutically acceptable salt or solvate thereof. In other embodiments, the present disclosure relates to methods and combination therapies for treating non-alcoholic fatty liver disease (NAFLD) in a subject in need thereof by administering (a) the compound of Formula (I), or a pharmaceutically acceptable salt or solvate thereof, (b) an SGLT inhibitor, or a pharmaceutically acceptable salt or solvate thereof, (c) a GLP-1 receptor agonist, or a pharmaceutically acceptable salt or solvate thereof, and (d) metformin, or a pharmaceutically acceptable salt thereof. In still other embodiments, the present disclosure relates to methods and combination therapies for treating non-alcoholic fatty liver disease (NAFLD) in a subject in need thereof by administering (a) the compound of Formula (I), or a pharmaceutically acceptable salt or solvate thereof and (b) an SGLT inhibitor, or a pharmaceutically acceptable salt or solvate thereof. In some embodiments, the present disclosure relates to methods and combination therapies for treating non-alcoholic fatty liver disease (NAFLD) in a subject in need thereof by administering (a) the compound of Formula (I), or a pharmaceutically acceptable salt or solvate thereof and (c) a GLP-1 receptor agonist, or a pharmaceutically acceptable salt or solvate thereof. In other embodiments, the present disclosure relates to methods and combination therapies for treating non-alcoholic fatty liver disease (NAFLD) in a subject in need thereof by administering (a) the compound of Formula (I), or a pharmaceutically acceptable salt or solvate thereof, (b) an SGLT inhibitor, or a pharmaceutically acceptable salt or solvate thereof, and (d) metformin, or a pharmaceutically acceptable salt thereof. In still other embodiments, the present disclosure relates to methods and combination therapies for treating non-alcoholic fatty liver disease (NAFLD) in a subject in need thereof by administering (a) the compound of Formula (I), or a pharmaceutically acceptable salt or solvate thereof, (c) a GLP-1 receptor agonist, or a pharmaceutically acceptable salt or solvate thereof, and (d) metformin, or a pharmaceutically acceptable salt thereof. In some embodiments, the SGLT inhibitor is a SGLT-2 inhibitor such as dapagliflozin (e.g., dapagliflozin propylene glycol hydrate) or empagliflozin. In some embodiments, the GLP-1 receptor agonist is liraglutide.

NAFLD is characterized by hepatic steatosis with no secondary causes of hepatic steatosis including excessive alcohol consumption, other known liver diseases, or long-term use of a steatogenic medication (Chalasani et al., Hepatology. 2018, 67(1):328-357, which is hereby incorporated by reference in its entirety). NAFLD can be categorized into non-alcoholic fatty liver (NAFL) and non-alcoholic steatohepatitis (NASH). According to Chalasani et al., NAFL is defined as the presence of ≥5% hepatic steatosis without evidence of hepatocellular injury in the form of hepatocyte ballooning. NASH is defined as the presence of ≥5% hepatic steatosis and inflammation with hepatocyte injury (e.g., ballooning), with or without any liver fibrosis. Additionally, NASH is commonly associated with hepatic inflammation and liver fibrosis, which can progress to cirrhosis, end-stage liver disease, and hepatocellular carcinoma. However, liver fibrosis is not always present in NASH, but the severity of fibrosis can be linked to long-term outcomes.

There are many approaches used to assess and evaluate whether a subject has NAFLD and if so, the severity of the disease including differentiating whether the NAFLD is NAFL or NASH.

For example, these approaches include determining one or more of hepatic steatosis (e.g., accumulation of fat in the liver); the NAFLD Activity Score (NAS); hepatic inflammation; biomarkers indicative of one or more of liver damage, hepatic inflammation, liver fibrosis, and/or liver cirrhosis (e.g., serum markers and panels); and liver fibrosis and/or cirrhosis. Further examples of physiological indicators of NAFLD can include liver morphology, liver stiffness, and the size or weight of the subject's liver. In some embodiments, NAFLD in the subject is evidenced by an accumulation of hepatic fat and detection of a biomarker indicative of liver damage. For example, elevated serum ferritin and low titers of serum autoantibodies can be common features of NAFLD. In some embodiments, methods to assess NAFLD include magnetic resonance imaging, either by spectroscopy or by proton density fat fraction (MRI-PDFF) to quantify steatosis, transient elastography (FIBROSCAN®), hepatic venous pressure gradient (HPVG), hepatic stiffness measurement with MRE for diagnosing significant liver fibrosis and/or cirrhosis, and assessing histological features of liver biopsy. In some embodiments, magnetic resonance imaging is used to detect one or more of steatohepatitis (NASH-MRI), liver fibrosis (Fibro-MRI), and steatosis see, for example, U.S. Application Publication Nos. 2016/146715 and 2005/0215882, each of which are incorporated herein by reference in their entireties. In some embodiments, treatment of NAFLD comprises one or more of a decrease in symptoms; a reduction in the amount of hepatic steatosis; a decrease in the NAS; a decrease in hepatic inflammation; a decrease in the level of biomarkers indicative of one or more of liver damage, inflammation, liver fibrosis, and/or liver cirrhosis; and a reduction in fibrosis and/or cirrhosis, a lack of further progression of fibrosis and/or cirrhosis, or a slowing of the progression of fibrosis and/or cirrhosis.

In some embodiments, treatment of NAFLD comprises a decrease of one or more symptoms associated with NAFLD in the subject. Exemplary symptoms can include one or more of an enlarged liver, fatigue, pain in the upper right abdomen, abdominal swelling, enlarged blood vessels just beneath the skin's surface, enlarged breasts in men, enlarged spleen, red palms, jaundice, and pruritus. In some embodiments, the subject is asymptomatic. In some embodiments, the total body weight of the subject does not increase. In some embodiments, the total body weight of the subject decreases. In some embodiments, the body mass index (BMI) of the subject does not increase. In some embodiments, the body mass index (BMI) of the subject decreases. In some embodiments, the waist and hip (WTH) ratio of the subject does not increase. In some embodiments, the waist and hip (WTH) ratio of the subject decreases.

In some embodiments, hepatic steatosis is determined by one or more methods selected from the group consisting of ultrasonography, computed tomography (CT), magnetic resonance imaging, magnetic resonance spectroscopy (MRS), magnetic resonance elastography (MRE), transient elastography (TE) (e.g., FIBROSCAN®), measurement of liver size or weight, or by liver biopsy (see, e.g., Di Lascio et al., Ultrasound Med Biol. 2018 August; 44(8):1585-1596; Lv et al., J Clin Transl Hepatol. 2018 Jun. 28; 6(2): 217-221; Reeder, et al., J Magn Reson Imaging. 2011 October; 34(4): spcone; and de Ledinghen V, et al., J Gastroenterol Hepatol. 2016 April; 31(4):848-55, each of which are incorporated herein by reference in their entireties). A subject diagnosed with NAFLD can have more than about 5% hepatic steatosis, for example, about 5% to about 25%, about 25% to about 45%, about 45% to about 65%, or greater than about 65% hepatic steatosis. In some embodiments, a subject with about 5% to about 33% hepatic steatosis has stage 1 hepatic steatosis, a subject with about 33% to about 66% hepatic steatosis has stage 2 hepatic steatosis, and a subject with greater than about 66% hepatic steatosis has stage 3 hepatic steatosis. In some embodiments, treatment of NAFLD can be assessed by measuring hepatic steatosis. In some embodiments, treatment of NAFLD comprises a reduction in hepatic steatosis following administration of one or more compounds described herein.

In some embodiments, the amount of hepatic steatosis is determined prior to administration of the combination of (a) the compound of Formula (I), or a pharmaceutically acceptable salt or solvate thereof, and (b) an SGLT-2 inhibitor or a GLP-1 receptor agonist. In some embodiments, the amount of hepatic steatosis is determined prior to administration of the combination of (a) the compound of Formula (I), or a pharmaceutically acceptable salt or solvate thereof, (b) an SGLT-2 inhibitor, and (c) a GLP-1 receptor agonist. In other embodiments, the amount of hepatic steatosis is determined prior to administration of the combination of (a) the compound of Formula (I), or a pharmaceutically acceptable salt or solvate thereof and (b) an SGLT-2 inhibitor. In still other embodiments, the amount of hepatic steatosis is determined prior to administration of the combination of (a) the compound of Formula (I), or a pharmaceutically acceptable salt or solvate thereof and (c) a GLP-1 receptor agonist. In some embodiments, the amount of hepatic steatosis is determined prior to administration of the combination of (a) the compound of Formula (I), or a pharmaceutically acceptable salt or solvate thereof, (b) an SGLT-2 inhibitor, (c) a GLP-1 receptor agonist, and (d) metformin. In other embodiments, the amount of hepatic steatosis is determined prior to administration of the combination of (a) the compound of Formula (I), or a pharmaceutically acceptable salt or solvate thereof, (b) an SGLT-2 inhibitor, and (d) metformin. In still other embodiments, the amount of hepatic steatosis is determined prior to administration of the combination of (a) the compound of Formula (I), or a pharmaceutically acceptable salt or solvate thereof, (c) a GLP-1 receptor agonist, and (d) metformin. In some embodiments, the amount of hepatic steatosis is determined during the period of time or after the period of time of administration of the combination of (a) and (b), the combination of (a), (b), and (c), the combination of (a) and (c), the combination of (a), (b), and (d), the combination of (a), (c), and (d), or the combination of (a), (b), (c), and (d). In some embodiments, a reduction in the amount of hepatic steatosis during the period of time or after the period of time of administration of the combination of (a) and (b), the combination of (a), (b), and (c), the combination of (a) and (c), the combination of (a), (b), and (d), the combination of (a), (c), and (d), or the combination of (a), (b), (c), and (d), compared to prior to administration of the combination of (a) and (b), the combination of (a), (b), and (c), the combination of (a) and (c), the combination of (a), (b), and (d), the combination of (a), (c), and (d), or the combination of (a), (b), (c), and (d), indicates treatment of NAFLD. For example, a reduction in the amount of hepatic steatosis by about 1% to about 50%, about 25% to about 75%, or about 50% to about 100% indicates treatment of NAFLD. In some embodiments, a reduction in the amount of hepatic steatosis by about 5%, bout 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of NAFLD.

In some embodiments, the severity of NALFD can be assessed using the NAS. In some embodiments, treatment of NAFLD can be assessed using the NAS. In some embodiments, treatment of NAFLD comprises a reduction in the NAS following administration of one or more compounds described herein. In some embodiments, the NAS can be determined as described in Kleiner et al., Hepatology. 2005, 41(6):1313-1321, which is hereby incorporated by reference in its entirety. See, for example, Table 2 for a simplified NAS scheme adapted from Kleiner.

TABLE 2
Example of the NAFLD Activity Score (NAS) with Fibrosis Stage

| Feature | Degree | Score |
|---|---|---|
| Steatosis | <5% | 0 |
| | 5-33% | 1 |
| | >33-66% | 2 |
| | >66% | 3 |
| Lobular | No foci | 0 |
| Inflammation | <2 foci/200x | 1 |
| | 2-4 foci/200x | 2 |
| | >4 foci/200x | 3 |
| Ballooning | None | 0 |
| degeneration | Few | 1 |
| | Many cells/Prominent ballooning | 2 |
| Fibrosis | None | 0 |
| | Perisinusoidal or periportal | 1 |
| | Perisinusoidal & portal/periportal | 2 |
| | Bridging fibrosis | 3 |
| | Cirrhosis | 4 |

In some embodiments, the NAS is determined non-invasively, for example, as described in U.S. Application Publication No. 2018/0140219, which is incorporated by reference herein in its entirety. In some embodiments, the NAS is determined for a sample from the subject prior to administration of the combination of (a) the compound of Formula (I), or a pharmaceutically acceptable salt or solvate thereof, (b) an SGLT-2 inhibitor or a GLP-1 receptor agonist, and/or (d) metformin. In some embodiments, a NAS is determined for a sample from the subject prior to administration of the combination of (a) the compound of Formula (I), or a pharmaceutically acceptable salt or solvate thereof, (b) an SGLT-2 inhibitor, and (c) a GLP-1 receptor agonist. In other embodiments, a NAS is determined for a sample from the subject prior to administration of the combination of (a) the compound of Formula (I), or a pharmaceutically acceptable salt or solvate thereof, (b) an SGLT-2 inhibitor, (c) a GLP-1 receptor agonist, and (d) metformin. In still other embodiments, a NAS is determined for a sample from the subject prior to administration of the combination of (a) the compound of Formula (I), or a pharmaceutically acceptable salt or solvate thereof and (b) an SGLT-2 inhibitor. In some embodiments, a NAS is determined for a sample from the subject prior to administration of the combination of (a) the compound of Formula (I), or a pharmaceutically acceptable salt or solvate thereof and (c) a GLP-1 receptor agonist. In other embodiments, a NAS is determined for a sample from the subject prior to administration of the combination of (a) the compound of Formula (I), or a pharmaceutically acceptable salt or solvate thereof, (b) an SGLT-2 inhibitor, and (d) metformin. In still other embodiments, a NAS is determined for a sample from the subject prior to administration of the combination of (a) the compound of Formula (I), or a pharmaceutically acceptable salt or solvate thereof, (c) a GLP-1 receptor agonist, and (d) metformin. In some embodiments, the NAS is determined during the period of time or after the period of time of administration of the combination of (a) and (b), the combination of (a), (b), and (c), the combination of (a) and (c), the combination of (a), (b), and (d), the combination of (a), (c), and (d), or the combination of (a), (b), (c), and (d). In some embodiments, a lower NAS score during the period of time or after the period of time of administration of the of the combination of (a) and (b), the combination of (a), (b), and (c), the combination of (a) and (c), the combination of (a), (b), and (d), the combination of (a), (c), and (d), or the combination of (a), (b), (c), and (d) compared to prior to administration of the of the combination of (a) and (b), the combination of (a), (b), and (c), the combination of (a) and (c), the combination of (a), (b), and (d), the combination of (a), (c), and (d), or the combination of (a), (b), (c), and (d) indicates treatment of NAFLD. For example, a decrease in the NAS by 1, by 2, by 3, by 4, by 5, by 6, or by 7 indicates treatment of NAFLD. In some embodiments, the NAS following administration of the of the combination of (a) and (b), the combination of (a), (b), and (c), or the combination of (a), (b), (c), the combination of (a) and (c), the combination of (a), (b), and (d), the combination of (a), (c), and (d), or the combination of (a), (b), (c), and (d) is 7 or less. In some embodiments, the NAS during the period of time of administration of the of the combination of (a) and (b), the combination of (a), (b), and (c), the combination of (a) and (c), the combination of (a), (b), and (d), the combination of (a), (c), and (d), or the combination of (a), (b), (c), and (d) is 5 or less, 4 or less, 3 or less, or 2 or less. In some embodiments, the NAS during the period of time of administration of the of the combination of (a) and (b), the combination of (a), (b), and (c), the combination of (a) and (c), the combination of (a), (b), and (d), the combination of (a), (c), and (d), or the combination of (a), (b), (c), and (d) is 7 or less. In some embodiments, the NAS during the period of time of administration of the of the combination of (a) and (b), the combination of (a), (b), and (c), or the combination of (a), (b), (c), the combination of (a) and (c), the combination of (a), (b), and (d), the combination of (a), (c), and (d), or the combination of (a), (b), (c), and (d) is 5 or less, 4 or less, 3 or less, or 2 or less. In some embodiments, the NAS after the period of time of administration of the of the combination of (a) and (b), the combination of (a), (b), and (c), the combination of (a) and (c), the combination of (a), (b), and (d), the combination of (a), (c), and (d), or the combination of (a), (b), (c), and (d) is 7 or less. In some embodiments, the NAS after the period of time of administration of the combination of (a) and (b), the combination of (a), (b), and (c), the combination of (a) and (c), the combination of (a), (b), and (d), the combination of (a), (c), and (d), or the combination of (a), (b), (c), and (d) is 5 or less, 4 or less, 3 or less, or 2 or less.

In some embodiments, the presence of hepatic inflammation is determined by one or more methods selected from the group consisting of biomarkers indicative of hepatic inflammation and a liver biopsy sample(s) from the subject. In some embodiments, the severity of hepatic inflammation is determined from a liver biopsy sample(s) from the subject. For example, hepatic inflammation in a liver biopsy sample can be assessed as described in Kleiner et al., Hepatology. 2005, 41(6):1313-1321 and Brunt et al., Am J Gastroenterol 1999, 94:2467-2474, each of which are hereby incorporated by reference in their entireties. In some embodiments, the severity of hepatic inflammation is determined prior to administration of the combination of (a) the compound of Formula (I), or a pharmaceutically acceptable salt or solvate thereof, and (b) an SGLT-2 inhibitor or a GLP-1 receptor agonist. In some embodiments, the severity of hepatic inflammation is determined prior to administration of the combination of (a) the compound of Formula (I), or a pharmaceutically acceptable salt or solvate thereof, (b) an SGLT-2 inhibitor, and (c) a GLP-1 receptor agonist. In some embodiments, the severity of hepatic inflammation is determined prior to administration of the combination of (a) the compound of Formula (I), or a pharmaceutically acceptable salt or solvate thereof, (b) an SGLT-2 inhibitor, (c) a GLP-1 receptor agonist, and (d) metformin. In other embodiments, the severity of hepatic inflammation is determined prior to administration of the combination of (a) the compound of Formula (I), or a pharmaceutically acceptable salt or solvate thereof and (b) an SGLT-2 inhibitor. In still other embodiments, the severity of hepatic inflammation is determined prior to administration of the combination of (a) the compound of Formula (I), or a pharmaceutically acceptable salt or solvate thereof and (c) a GLP-1 receptor agonist. In some embodiments, the severity of hepatic inflammation is determined prior to administration of the combination of (a) the compound of Formula (I), or a pharmaceutically acceptable salt or solvate thereof, (b) an SGLT-2 inhibitor, and (d) metformin. In other embodiments, the severity of hepatic inflammation is determined prior to administration of the combination of (a) the compound of Formula (I), or a pharmaceutically acceptable salt or solvate thereof, (c) a GLP-1 receptor agonist, and (d) metformin. In some embodiments, the severity of hepatic inflammation is determined during the period of time or after the period of time of administration of the combination of (a) and (b), the combination of (a), (b), and (c), the combination of (a) and (c), the combination of (a), (b), and (d), the combination of (a), (c), and (d), or the combination of (a), (b), (c), and (d). In some embodiments, a decrease in the severity of hepatic inflammation during the period of time or after the period of time of administration of the combination of (a) and (b), the combination of (a), (b), and (c), the combination of (a) and (c), the combination of (a), (b), and (d), the combination of (a), (c), and (d), or the combination of (a), (b), (c), and (d), compared to prior to administration of the combination of (a) and (b) or the combination of (a), (b), and (c) indicates treatment of NAFLD. For example, a decrease in the severity of hepatic inflammation by about 1% to about 50%, about 25% to about 75%, or about 50% to about 100% indicates treatment of NAFLD. In some embodiments, a decrease in the severity of hepatic inflammation by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of NAFLD.

In some embodiments, treatment of NAFLD comprises treatment of fibrosis and/or cirrhosis, e.g., a decrease in the severity of fibrosis, a lack of further progression of fibrosis and/or cirrhosis, or a slowing of the progression of fibrosis and/or cirrhosis. In some embodiments, the presence of fibrosis and/or cirrhosis is determined by one or more methods selected from the group consisting of transient elastography (e.g., FIBROSCAN®), non-invasive markers of hepatic fibrosis, and histological features of a liver biopsy. In some embodiments, the severity (e.g., stage) of fibrosis is determined by one or more methods selected from the group consisting of transient elastography (e.g., FIBROSCAN®), a fibrosis-scoring system, biomarkers of hepatic fibrosis (e.g., non-invasive biomarkers), and hepatic venous pressure gradient (HVPG). Non-limiting examples of fibrosis scoring systems include the NAFLD fibrosis scoring system (see, e.g., Angulo, et al., Hepatology. 2007; 45(4):846-54), the fibrosis scoring system in Brunt et al., Am J Gastroenterol. 1999, 94:2467-2474, the fibrosis scoring system in Kleiner et al., Hepatology. 2005, 41(6):1313-1321, and the ISHAK fibrosis scoring system (see Ishak et al., J Hepatol. 1995; 22:696-9), the contents of each of which are incorporated by reference herein in their entireties.

In some embodiments, the severity of fibrosis is determined prior to administration of the combination of (a) the compound of Formula (I), or a pharmaceutically acceptable salt or solvate thereof, and (b) an SGLT-2 inhibitor or a GLP-1 receptor agonist. In some embodiments, the severity of fibrosis is determined prior to administration of the combination of (a) the compound of Formula (I), or a pharmaceutically acceptable salt or solvate thereof, (b) an SGLT-2 inhibitor, and (c) a GLP-1 receptor agonist. In other embodiments, the severity of fibrosis is determined prior to administration of the combination of (a) the compound of Formula (I), or a pharmaceutically acceptable salt or solvate thereof and (b) an SGLT-2 inhibitor. In still other embodiments, the severity of fibrosis is determined prior to administration of the combination of (a) the compound of Formula (I), or a pharmaceutically acceptable salt or solvate thereof and (c) a GLP-1 receptor agonist. In some embodiments, the severity of fibrosis is determined prior to administration of the combination of (a) the compound of Formula (I), or a pharmaceutically acceptable salt or solvate thereof, (b) an SGLT-2 inhibitor, (c) a GLP-1 receptor agonist, and (d) metformin. In other embodiments, the severity of fibrosis is determined prior to administration of the combination of (a) the compound of Formula (I), or a pharmaceutically acceptable salt or solvate thereof, (b) an SGLT-2 inhibitor, and (d) metformin. In still other embodiments, the severity of fibrosis is determined prior to administration of the combination of (a) the compound of Formula (I), or a pharmaceutically acceptable salt or solvate thereof, (c) a GLP-1 receptor agonist, and (d) metformin. In some embodiments, the severity of fibrosis is determined during the period of time or after the period of time of administration of the combination of (a) and (b), the combination of (a), (b), and (c), the combination of (a) and (c), the combination of (a), (b), and (d), the combination of (a), (c), and (d), or the combination of (a), (b), (c), and (d). In some embodiments, a decrease in the severity of fibrosis during the period of time or after the period of time of administration of the combination of (a) and (b), the combination of (a), (b), and (c), the combination of (a) and (c), the combination of (a), (b), and (d), the combination of (a), (c), and (d), or the combination of (a), (b), (c), and (d) compared to prior to administration of the combination of (a) and (b), the combination of (a), (b), and (c), the combination of (a) and (c), the combination of (a), (b), and (d), the combination of (a), (c), and (d), or the combination of (a), (b), (c), and (d) indicates treatment of NAFLD. In some embodiments, a decrease in the severity of fibrosis, a lack of further progression of fibrosis and/or cirrhosis, or a slowing of the progression of fibrosis and/or cirrhosis indicates treatment of NAFLD. In some embodiments, the severity of fibrosis is determined using a scoring system such as any of the fibrosis scoring systems described herein, for example, the score can indicate the stage of fibrosis, e.g., stage 0 (no fibrosis), stage 1, stage 2, stage 3, and stage 4 (cirrhosis) (see, e.g., Kleiner et al). In some embodiments, a decrease in the stage of the fibrosis is a decrease in the severity of the fibrosis. For example, a decrease by 1, 2, 3, or 4 stages is a decrease in the severity of the fibrosis. In some embodiments, a decrease in the stage, e.g., from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 indicates treatment of NAFLD. In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 following administration of the combination of (a) and (b) or the combination of (a), (b), and (c) compared to prior to administration of the combination of (a) and (b) or the combination of (a), (b), and (c). In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 during the period of time of administration of the combination of (a) and (b) or the combination of (a), (b), and (c) compared to prior to administration of the combination of (a) and (b) or the combination of (a), (b), and (c). In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 after the period of time of administration of the combination of (a) and (b), the combination of (a), (b), and (c), the combination of (a) and (c), the combination of (a), (b), and (d), the combination of (a), (c), and (d), or the combination of (a), (b), (c), and (d) compared to prior to administration of the combination of (a) and (b), the combination of (a), (b), and (c), the combination of (a) and (c), the combination of (a), (b), and (d), the combination of (a), (c), and (d), or the combination of (a), (b), (c), and (d).

In some embodiments, the presence of NAFLD is determined by one or more biomarkers indicative of one or more of liver damage, inflammation, liver fibrosis, and/or liver cirrhosis or scoring systems thereof. In some embodiments, the severity of NAFLD is determined by one or more biomarkers indicative of one or more of liver damage, inflammation, liver fibrosis, and/or liver cirrhosis or scoring systems thereof. The level of the biomarker can be determined by, for example, measuring, quantifying, and monitoring the expression level of the gene or mRNA encoding the biomarker and/or the peptide or protein of the biomarker. Non-limiting examples of biomarkers indicative of one or more of liver damage, inflammation, liver fibrosis, and/or liver cirrhosis and/or scoring systems thereof include the aspartate aminotransferase (AST) to platelet ratio index (APRI); the aspartate aminotransferase (AST) and alanine aminotransferase (ALT) ratio (AAR); the FIB-4 score, which is based on the APRI, alanine aminotransferase (ALT) levels, and age of the subject (see, e.g., McPherson et al., Gut. 2010 September; 59(9):1265-9, which is incorporated by reference herein in its entirety); hyaluronic acid; pro-inflammatory cytokines; a panel of biomarkers consisting of a2-macroglobulin, haptoglobin, apolipoprotein A1, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, a2-macroglobulin combined with the subject's age and sex (e.g., HEPASCORE®; see, e.g., Adams et al., Clin Chem. 2005 October; 51(10):1867-73), and a panel of biomarkers consisting of tissue inhibitor of metalloproteinase-1, hyaluronic acid, and a2-macroglobulin (e.g., FIBROSPECT®); a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score, see, e.g., Lichtinghagen R, et al., J Hepatol. 2013 August; 59(2):236-42, which is incorporated by reference herein in its entirety). In some embodiments, the presence of fibrosis is determined by one or more of the FIB-4 score, a panel of biomarkers consisting of α2-macroglobulin, haptoglobin, apolipoprotein A1, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, α2-macroglobulin combined with the subject's age and sex (e.g., HEPASCORE®; see, e.g., Adams et al., Clin Chem. 2005 October; 51(10):1867-73), and a panel of biomarkers consisting of tissue inhibitor of metalloproteinase-1, hyaluronic acid, and α2-macroglobulin (e.g., FIBROSPECT®); and a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score).

In some embodiments, the level of aspartate aminotransferase (AST) does not increase. In some embodiments, the level of aspartate aminotransferase (AST) decreases. In some embodiments, the level of alanine aminotransferase (ALT) does not increase. In some embodiments, the level of alanine aminotransferase (ALT) decreases. In some embodiments, the “level” of an enzyme refers to the concentration of the enzyme, e.g., within blood. For example, the level of AST or ALT can be expressed as Units/L.

In some embodiments, the severity of fibrosis is determined by one or more of the FIB-4 score, a panel of biomarkers consisting of α2-macroglobulin, haptoglobin, apolipoprotein A1, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, α2-macroglobulin combined with the subject's age and sex (e.g., HEPASCORE®; see, e.g., Adams et al., Clin Chem. 2005 October; 51(10):1867-73, which is incorporated by reference herein in its entirety), and a panel of biomarkers consisting of tissue inhibitor of metalloproteinase-1, hyaluronic acid, and α2-macroglobulin (e.g., FIBROSPECT®); and a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score).

In some embodiments, hepatic inflammation is determined by the level of liver inflammation biomarkers, e.g., pro-inflammatory cytokines. Non-limiting examples of biomarkers indicative of liver inflammation include interleukin-(IL) 6, interleukin-(IL) 1β, tumor necrosis factor (TNF)-α, transforming growth factor (TGF)-β, monocyte chemotactic protein (MCP)-1, C-reactive protein (CRP), PAI-1, and collagen isoforms such as Col1a1, Col1a2, and Col4a1 (see, e.g., Neuman, et al., Can J Gastroenterol Hepatol. 2014 December; 28(11): 607-618 and U.S. Pat. No. 9,872,844, each of which are incorporated by reference herein in their entireties). Liver inflammation can also be assessed by change of macrophage infiltration, e.g., measuring a change of CD68 expression level. In some embodiments, liver inflammation can be determined by measuring or monitoring serum levels or circulating levels of one or more of interleukin-(IL) 6, interleukin-(IL) 1β, tumor necrosis factor (TNF)-α, transforming growth factor (TGF)-β, monocyte chemotactic protein (MCP)-1, and C-reactive protein (CRP).

In some embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt or solvate thereof, and (b) an SGLT-2 inhibitor or a GLP-1 receptor agonist. In some embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt or solvate thereof, (b) an SGLT-2 inhibitor, and (c) a GLP-1 receptor agonist. In other embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt or solvate thereof and (b) an SGLT-2 inhibitor. In still other embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt or solvate thereof and (c) a GLP-1 receptor agonist. In some embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt or solvate thereof, (b) an SGLT-2 inhibitor, (c) a GLP-1 receptor agonist, and (d) metformin. In other embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt or solvate thereof, (b) an SGLT-2 inhibitor, and (d) metformin. In still other embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt or solvate thereof, (c) a GLP-1 receptor agonist, and (d) metformin. In some embodiments, the level of one or more biomarkers indicative of one or more of liver damage, inflammation, liver fibrosis, and/or liver cirrhosis is determined during the period of time or after the period of time of administration of the combination of (a) and (b), the combination of (a), (b), and (c), the combination of (a) and (c), the combination of (a), (b), and (d), the combination of (a), (c), and (d), or the combination of (a), (b), (c), and (d). In some embodiments, a decrease in the level of one or more biomarkers indicative of one or more of liver damage, inflammation, liver fibrosis, and/or liver cirrhosis during the period of time or after the period of time of administration of the combination of (a) and (b), the combination of (a), (b), and (c), the combination of (a) and (c), the combination of (a), (b), and (d), the combination of (a), (c), and (d), or the combination of (a), (b), (c), and (d) compared to prior to administration of the combination of (a) and (b), the combination of (a), (b), and (c), the combination of (a) and (c), the combination of (a), (b), and (d), the combination of (a), (c), and (d), or the combination of (a), (b), (c), and (d) indicates treatment of NAFLD. For example, a decrease in the level of one or more biomarkers indicative of one or more of liver damage, inflammation, liver fibrosis, and/or liver cirrhosis by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% indicates treatment of NAFLD. In some embodiments, the decrease in the level of one or more biomarkers indicative of one or more of liver damage, inflammation, liver fibrosis, and/or liver cirrhosis following administration of the combination of (a) and (b), the combination of (a), (b), and (c), the combination of (a) and (c), the combination of (a), (b), and (d), the combination of (a), (c), and (d), or the combination of (a), (b), (c), and (d) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%. In some embodiments, the level of one or more biomarkers indicative of one or more of liver damage, inflammation, liver fibrosis, and/or liver cirrhosis during the period of time of administration of the combination of (a) and (b), the combination of (a), (b), and (c), the combination of (a) and (c), the combination of (a), (b), and (d), the combination of (a), (c), and (d), or the combination of (a), (b), (c), and (d) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%. In some embodiments, the level of one or more biomarkers indicative of one or more of liver damage, inflammation, liver fibrosis, and/or liver cirrhosis after the period of time of administration of the combination of (a) and (b), the combination of (a), (b), and (c), the combination of (a) and (c), the combination of (a), (b), and (d), the combination of (a), (c), and (d), or the combination of (a), (b), (c), and (d) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

In some embodiments, the treatment of NAFLD decreases the level of serum bile acids in the subject. In some embodiments, the level of serum bile acids is determined by, for example, an ELISA enzymatic assay or the assays for the measurement of total bile acids as described in Danese et al., PLoS One. 2017; 12(6): e0179200, which is incorporated by reference herein in its entirety. In some embodiments, the level of serum bile acids can decrease by, for example, 10% to 40%, 20% to 50%, 30% to 60%, 40% to 70%, 50% to 80%, or by more than 90% of the level of serum bile acids prior to administration of the combination of (a) and (b), the combination of (a), (b), and (c), the combination of (a) and (c), the combination of (a), (b), and (d), the combination of (a), (c), and (d), or the combination of (a), (b), (c), and (d). In some embodiments, the NAFLD is NAFLD with attendant cholestasis. In cholestasis, the release of bile, including bile acids, from the liver is blocked. Bile acids can cause hepatocyte damage (see, e.g., Perez M J, Briz O. World J Gastroenterol. 2009 Apr. 14; 15(14):1677-89) likely leading to or increasing the progression of fibrosis (e.g., cirrhosis) and increasing the risk of hepatocellular carcinoma (see, e.g., Sorrentino P et al. Dig Dis Sci. 2005 June; 50(6):1130-5 and Satapathy S K and Sanyal A J. Semin Liver Dis. 2015, 35(3):221-35, each of which are incorporated by reference herein in their entireties). In some embodiments, the NAFLD with attendant cholestasis is NASH with attendant cholestasis. In some embodiments, the treatment of NAFLD comprises treatment of pruritus. In some embodiments, the treatment of NAFLD with attendant cholestasis comprises treatment of pruritus. In some embodiments, a subject with NAFLD with attendant cholestasis has pruritus.

In some embodiments, treatment of NAFLD comprises an increase in adiponectin. It is thought that the compound of Formula (I) may be a selective activator of a highly limited number of PPARγ pathways including pathways regulated by adiponectin. Adiponectin is an anti-fibrotic and anti-inflammatory adipokine in the liver (see e.g., Park et al., Curr Pathobiol Rep. 2015 Dec. 1; 3(4): 243-252). In some embodiments, the level of adiponectin is determined by, for example, an ELISA enzymatic assay. In some embodiments, the adiponectin level in the subject is increased by at least about 30%, at least about 68%, at least about 175%, or at least about 200%. In some embodiments, the increase is by at least about 175%. In some embodiments, the level of adiponectin is determined for a sample from the subject prior to administration of the combination of (a) the compound of Formula (I), or a pharmaceutically acceptable salt or solvate thereof, and (b) an SGLT-2 inhibitor or a GLP-1 receptor agonist. In some embodiments, the level of adiponectin is determined for a sample from the subject prior to administration of the combination of (a) the compound of Formula (I), or a pharmaceutically acceptable salt or solvate thereof, (b) an SGLT-2 inhibitor, and (c) a GLP-1 receptor agonist. In other embodiments, the level of adiponectin is determined for a sample from the subject prior to administration of the combination of (a) the compound of Formula (I), or a pharmaceutically acceptable salt or solvate thereof and (b) an SGLT-2 inhibitor. In still other embodiments, the level of adiponectin is determined for a sample from the subject prior to administration of the combination of (a) the compound of Formula (I), or a pharmaceutically acceptable salt or solvate thereof and (c) a GLP-1 receptor agonist. In some embodiments, the level of adiponectin is determined for a sample from the subject prior to administration of the combination of (a) the compound of Formula (I), or a pharmaceutically acceptable salt or solvate thereof, (b) an SGLT-2 inhibitor, (c) a GLP-1 receptor agonist, and (d) metformin. In other embodiments, the level of adiponectin is determined for a sample from the subject prior to administration of the combination of (a) the compound of Formula (I), or a pharmaceutically acceptable salt or solvate thereof, (b) an SGLT-2 inhibitor, and (d) metformin. In still other embodiments, the level of adiponectin is determined for a sample from the subject prior to administration of the combination of (a) the compound of Formula (I), or a pharmaceutically acceptable salt or solvate thereof, (c) a GLP-1 receptor agonist, and (d) metformin. In some embodiments, the level of adiponectin is determined during the period of time or after the period of time of administration of the combination of (a) and (b), the combination of (a), (b), and (c), the combination of (a) and (c), the combination of (a), (b), and (d), the combination of (a), (c), and (d), or the combination of (a), (b), (c), and (d). In some embodiments, an increase in the level of adiponectin during the period of time or after the period of time of administration of the combination of (a) and (b), the combination of (a), (b), and (c), the combination of (a) and (c), the combination of (a), (b), and (d), the combination of (a), (c), and (d), or the combination of (a), (b), (c), and (d) compared to prior to administration of the combination of (a) and (b), the combination of (a), (b), and (c), the combination of (a) and (c), the combination of (a), (b), and (d), the combination of (a), (c), and (d), or the combination of (a), (b), (c), and (d) indicates treatment of NAFLD. For example, an increase in the level of adiponectin by at least about 30%, at least about 68%, at least about 175%, or at least about 200% indicates treatment of NAFLD. In some embodiments, the increase in the level of adiponectin following administration of the combination of (a) and (b), the combination of (a), (b), and (c), the combination of (a) and (c), the combination of (a), (b), and (d), the combination of (a), (c), and (d), or the combination of (a), (b), (c), and (d) is at least about 200%.

Provided herein are methods of treating non-alcoholic fatty liver disease (NAFLD) in a subject in need thereof comprising or consisting essentially of administering to the subject (a) the compound of Formula (I), or a pharmaceutically acceptable salt or solvate thereof, and (b) an SGLT-2 inhibitor, or a pharmaceutically acceptable salt or solvate thereof, wherein the amounts of (a) and (b) together are effective in treating NAFLD. In some embodiments, a method of treating non-alcoholic fatty liver disease (NAFLD) in a subject in need thereof comprises or consists essentially of administering to the subject (a) the compound of Formula (I), or a pharmaceutically acceptable salt or solvate thereof, and (b) an SGLT-2 inhibitor, or a pharmaceutically acceptable salt or solvate thereof, during a period of time, wherein the amounts of (a) and (b) together are effective in treating NAFLD.

Also provided herein are methods of treating a subject, the method comprising: selecting a subject having non-alcoholic fatty liver disease (NAFLD); and administering (a) the compound of Formula (I), or a pharmaceutically acceptable salt or solvate thereof, and (b) an SGLT-2 inhibitor, or a pharmaceutically acceptable salt or solvate thereof, to the selected subject, wherein the amounts of (a) and (b) together are effective in treating NAFLD. In some embodiments, (a) and (b) are administered during a period of time.

Also provided herein are methods of treating a subject, the method comprising: identifying a subject having non-alcoholic fatty liver disease (NAFLD); and administering (a) the compound of Formula (I), or a pharmaceutically acceptable salt or solvate thereof, and (b) an SGLT-2 inhibitor, or a pharmaceutically acceptable salt or solvate thereof, to the selected subject, wherein the amounts of (a) and (b) together are effective in treating NAFLD. In some embodiments, (a) and (b) are administered during a period of time.

Provided herein are methods of treating NAFLD in a subject in need thereof comprising or consisting essentially of administering to the subject (a) a therapeutically effective amount of the compound of Formula (I), or a pharmaceutically acceptable salt or solvate thereof, and (b) a therapeutically effective amount of an SGLT-2 inhibitor, or a pharmaceutically acceptable salt or solvate thereof. In some embodiments, a method of treating NAFLD in a subject in need thereof comprises or consists essentially of administering to the subject (a) a therapeutically effective amount of the compound of Formula (I), or a pharmaceutically acceptable salt or solvate thereof, and (b) a therapeutically effective amount of an SGLT-2 inhibitor, or a pharmaceutically acceptable salt or solvate thereof, during a period of time. In some embodiments, the amounts of (a) and (b) together are effective in treating NAFLD.

Also provided herein are methods of treating a subject, the method comprising: selecting a subject having non-alcoholic fatty liver disease (NAFLD); and administering (a) a therapeutically effective amount of the compound of Formula (I), or a pharmaceutically acceptable salt or solvate thereof, and (b) a therapeutically effective amount of an SGLT-2 inhibitor, or a pharmaceutically acceptable salt or solvate thereof, to the selected subject. In some embodiments, (a) and (b) are administered during a period of time. In some embodiments, the amounts of (a) and (b) together are effective in treating NAFLD.

Also provided here are methods of selecting a subject for treatment, the method comprising: identifying a subject having NAFLD; and selecting the identified subject for treatment with (a) a therapeutically effective amount of the compound of Formula (I), or a pharmaceutically acceptable salt or solvate thereof, and (b) a therapeutically effective amount of an SGLT-2 inhibitor, or a pharmaceutically acceptable salt or solvate thereof. In some embodiments, the amounts of (a) and (b) together are effective in treating NAFLD.

Also provided herein are methods of selecting a subject for participation in a clinical trial, the method comprising: identifying a subject having NAFLD; and selecting the identified subject for participation in a clinical trial that comprises administration of (a) a therapeutically effective amount of the compound of Formula (I), or a pharmaceutically acceptable salt or solvate thereof, and (b) a therapeutically effective amount of an SGLT-2 inhibitor, or a pharmaceutically acceptable salt or solvate thereof. In some embodiments, the amounts of (a) and (b) together are effective in treating NAFLD.

In some embodiments, (a) and (b) are administered concurrently. In some embodiments, (a) and (b) are administered as a fixed combination. In some embodiments, (a) and (b) are administered as a non-fixed combination. In some embodiments, (a) and (b) are administered sequentially and in any order, at specific or varying time intervals (e.g., during the period of time). In some embodiments, a therapeutically effective amount of each of (a) and (b) are administered concurrently. In some embodiments, a therapeutically effective amount of each of (a) and (b) are administered sequentially and in any order, at specific or varying time intervals (e.g., during the period of time).

In some embodiments, the amount of the compound of Formula (I), or a pharmaceutically acceptable salt or solvate thereof, is from about 0.1 to about 15 milligrams (mg). For example, from about 0.1 to about 10 mg, about 5 to about 15 mg, or about 2 to about 12 mg. In some embodiments, the compound of Formula (I), or a pharmaceutically acceptable salt or solvate thereof, is administered at a dose from about 0.1 to about 5 mg, about 0.1 to about 4 mg, about 0.5 to about 3 mg, about 0.5 to about 2 mg, about 0.5 to about 1 mg, about 1 to about 3 mg, about 2 to about 4 mg, about 3 to about 5 mg, about 1 to about 6 mg, about 2 to about 6 mg, about 3 to about 6 mg, about 4 to about 6 mg, or about 5 to about 6 mg. For example, about 0.10 mg, about 0.15 mg, about 0.20 mg, about 0.25 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1.00 mg, about 1.05 mg, about 1.10 mg, about 1.15 mg, about 1.20 mg, about 1.25 mg, about 1.30 mg, about 1.35 mg, about 1.40 mg, about 1.45 mg, about 1.50 mg, about 1.55 mg, about 1.60 mg, about 1.65 mg, about 1.70 mg, about 1.75 mg, about 1.80 mg, about 1.85 mg, about 1.90 mg, about 1.95 mg, about 2.00 mg, about 2.05 mg, about 2.10 mg, about 2.15 mg, about 2.20 mg, about 2.25 mg, about 2.30 mg, about 2.35 mg, about 2.40 mg, about 2.45 mg, about 2.50 mg, about 2.55 mg, about 2.60 mg, about 2.65 mg, about 2.70 mg, about 2.75 mg, about 2.80 mg, about 2.85 mg, about 2.90 mg, about 2.95 mg, about 3.00 mg, about 3.05 mg, about 3.10 mg, about 3.15 mg, about 3.20 mg, about 3.25 mg, about 3.30 mg, about 3.35 mg, about 3.40 mg, about 3.45 mg, about 3.50 mg, about 3.55 mg, about 3.60 mg, about 3.65 mg, about 3.70 mg, about 3.75 mg, about 3.80 mg, about 3.85 mg, about 3.90 mg, about 3.95 mg, about 4.00 mg, about 4.05 mg, about 4.10 mg, about 4.15 mg, about 4.20 mg, about 4.25 mg, about 4.30 mg, about 4.35 mg, about 4.40 mg, about 4.45 mg, about 4.50 mg, about 4.55 mg, about 4.60 mg, about 4.65 mg, about 4.70 mg, about 4.75 mg, about 4.80 mg, about 4.85 mg, about 4.90 mg, about 4.95 mg, about 5.00 mg, about 5.05 mg, about 5.10 mg, about 5.15 mg, about 5.20 mg, about 5.25 mg, about 5.30 mg, about 5.35 mg, about 5.40 mg, about 5.45 mg, about 5.50 mg, about 5.55 mg, about 5.60 mg, about 5.65 mg, about 5.70 mg, about 5.75 mg, about 5.80 mg, about 5.85 mg, about 5.90 mg, about 5.95 mg, about 6.00 mg, about 6.05 mg, about 6.10 mg, about 6.15 mg, about 6.20 mg, about 6.25 mg, about 6.30 mg, about 6.35 mg, about 6.40 mg, about 6.45 mg, about 6.50 mg, about 6.55 mg, about 6.60 mg, about 6.65 mg, about 6.70 mg, about 6.75 mg, about 6.80 mg, about 6.85 mg, about 6.90 mg, about 6.95 mg, about 7.00 mg, about 7.05 mg, about 7.10 mg, about 7.15 mg, about 7.20 mg, about 7.25 mg, about 7.30 mg, about 7.35 mg, about 7.40 mg, about 7.45 mg, about 7.50 mg, about 7.55 mg, about 7.60 mg, about 7.65 mg, about 7.70 mg, about 7.75 mg, about 7.80 mg, about 7.85 mg, about 7.90 mg, about 7.95 mg, about 8.00 mg, about 8.05 mg, about 8.10 mg, about 8.15 mg, about 8.20 mg, about 8.25 mg, about 8.30 mg, about 8.35 mg, about 8.40 mg, about 8.45 mg, about 8.50 mg, about 8.55 mg, about 8.60 mg, about 8.65 mg, about 8.70 mg, about 8.75 mg, about 8.80 mg, about 8.85 mg, about 8.90 mg, about 8.95 mg, about 9.00 mg, about 9.05 mg, about 9.10 mg, about 9.15 mg, about 9.20 mg, about 9.25 mg, about 9.30 mg, about 9.35 mg, about 9.40 mg, about 9.45 mg, about 9.50 mg, about 9.55 mg, about 9.60 mg, about 9.65 mg, about 9.70 mg, about 9.75 mg, about 9.80 mg, about 9.85 mg, about 9.90 mg, about 9.95 mg, or about 10.00 mg. In some embodiments, the dose is a therapeutically effective amount.

In some embodiments, the compound of Formula (I), or a pharmaceutically acceptable salt or solvate thereof, is administered at a dose from about 0.1 to about 15 mg. For example, from about 0.1 to about 10 mg, about 5 to about 15 mg, or about 2 to about 12 mg. In some embodiments, the compound of Formula (I), or a pharmaceutically acceptable salt or solvate thereof, is administered at a dose from about 0.1 to about 5 mg, about 0.1 to about 4 mg, about 0.5 to about 3 mg, about 0.5 to about 2 mg, about 0.5 to about 1 mg, about 1 to about 3 mg, about 2 to about 4 mg, about 3 to about 5 mg, about 1 to about 6 mg, about 2 to about 6 mg, about 3 to about 6 mg, about 4 to about 6 mg, or about 5 to about 6 mg. For example, about 0.10 mg, about 0.15 mg, about 0.20 mg, about 0.25 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1.00 mg, about 1.05 mg, about 1.10 mg, about 1.15 mg, about 1.20 mg, about 1.25 mg, about 1.30 mg, about 1.35 mg, about 1.40 mg, about 1.45 mg, about 1.50 mg, about 1.55 mg, about 1.60 mg, about 1.65 mg, about 1.70 mg, about 1.75 mg, about 1.80 mg, about 1.85 mg, about 1.90 mg, about 1.95 mg, about 2.00 mg, about 2.05 mg, about 2.10 mg, about 2.15 mg, about 2.20 mg, about 2.25 mg, about 2.30 mg, about 2.35 mg, about 2.40 mg, about 2.45 mg, about 2.50 mg, about 2.55 mg, about 2.60 mg, about 2.65 mg, about 2.70 mg, about 2.75 mg, about 2.80 mg, about 2.85 mg, about 2.90 mg, about 2.95 mg, about 3.00 mg, about 3.05 mg, about 3.10 mg, about 3.15 mg, about 3.20 mg, about 3.25 mg, about 3.30 mg, about 3.35 mg, about 3.40 mg, about 3.45 mg, about 3.50 mg, about 3.55 mg, about 3.60 mg, about 3.65 mg, about 3.70 mg, about 3.75 mg, about 3.80 mg, about 3.85 mg, about 3.90 mg, about 3.95 mg, about 4.00 mg, about 4.05 mg, about 4.10 mg, about 4.15 mg, about 4.20 mg, about 4.25 mg, about 4.30 mg, about 4.35 mg, about 4.40 mg, about 4.45 mg, about 4.50 mg, about 4.55 mg, about 4.60 mg, about 4.65 mg, about 4.70 mg, about 4.75 mg, about 4.80 mg, about 4.85 mg, about 4.90 mg, about 4.95 mg, about 5.00 mg, about 5.05 mg, about 5.10 mg, about 5.15 mg, about 5.20 mg, about 5.25 mg, about 5.30 mg, about 5.35 mg, about 5.40 mg, about 5.45 mg, about 5.50 mg, about 5.55 mg, about 5.60 mg, about 5.65 mg, about 5.70 mg, about 5.75 mg, about 5.80 mg, about 5.85 mg, about 5.90 mg, about 5.95 mg, about 6.00 mg, about 6.05 mg, about 6.10 mg, about 6.15 mg, about 6.20 mg, about 6.25 mg, about 6.30 mg, about 6.35 mg, about 6.40 mg, about 6.45 mg, about 6.50 mg, about 6.55 mg, about 6.60 mg, about 6.65 mg, about 6.70 mg, about 6.75 mg, about 6.80 mg, about 6.85 mg, about 6.90 mg, about 6.95 mg, about 7.00 mg, about 7.05 mg, about 7.10 mg, about 7.15 mg, about 7.20 mg, about 7.25 mg, about 7.30 mg, about 7.35 mg, about 7.40 mg, about 7.45 mg, about 7.50 mg, about 7.55 mg, about 7.60 mg, about 7.65 mg, about 7.70 mg, about 7.75 mg, about 7.80 mg, about 7.85 mg, about 7.90 mg, about 7.95 mg, about 8.00 mg, about 8.05 mg, about 8.10 mg, about 8.15 mg, about 8.20 mg, about 8.25 mg, about 8.30 mg, about 8.35 mg, about 8.40 mg, about 8.45 mg, about 8.50 mg, about 8.55 mg, about 8.60 mg, about 8.65 mg, about 8.70 mg, about 8.75 mg, about 8.80 mg, about 8.85 mg, about 8.90 mg, about 8.95 mg, about 9.00 mg, about 9.05 mg, about 9.10 mg, about 9.15 mg, about 9.20 mg, about 9.25 mg, about 9.30 mg, about 9.35 mg, about 9.40 mg, about 9.45 mg, about 9.50 mg, about 9.55 mg, about 9.60 mg, about 9.65 mg, about 9.70 mg, about 9.75 mg, about 9.80 mg, about 9.85 mg, about 9.90 mg, about 9.95 mg, or about 10.00 mg. In some embodiments, the dose is a therapeutically effective amount.

In some embodiments, the compound of Formula (I), or a pharmaceutically acceptable salt or solvate thereof, is administered to the subject twice a day, daily, every other day, three times a week, twice a week, weekly, every other week, twice a month, or monthly. In some embodiments, the compound of Formula (I), or a pharmaceutically acceptable salt or solvate thereof, is administered to the subject daily.

In some embodiments, the compound of Formula (I), or a pharmaceutically acceptable salt or solvate thereof, is administered to the subject daily and at a dose of about 3 mg. In some embodiments, the compound of Formula (I), or a pharmaceutically acceptable salt or solvate thereof, is administered at a dose from about 0.1 to about 10.0 mg per day. In some embodiments, the compound of Formula (I), or a pharmaceutically acceptable salt or solvate thereof, is administered at a dose from about 0.1 to about 3 mg per day. In some embodiments, the compound of Formula (I), or a pharmaceutically acceptable salt or solvate thereof, is administered at a dose about 0.5 milligram per day. In some embodiments, the compound of Formula (I), or a pharmaceutically acceptable salt or solvate thereof, is administered at a dose about 1 milligram per day. In some embodiments, the compound of Formula (I), or a pharmaceutically acceptable salt or solvate thereof, is administered at a dose about 2 mg per day.

In some embodiments, the compound of Formula (I) is in the form of a besylate salt. In some embodiments, the compound of Formula (I) is in the form of an HCl salt. In some embodiments, the compound of Formula (I) is in the form of an HBr salt. In some embodiments, the compound of Formula (I) is in the form of a tosylate salt.

In some embodiments described herein, the SGLT-2 inhibitor is selected from the group consisting of: empagliflozin, canagliflozin, dapagliflozin, ertugliflozin, ipragliflozin, luseogliflozin, remogliflozin etabonate, serfliflozin etabonate, sotagliflozin, tofogliflozin, or a pharmaceutically acceptable salt, solvate, and/or combination of two or more thereof. In some embodiments, the SGLT-2 inhibitor is empagliflozin. In some embodiments, the SGLT-2 inhibitor is empagliflozin. In other embodiments, the SGLT-2 inhibitor is dapagliflozin. In some embodiments, the SGLT-2 inhibitor is dapagliflozin propylene glycol hydrate. In still other embodiments, the SGLT-2 inhibitor is canagliflozin. In some embodiments, the SGLT-2 inhibitor is canagliflozin hemihydrate.

In some embodiments, the amount of the SGLT-2 inhibitor, or a pharmaceutically acceptable salt or solvate thereof, is from about 1 to about 350 mg. For example, about 1 to about 175 mg, about 175 to about 350 mg, or about 90 to about 260 mg. In some embodiments, the amount of the SGLT-2 inhibitor, or a pharmaceutically acceptable salt or solvate thereof, is from about 85 to about 325 mg. In some embodiments, the amount of the SGLT-2 inhibitor, or a pharmaceutically acceptable salt or solvate thereof, is from about 1 to about 50 mg, about 20 to about 70 mg, about 50 to about 100 mg, about 70 to about 120 mg, about 90 to about 140 mg, about 110 to about 160 mg, about 130 to about 180 mg, about 150 to about 200 mg, about 170 to about 220 mg, about 190 to about 240 mg, about 210 to about 260 mg, about 230 to about 280 mg, about 250 to about 300 mg, about 270 to about 320 mg, or about 290 to about 350 mg. For example, about 100 mg or about 300 mg. In some embodiments, the amount of the SGLT-2 inhibitor, or a pharmaceutically acceptable salt or solvate thereof, is from about 1 to about 15 mg. For example, about 1 to about 10 mg or about 5 to about 15 mg. In some embodiments, the amount of the SGLT-2 inhibitor, or a pharmaceutically acceptable salt or solvate thereof, is from 1 to about 3 mg, about 2 to about 4 mg, about 3 to about 5 mg, about 4 to about 6 mg, about 5 to about 7, about 6 to about 8, about 7 to about 9 mg, about 8 to about 10 mg, about 9 to about 11 mg, about 10 to about 12 mg, about 11 to about 13 mg, about 12 to about 14 mg, or about 13 to about 15 mg. For example, about 0.10 mg, about 0.15 mg, about 0.20 mg, about 0.25 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1.00 mg, about 1.05 mg, about 1.10 mg, about 1.15 mg, about 1.20 mg, about 1.25 mg, about 1.30 mg, about 1.35 mg, about 1.40 mg, about 1.45 mg, about 1.50 mg, about 1.55 mg, about 1.60 mg, about 1.65 mg, about 1.70 mg, about 1.75 mg, about 1.80 mg, about 1.85 mg, about 1.90 mg, about 1.95 mg, about 2.00 mg, about 2.05 mg, about 2.10 mg, about 2.15 mg, about 2.20 mg, about 2.25 mg, about 2.30 mg, about 2.35 mg, about 2.40 mg, about 2.45 mg, about 2.50 mg, about 2.55 mg, about 2.60 mg, about 2.65 mg, about 2.70 mg, about 2.75 mg, about 2.80 mg, about 2.85 mg, about 2.90 mg, about 2.95 mg, about 3.00 mg, about 3.05 mg, about 3.10 mg, about 3.15 mg, about 3.20 mg, about 3.25 mg, about 3.30 mg, about 3.35 mg, about 3.40 mg, about 3.45 mg, about 3.50 mg, about 3.55 mg, about 3.60 mg, about 3.65 mg, about 3.70 mg, about 3.75 mg, about 3.80 mg, about 3.85 mg, about 3.90 mg, about 3.95 mg, about 4.00 mg, about 4.05 mg, about 4.10 mg, about 4.15 mg, about 4.20 mg, about 4.25 mg, about 4.30 mg, about 4.35 mg, about 4.40 mg, about 4.45 mg, about 4.50 mg, about 4.55 mg, about 4.60 mg, about 4.65 mg, about 4.70 mg, about 4.75 mg, about 4.80 mg, about 4.85 mg, about 4.90 mg, about 4.95 mg, about 5.00 mg, about 5.05 mg, about 5.10 mg, about 5.15 mg, about 5.20 mg, about 5.25 mg, about 5.30 mg, about 5.35 mg, about 5.40 mg, about 5.45 mg, about 5.50 mg, about 5.55 mg, about 5.60 mg, about 5.65 mg, about 5.70 mg, about 5.75 mg, about 5.80 mg, about 5.85 mg, about 5.90 mg, about 5.95 mg, about 6.00 mg, about 6.05 mg, about 6.10 mg, about 6.15 mg, about 6.20 mg, about 6.25 mg, about 6.30 mg, about 6.35 mg, about 6.40 mg, about 6.45 mg, about 6.50 mg, about 6.55 mg, about 6.60 mg, about 6.65 mg, about 6.70 mg, about 6.75 mg, about 6.80 mg, about 6.85 mg, about 6.90 mg, about 6.95 mg, about 7.00 mg, about 7.05 mg, about 7.10 mg, about 7.15 mg, about 7.20 mg, about 7.25 mg, about 7.30 mg, about 7.35 mg, about 7.40 mg, about 7.45 mg, about 7.50 mg, about 7.55 mg, about 7.60 mg, about 7.65 mg, about 7.70 mg, about 7.75 mg, about 7.80 mg, about 7.85 mg, about 7.90 mg, about 7.95 mg, about 8.00 mg, about 8.05 mg, about 8.10 mg, about 8.15 mg, about 8.20 mg, about 8.25 mg, about 8.30 mg, about 8.35 mg, about 8.40 mg, about 8.45 mg, about 8.50 mg, about 8.55 mg, about 8.60 mg, about 8.65 mg, about 8.70 mg, about 8.75 mg, about 8.80 mg, about 8.85 mg, about 8.90 mg, about 8.95 mg, about 9.00 mg, about 9.05 mg, about 9.10 mg, about 9.15 mg, about 9.20 mg, about 9.25 mg, about 9.30 mg, about 9.35 mg, about 9.40 mg, about 9.45 mg, about 9.50 mg, about 9.55 mg, about 9.60 mg, about 9.65 mg, about 9.70 mg, about 9.75 mg, about 9.80 mg, about 9.85 mg, about 9.90 mg, about 9.95 mg, about 10.00 mg, about 11 mg, about 12 mg, about 13 mg, or about 15 mg.

In some embodiments, the SGLT-2 inhibitor, or a pharmaceutically acceptable salt or solvate thereof, is administered at a dose from about 1 to about 350 mg. For example, about 1 to about 175 mg, about 175 to about 350 mg, or about 90 to about 260 mg. In some embodiments, the SGLT-2 inhibitor, or a pharmaceutically acceptable salt or solvate thereof, is administered at a dose from about 85 to about 325 mg. In some embodiments, the SGLT-2 inhibitor, or a pharmaceutically acceptable salt or solvate thereof, is administered at a dose from about 1 to about 50 mg, about 20 to about 70 mg, about 50 to about 100 mg, about 70 to about 120 mg, about 90 to about 140 mg, about 110 to about 160 mg, about 130 to about 180 mg, about 150 to about 200 mg, about 170 to about 220 mg, about 190 to about 240 mg, about 210 to about 260 mg, about 230 to about 280 mg, about 250 to about 300 mg, about 270 to about 320 mg, or about 290 to about 350 mg. For example, about 100 mg or about 300 mg. In some embodiments, the SGLT-2 inhibitor, or a pharmaceutically acceptable salt or solvate thereof, is administered at a dose from about 1 to about 15 mg. For example, about 1 to about 10 mg or about 5 to about 15 mg. In some embodiments, the SGLT-2 inhibitor, or a pharmaceutically acceptable salt or solvate thereof, is administered at a dose from 1 to about 3 mg, about 2 to about 4 mg, about 3 to about 5 mg, about 4 to about 6 mg, about 5 to about 7, about 6 to about 8, about 7 to about 9 mg, about 8 to about 10 mg, about 9 to about 11 mg, about 10 to about 12 mg, about 11 to about 13 mg, about 12 to about 14 mg, or about 13 to about 15 mg. For example, about 1 mg, 2 mg, about 3 mg, about 4 mg, about 5 mg, about 6 mg, about 7 mg, about 8 mg, about 9 mg, about 10 mg, about 11 mg, about 12 mg, about 13 mg, or about 15 mg.

In some embodiments, the SGLT-2 inhibitor, or a pharmaceutically acceptable salt or solvate thereof, is administered to the subject twice a day, daily, every other day, three times a week, twice a week, weekly, every other week, twice a month, or monthly. In some embodiments, the SGLT-2 inhibitor, or a pharmaceutically acceptable salt or solvate thereof, is administered to the subject daily.

In some embodiments, the SGLT-2 inhibitor is canagliflozin. In some embodiments, 100 mg or 300 mg of canagliflozin is administered. In some embodiments, 100 mg or 300 mg of canagliflozin hemihydrate is administered. In some other embodiments, the SGLT-2 inhibitor is dapagliflozin. In still other embodiments, the SGLT-2 inhibitor is dapagliflozin propylene glycol hydrate. In some embodiments, 5 mg or 10 mg of dapagliflozin is administered. In still other embodiments, 5 mg or 10 mg of dapagliflozin propylene glycol hydrate is administered. In some embodiments, the SGLT-2 inhibitor is empagliflozin. In some embodiments, 10 mg or 25 mg of empagliflozin is administered. In other embodiments, the SGLT-2 inhibitor is ertugliflozin. In some embodiments, 5 mg or 15 mg of ertugliflozin is administered. In still other embodiments, the SGLT-2 inhibitor is ipragliflozin. In some embodiments, 25 mg or 50 mg of ipragliflozin is administered. In some embodiments, the SGLT-2 inhibitor is bexagliflozin. In some embodiments, 20 mg of bexagliflozin is administered. In other embodiments, the SGLT-2 inhibitor is sotagliflozin. In some embodiments, 200 mg or 400 mg of sotagliflozin is administered. In some embodiments, the SGLT-2 inhibitor is licogliflozin. In some embodiments, 15 mg, 50 mg, 75 mg or 150 mg of licogliflozin is administered.

In some embodiments, the amount of the metformin, or a pharmaceutically acceptable salt or solvate thereof, is from about 250 mg to about 2,500 mg, from about 500 mg to about 2,000 mg, from about 750 mg to about 1,500 mg, from about 1,000 mg to about 1,250 mg, or any value in between. In some embodiments, the metformin is present as the hydrochloride salt.

In some embodiments, the metformin, or a pharmaceutically acceptable salt or solvate thereof, is administered to the subject twice a day, daily, every other day, three times a week, twice a week, weekly, every other week, twice a month, or monthly. In some embodiments, the metformin, or a pharmaceutically acceptable salt or solvate thereof, is administered to the subject daily.

In some embodiments, treatment of NAFLD comprises a decrease of one or more symptoms associated with NAFLD in the subject. Exemplary symptoms can include one or more of an enlarged liver, fatigue, pain in the upper right abdomen, abdominal swelling, enlarged blood vessels just beneath the skin's surface, enlarged breasts in men, enlarged spleen, red palms, jaundice, and pruritus. In some embodiments, the subject is asymptomatic.

In some embodiments, the treatment of NAFLD, e.g., NAFL or NASH, comprises a reduction in hepatic steatosis. For example, hepatic steatosis is decreased by at least 2%, 3%, 4%, 5%, 6%, 7%, 8%. 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 96%, 97%, 98%, 99%, or more than 99% following administration of (a) and (b) for a period of time.

In some embodiments, the treatment of NAFLD, e.g., NAFL or NASH, is assessed using the NAFLD Activity Score (NAS). In some embodiments, treatment of NAFLD comprises a decrease in the NAS. In some embodiments, the NAS for a sample from the subject following administration is 7 or less. In some embodiments, the NAS for a sample from the subject following administration is 5 or less, 4 or less, 3 or less, or 2 or less. In some embodiments, the NAFLD activity score (NAS) for a sample from the subject following administration during the period of time is 7 or less. In some embodiments, the NAS for a sample from the subject following administration during the period of time is 5 or less, 4 or less, 3 or less, or 2 or less. In some embodiments, the sample from the subject is from a liver biopsy.

In some embodiments, the treatment of NAFLD, e.g., NAFL or NASH, can be assessed using the NAFLD Activity Score (NAS). In some embodiments, the NAS for a sample from the subject following administration is reduced by 1 or more, 2 or more, 3 or more, 4 or more, 5 or more, or 6 or more. In some embodiments, the NAS for a sample from the subject following administration is reduced by 1, 2, 3, 4, 5, or 6. In some embodiments, the NAFLD activity score (NAS) for a sample from the subject following administration during the period of time is reduced by 1 or more, 2 or more, 3 or more, 4 or more, 5 or more, or 6 or more. In some embodiments, the NAS for a sample from the subject following administration during the period of time is reduced by 1, 2, 3, 4, 5, or 6. In some embodiments, the sample from the subject is from a liver biopsy.

In some embodiments, the treatment of NAFLD, e.g., NAFL or NASH, comprises treatment of hepatic inflammation. In some embodiments, the severity of the hepatic inflammation is decreased by about 1% to about 50%, about 25% to about 75%, or about 50% to about 100%. In some embodiments, the severity of hepatic inflammation is decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, the treatment of NAFLD, e.g., NAFL or NASH, comprises treatment of fibrosis. In some embodiments, the treatment of the NAFLD comprises treatment of cirrhosis (e.g., stage 4 of fibrosis). In some embodiments, treatment of fibrosis comprises a decrease in the stage of fibrosis, for example, from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.

In some embodiments, the adiponectin level in the subject is increased by at least about 30%, at least about 68%, at least about 175%, or at least about 200%. In some embodiments, the increase is by at least about 175%.

In some embodiments, the level of aspartate aminotransferase (AST) in the subject does not increase. In some embodiments, the level of aspartate aminotransferase (AST) in the subject decreases. In some embodiments, the level of alanine aminotransferase (ALT) in the subject does not increase. In some embodiments, the level of alanine aminotransferase (ALT) in the subject decreases. In some embodiments, the total body weight of the subject does not increase. In some embodiments, the total body weight of the subject decreases. In some embodiments, the body mass index (BMI) of the subject does not increase. In some embodiments, the body mass index (BMI) of the subject decreases. In some embodiments, the waist and hip (WTH) ratio of the subject does not increase. In some embodiments, the waist and hip (WTH) ratio of the subject decreases.

In some embodiments, a non-invasive liver fibrosis marker does not increase or decreases. In some embodiments, the non-invasive liver fibrosis marker is Enhanced Liver Fibrosis (ELF) panel.

In some embodiments, treatment of NAFLD comprises a decrease in the level of one or more biomarkers indicative of one or more of liver damage, inflammation, fibrosis, and/or cirrhosis, e.g., any of the biomarkers as described herein. In some embodiments, treatment of NAFLD comprises a decrease in the level of one or more biomarkers indicative of one or more of liver damage, inflammation, fibrosis, and/or cirrhosis by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

In some embodiments, the treatment of NAFLD decreases the level of serum bile acids in the subject. In some embodiments, the treatment of NAFLD comprises treatment of pruritus.

In some embodiments, the subject has liver fibrosis associated with the NAFLD. In some embodiments, the subject has hepatic cirrhosis (e.g., stage 4 fibrosis) associated with the NAFLD. In some embodiments, the subject has liver fibrosis as a comorbidity. In some embodiments, the subject has hepatic cirrhosis (e.g., stage 4 fibrosis) as a comorbidity. In some embodiments, the subject has liver fibrosis caused by the NAFLD. In some embodiments, the subject has hepatic cirrhosis (e.g., stage 4 fibrosis) caused by the NAFLD.

In some embodiments, the NAFLD is simple nonalcoholic fatty liver (NAFL). In some embodiments, the NAFLD is NAFL with attendant liver fibrosis. In some embodiments, the NAFLD is NAFL with attendant liver cirrhosis.

In some embodiments, the NAFLD is nonalcoholic steatohepatitis (NASH). In some embodiments, the NAFLD is NASH with attendant liver fibrosis. In some embodiments, the NAFLD is NASH with attendant liver cirrhosis.

In some embodiments, the method further comprises performing a liver biopsy to determine the NAFLD activity score of the biopsy sample obtained from the subject.

In some embodiments, the compound of formula (I), or a pharmaceutically acceptable salt or solvate thereof and: the SGLT-2 inhibitor and/or the GLP-1 receptor agonist, and the metformin are administered prophylactically. In some embodiments, the compound of formula (I), or a pharmaceutically acceptable salt or solvate thereof, the SGLT-2 inhibitor, the GLP-1 receptor agonist, and the metformin are administered prophylactically. In other embodiments, the compound of formula (I), or a pharmaceutically acceptable salt or solvate thereof and the SGLT-2 inhibitor, are administered prophylactically. In still other embodiments, the compound of formula (I), or a pharmaceutically acceptable salt or solvate thereof and the GLP-1 receptor agonist are administered prophylactically. In some other embodiments, the compound of formula (I), or a pharmaceutically acceptable salt or solvate thereof, the SGLT-2 inhibitor, and the metformin are administered prophylactically. In some embodiments, the compound of formula (I), or a pharmaceutically acceptable salt or solvate thereof, the GLP-1 receptor agonist, and the metformin are administered prophylactically.

In some embodiments, the subject was previously treated, before the period of time, with one or more therapeutic agents, e.g., treatment with at least one NAFLD treatment, NASH treatment, type 2 diabetes treatment, obesity treatment, metabolic syndrome treatment, liver disease treatment, cardiovascular treatment, heart failure treatment, hypertension treatment. In some embodiments, the one or more therapeutic agents that were administered to the patient before the period of time was unsuccessful (e.g., therapeutically unsuccessful as determined by a physician). In some embodiments, the unsuccessful treatment did not comprise or consist essentially of administration of the compound of formula (I), or a pharmaceutically acceptable salt or solvate thereof and: the SGLT-2 inhibitor and/or the GLP-1 receptor agonist. In other embodiments, the unsuccessful treatment did not comprise or consist essentially of administration of the compound of formula (I), or a pharmaceutically acceptable salt or solvate thereof and the SGLT-2 inhibitor. In still other embodiments, the unsuccessful treatment did not comprise or consist essentially of administration of the compound of formula (I), or a pharmaceutically acceptable salt or solvate thereof and the GLP-1 receptor agonist.

In some other embodiments, the unsuccessful treatment did not comprise or consist essentially of administration of the compound of formula (I), or a pharmaceutically acceptable salt or solvate thereof, the SGLT-2 inhibitor and the GLP-1 receptor agonist. In other embodiments, the unsuccessful treatment did not comprise or consist essentially of administration of the compound of formula (I), or a pharmaceutically acceptable salt or solvate thereof, the SGLT-2 inhibitor, and the metformin. In still other embodiments, the unsuccessful treatment did not comprise or consist essentially of administration of the compound of formula (I), or a pharmaceutically acceptable salt or solvate thereof, the GLP-1 receptor agonist, and the metformin. In some other embodiments, the unsuccessful treatment did not comprise or consist essentially of administration of the compound of formula (I), or a pharmaceutically acceptable salt or solvate thereof, the SGLT-2 inhibitor, the GLP-1 receptor agonist, and the metformin.

In some embodiments, the method of treating non-alcoholic fatty liver disease (NAFLD) in a subject in need thereof comprises or consists essentially of administering to the subject (a) the compound of Formula (I), or a pharmaceutically acceptable salt or solvate thereof, and (b) empagliflozin, or a pharmaceutically acceptable salt or solvate thereof, wherein the amounts of (a) and (b) together are effective in treating NAFLD. In some embodiments, a method of treating non-alcoholic fatty liver disease (NAFLD) in a subject in need thereof comprises or consists essentially of administering to the subject (a) the compound of Formula (I), or a pharmaceutically acceptable salt or solvate thereof, and (b) empagliflozin, or a pharmaceutically acceptable salt or solvate thereof, during a period of time, wherein the amounts of (a) and (b) together are effective in treating NAFLD.

In some embodiments, the method of treating NAFLD in a subject in need thereof comprises or consists essentially of administering to the subject a therapeutically effective amount of (a) the compound of Formula (I), or a pharmaceutically acceptable salt or solvate thereof, and (b) empagliflozin, or a pharmaceutically acceptable salt or solvate thereof. In some embodiments, a method of treating NAFLD in a subject in need thereof comprises or consists essentially of administering to the subject a therapeutically effective amount of (a) the compound of Formula (I), or a pharmaceutically acceptable salt or solvate thereof, and (b) empagliflozin, or a pharmaceutically acceptable salt or solvate thereof, during a period of time.

In some embodiments, the method further comprises administering (c) a GLP-1 receptor agonist, or a pharmaceutically acceptable salt and/or solvate thereof. In some embodiments, the GLP-1 receptor agonist is administered during the period of time. In some embodiments, the GLP-1 receptor agonist is selected from the group consisting of: liraglutide, dulaglutide, exenatide, taspoglutide, lixisenatide, albiglutide, semaglutide, GLP-1, or a combination of two or more thereof. In some embodiments, the GLP-1 receptor agonist is dulaglutide. In other embodiments, the GLP-1 receptor agonist is exenatide. In still other embodiments, the GLP-1 receptor agonist is liraglutide. In some embodiments, the GLP-1 receptor agonist is lixisenatide. In other embodiments, the GLP-1 receptor agonist is semaglutide. In some embodiments, 0.25 mg to 2 mg of duraglutide is administered. In other embodiments, 1 microgram (mcg) to 10 mcg of exenatide is administered. In still other embodiments, 0.5 mg to 3 mg of liraglutide is administered. In some embodiments, 10 mcg to 30 mcg or lixisenatide is administered. In other embodiments, 0.25 mg to 2 mg of semaglutide is administered.

In some embodiments, the method further comprises administering (d) metformin, or a pharmaceutically acceptable salt and/or solvate thereof.

In some embodiments, the method further comprises administering (c) a GLP-1 receptor agonist, or a pharmaceutically acceptable salt and/or solvate thereof and (d) metformin, or a pharmaceutically acceptable salt thereof. In some embodiments, the GLP-1 receptor agonist is administered during the period of time. In some embodiments, the GLP-1 receptor agonist is selected from the group consisting of: liraglutide, dulaglutide, exenatide, taspoglutide, lixisenatide, albiglutide, semaglutide, GLP-1, or a combination of two or more thereof. In some embodiments, the GLP-1 receptor agonist is dulaglutide. In other embodiments, the GLP-1 receptor agonist is exenatide. In still other embodiments, the GLP-1 receptor agonist is liraglutide. In some embodiments, the GLP-1 receptor agonist is lixisenatide. In other embodiments, the GLP-1 receptor agonist is semaglutide. In some embodiments, 0.25 mg to 2 mg of duraglutide is administered. In other embodiments, 1 microgram (mcg) to 10 mcg of exenatide is administered. In still other embodiments, 0.5 mg to 3 mg of liraglutide is administered. In some embodiments, 10 mcg to 30 mcg or lixisenatide is administered. In other embodiments, 0.25 mg to 2 mg of semaglutide is administered.

Also provided herein are methods of treating fibrosis in a subject in need thereof comprising or consisting essentially of administering to the subject (a) the compound of Formula (I), or a pharmaceutically acceptable salt or solvate thereof, and (b) an SGLT-2 inhibitor, or a pharmaceutically acceptable salt or solvate thereof, wherein the amounts of (a) and (b) together are effective in treating fibrosis. In some embodiments, a method of treating fibrosis in a subject in need thereof comprises or consists essentially of administering to the subject (a) the compound of Formula (I), or a pharmaceutically acceptable salt or solvate thereof, and (b) an SGLT-2 inhibitor, or a pharmaceutically acceptable salt or solvate thereof, during a period of time, wherein the amounts of (a) and (b) together are effective in treating fibrosis.

Provided herein are methods of treating fibrosis in a subject in need thereof comprising or consisting essentially of administering to the subject (a) a therapeutically effective amount of the compound of Formula (I), or a pharmaceutically acceptable salt or solvate thereof, and (b) a therapeutically effective amount of an SGLT-2 inhibitor, or a pharmaceutically acceptable salt or solvate thereof. In some embodiments, a method of treating fibrosis in a subject in need thereof comprises or consists essentially of administering to the subject (a) a therapeutically effective amount of the compound of Formula (I), or a pharmaceutically acceptable salt or solvate thereof, and (b) a therapeutically effective amount of an SGLT-2 inhibitor, or a pharmaceutically acceptable salt or solvate thereof, during a period of time. In some embodiments, the amounts of (a) and (b) together are effective in treating fibrosis.

In some embodiments, the fibrosis is cirrhosis (e.g., stage 4 of fibrosis). In some embodiments, the fibrosis is associated with NAFLD (e.g., NAFL or NASH). In some embodiments, the cirrhosis is associated with the NAFLD (e.g., NAFL or NASH). In some embodiments, the fibrosis is caused by NAFLD (e.g., NAFL or NASH). In some embodiments, the cirrhosis is caused by the NAFLD (e.g., NAFL or NASH).

In some embodiments, the treatment of fibrosis comprises a decrease in the severity of the fibrosis, a lack of progression of the fibrosis, or a slowing of the progression of the fibrosis. In some embodiments, treatment of fibrosis comprises a decrease in the stage of fibrosis, for example, from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.

Also provided herein are methods of treating hepatic steatosis in a subject in need thereof comprising or consisting essentially of administering to the subject (a) the compound of Formula (I), or a pharmaceutically acceptable salt or solvate thereof, and (b) an SGLT-2 inhibitor, or a pharmaceutically acceptable salt or solvate thereof, wherein the amounts of (a) and (b) together are effective in treating hepatic steatosis. In some embodiments, a method of treating hepatic steatosis in a subject in need thereof comprises or consists essentially of administering to the subject (a) the compound of Formula (I), or a pharmaceutically acceptable salt or solvate thereof, and (b) an SGLT-2 inhibitor, or a pharmaceutically acceptable salt or solvate thereof, during a period of time, wherein the amounts of (a) and (b) together are effective in treating hepatic steatosis.

Provided herein are methods of treating hepatic steatosis in a subject in need thereof comprising or consisting essentially of administering to the subject (a) a therapeutically effective amount of the compound of Formula (I), or a pharmaceutically acceptable salt or solvate thereof, and (b) a therapeutically effective amount of an SGLT-2 inhibitor, or a pharmaceutically acceptable salt or solvate thereof. In some embodiments, a method of treating hepatic steatosis in a subject in need thereof comprises or consists essentially of administering to the subject (a) a therapeutically effective amount of the compound of Formula (I), or a pharmaceutically acceptable salt or solvate thereof, and (b) a therapeutically effective amount of an SGLT-2 inhibitor, or a pharmaceutically acceptable salt or solvate thereof, during a period of time. In some embodiments, wherein the amounts of (a) and (b) together are effective in treating hepatic steatosis.

Provided herein are methods of treating hepatic steatosis in a subject in need thereof comprising or consisting essentially of administering to the subject (a) a therapeutically effective amount of the compound of Formula (I), or a pharmaceutically acceptable salt or solvate thereof, and (b) a therapeutically effective amount of an SGLT-2 inhibitor, or a pharmaceutically acceptable salt or solvate thereof. In some embodiments, a method of treating hepatic steatosis in a subject in need thereof comprises or consists essentially of administering to the subject (a) a therapeutically effective amount of the compound of Formula (I), or a pharmaceutically acceptable salt or solvate thereof, and (b) a therapeutically effective amount of an SGLT-2 inhibitor, or a pharmaceutically acceptable salt or solvate thereof, during a period of time. In some embodiments, the amounts of (a) and (b) together are effective in treating hepatic steatosis.

In some embodiments, the treatment of hepatic steatosis comprises a reduction in the amount of hepatic steatosis by about 1% to about 50%, about 25% to about 75%, or about 50% to about 100%. In some embodiments, the treatment of hepatic steatosis comprises a reduction in the amount of hepatic steatosis by about 5%, bout 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, (a) and (b) are administered concurrently. In some embodiments, (a) and (b) are administered as a fixed combination. In some embodiments, (a) and (b) are administered as a non-fixed combination. In some embodiments, (a) and (b) are administered sequentially and in any order, at specific or varying time intervals (e.g., during the period of time). In some embodiments, a therapeutically effective amount of each of (a) and (b) are administered concurrently. In some embodiments, a therapeutically effective amount of each of (a) and (b) are administered sequentially and in any order, at specific or varying time intervals (e.g., during the period of time).

In some embodiments, the amount of the compound of Formula (I), or a pharmaceutically acceptable salt or solvate thereof, is from about 0.1 to about 15 mg. For example, from about 0.1 to about 10 mg, about 5 to about 15 mg, or about 2 to about 12 mg. In some embodiments, the compound of Formula (I), or a pharmaceutically acceptable salt or solvate thereof, is administered at a dose from about 0.1 to about 5 mg, about 0.1 to about 4 mg, about 0.5 to about 3 mg, about 0.5 to about 2 mg, about 0.5 to about 1 mg, about 1 to about 3 mg, about 2 to about 4 mg, about 3 to about 5 mg, about 1 to about 6 mg, about 2 to about 6 mg, about 3 to about 6 mg, about 4 to about 6 mg, or about 5 to about 6 mg. For example, about 0.10 mg, about 0.15 mg, about 0.20 mg, about 0.25 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1.00 mg, about 1.05 mg, about 1.10 mg, about 1.15 mg, about 1.20 mg, about 1.25 mg, about 1.30 mg, about 1.35 mg, about 1.40 mg, about 1.45 mg, about 1.50 mg, about 1.55 mg, about 1.60 mg, about 1.65 mg, about 1.70 mg, about 1.75 mg, about 1.80 mg, about 1.85 mg, about 1.90 mg, about 1.95 mg, about 2.00 mg, about 2.05 mg, about 2.10 mg, about 2.15 mg, about 2.20 mg, about 2.25 mg, about 2.30 mg, about 2.35 mg, about 2.40 mg, about 2.45 mg, about 2.50 mg, about 2.55 mg, about 2.60 mg, about 2.65 mg, about 2.70 mg, about 2.75 mg, about 2.80 mg, about 2.85 mg, about 2.90 mg, about 2.95 mg, about 3.00 mg, about 3.05 mg, about 3.10 mg, about 3.15 mg, about 3.20 mg, about 3.25 mg, about 3.30 mg, about 3.35 mg, about 3.40 mg, about 3.45 mg, about 3.50 mg, about 3.55 mg, about 3.60 mg, about 3.65 mg, about 3.70 mg, about 3.75 mg, about 3.80 mg, about 3.85 mg, about 3.90 mg, about 3.95 mg, about 4.00 mg, about 4.05 mg, about 4.10 mg, about 4.15 mg, about 4.20 mg, about 4.25 mg, about 4.30 mg, about 4.35 mg, about 4.40 mg, about 4.45 mg, about 4.50 mg, about 4.55 mg, about 4.60 mg, about 4.65 mg, about 4.70 mg, about 4.75 mg, about 4.80 mg, about 4.85 mg, about 4.90 mg, about 4.95 mg, about 5.00 mg, about 5.05 mg, about 5.10 mg, about 5.15 mg, about 5.20 mg, about 5.25 mg, about 5.30 mg, about 5.35 mg, about 5.40 mg, about 5.45 mg, about 5.50 mg, about 5.55 mg, about 5.60 mg, about 5.65 mg, about 5.70 mg, about 5.75 mg, about 5.80 mg, about 5.85 mg, about 5.90 mg, about 5.95 mg, about 6.00 mg, about 6.05 mg, about 6.10 mg, about 6.15 mg, about 6.20 mg, about 6.25 mg, about 6.30 mg, about 6.35 mg, about 6.40 mg, about 6.45 mg, about 6.50 mg, about 6.55 mg, about 6.60 mg, about 6.65 mg, about 6.70 mg, about 6.75 mg, about 6.80 mg, about 6.85 mg, about 6.90 mg, about 6.95 mg, about 7.00 mg, about 7.05 mg, about 7.10 mg, about 7.15 mg, about 7.20 mg, about 7.25 mg, about 7.30 mg, about 7.35 mg, about 7.40 mg, about 7.45 mg, about 7.50 mg, about 7.55 mg, about 7.60 mg, about 7.65 mg, about 7.70 mg, about 7.75 mg, about 7.80 mg, about 7.85 mg, about 7.90 mg, about 7.95 mg, about 8.00 mg, about 8.05 mg, about 8.10 mg, about 8.15 mg, about 8.20 mg, about 8.25 mg, about 8.30 mg, about 8.35 mg, about 8.40 mg, about 8.45 mg, about 8.50 mg, about 8.55 mg, about 8.60 mg, about 8.65 mg, about 8.70 mg, about 8.75 mg, about 8.80 mg, about 8.85 mg, about 8.90 mg, about 8.95 mg, about 9.00 mg, about 9.05 mg, about 9.10 mg, about 9.15 mg, about 9.20 mg, about 9.25 mg, about 9.30 mg, about 9.35 mg, about 9.40 mg, about 9.45 mg, about 9.50 mg, about 9.55 mg, about 9.60 mg, about 9.65 mg, about 9.70 mg, about 9.75 mg, about 9.80 mg, about 9.85 mg, about 9.90 mg, about 9.95 mg, or about 10.00 mg. In some embodiments, the dose is a therapeutically effective amount.

In some embodiments described herein, including, for example, methods, pharmaceutical compositions, and pharmaceutical combinations, the compound of Formula (I), or a pharmaceutically acceptable salt or solvate thereof, is administered to the subject daily and at a dose of about 3 mg. In some embodiments, the compound of Formula (I), or a pharmaceutically acceptable salt or solvate thereof, is administered at a dose from about 0.1 to about 10.0 mg per day. In some embodiments, the compound of Formula (I), or a pharmaceutically acceptable salt or solvate thereof, is administered at a dose from about 0.1 to about 3 mg per day. In some embodiments, the compound of Formula (I), or a pharmaceutically acceptable salt or solvate thereof, is administered at a dose about 0.5 milligram per day. In some embodiments, the compound of Formula (I), or a pharmaceutically acceptable salt or solvate thereof, is administered at a dose about 1 milligram per day. In some embodiments, the compound of Formula (I), or a pharmaceutically acceptable salt or solvate thereof, is administered at a dose about 2 mg per day.

In some embodiments, the compound of Formula (I), or a pharmaceutically acceptable salt or solvate thereof, is administered to the subject twice a day, daily, every other day, three times a week, twice a week, weekly, every other week, twice a month, or monthly. In some embodiments, the compound of Formula (I), or a pharmaceutically acceptable salt or solvate thereof, is administered to the subject daily.

In some embodiments, the SGLT-2 inhibitor is selected from the group consisting of: empagliflozin, canagliflozin, dapagliflozin (including dapagliflozin propylene glycol hydrate), ertugliflozin, ipragliflozin, luseogliflozin, remogliflozin etabonate, serfliflozin etabonate, sotagliflozin, tofogliflozin, or a combination of two or more thereof. In some embodiments, the SGLT-2 inhibitor is empagliflozin. In other embodiments, the SGLT-2 inhibitor is dapagliflozin propylene glycol hydrate.

In some embodiments, the amount of the SGLT-2 inhibitor, or a pharmaceutically acceptable salt or solvate thereof, is from about 1 to about 350 mg. For example, about 1 to about 175 mg, about 175 to about 350 mg, or about 90 to about 260 mg. In some embodiments, the amount of the SGLT-2 inhibitor, or a pharmaceutically acceptable salt or solvate thereof, is from about 85 to about 325 mg. In some embodiments, the amount of the SGLT-2 inhibitor, or a pharmaceutically acceptable salt or solvate thereof, is from about 1 to about 50 mg, about 20 to about 70 mg, about 50 to about 100 mg, about 70 to about 120 mg, about 90 to about 140 mg, about 110 to about 160 mg, about 130 to about 180 mg, about 150 to about 200 mg, about 170 to about 220 mg, about 190 to about 240 mg, about 210 to about 260 mg, about 230 to about 280 mg, about 250 to about 300 mg, about 270 to about 320 mg, or about 290 to about 350 mg. For example, about 100 mg or about 300 mg. In some embodiments, the amount of the SGLT-2 inhibitor, or a pharmaceutically acceptable salt or solvate thereof, is from about 1 to about 15 mg. For example, about 1 to about 10 mg or about 5 to about 15 mg. In some embodiments, the amount of the SGLT-2 inhibitor, or a pharmaceutically acceptable salt or solvate thereof, is from 1 to about 3 mg, about 2 to about 4 mg, about 3 to about 5 mg, about 4 to about 6 mg, about 5 to about 7, about 6 to about 8, about 7 to about 9 mg, about 8 to about 10 mg, about 9 to about 11 mg, about 10 to about 12 mg, about 11 to about 13 mg, about 12 to about 14 mg, or about 13 to about 15 mg. For example, about 0.10 mg, about 0.15 mg, about 0.20 mg, about 0.25 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1.00 mg, about 1.05 mg, about 1.10 mg, about 1.15 mg, about 1.20 mg, about 1.25 mg, about 1.30 mg, about 1.35 mg, about 1.40 mg, about 1.45 mg, about 1.50 mg, about 1.55 mg, about 1.60 mg, about 1.65 mg, about 1.70 mg, about 1.75 mg, about 1.80 mg, about 1.85 mg, about 1.90 mg, about 1.95 mg, about 2.00 mg, about 2.05 mg, about 2.10 mg, about 2.15 mg, about 2.20 mg, about 2.25 mg, about 2.30 mg, about 2.35 mg, about 2.40 mg, about 2.45 mg, about 2.50 mg, about 2.55 mg, about 2.60 mg, about 2.65 mg, about 2.70 mg, about 2.75 mg, about 2.80 mg, about 2.85 mg, about 2.90 mg, about 2.95 mg, about 3.00 mg, about 3.05 mg, about 3.10 mg, about 3.15 mg, about 3.20 mg, about 3.25 mg, about 3.30 mg, about 3.35 mg, about 3.40 mg, about 3.45 mg, about 3.50 mg, about 3.55 mg, about 3.60 mg, about 3.65 mg, about 3.70 mg, about 3.75 mg, about 3.80 mg, about 3.85 mg, about 3.90 mg, about 3.95 mg, about 4.00 mg, about 4.05 mg, about 4.10 mg, about 4.15 mg, about 4.20 mg, about 4.25 mg, about 4.30 mg, about 4.35 mg, about 4.40 mg, about 4.45 mg, about 4.50 mg, about 4.55 mg, about 4.60 mg, about 4.65 mg, about 4.70 mg, about 4.75 mg, about 4.80 mg, about 4.85 mg, about 4.90 mg, about 4.95 mg, about 5.00 mg, about 5.05 mg, about 5.10 mg, about 5.15 mg, about 5.20 mg, about 5.25 mg, about 5.30 mg, about 5.35 mg, about 5.40 mg, about 5.45 mg, about 5.50 mg, about 5.55 mg, about 5.60 mg, about 5.65 mg, about 5.70 mg, about 5.75 mg, about 5.80 mg, about 5.85 mg, about 5.90 mg, about 5.95 mg, about 6.00 mg, about 6.05 mg, about 6.10 mg, about 6.15 mg, about 6.20 mg, about 6.25 mg, about 6.30 mg, about 6.35 mg, about 6.40 mg, about 6.45 mg, about 6.50 mg, about 6.55 mg, about 6.60 mg, about 6.65 mg, about 6.70 mg, about 6.75 mg, about 6.80 mg, about 6.85 mg, about 6.90 mg, about 6.95 mg, about 7.00 mg, about 7.05 mg, about 7.10 mg, about 7.15 mg, about 7.20 mg, about 7.25 mg, about 7.30 mg, about 7.35 mg, about 7.40 mg, about 7.45 mg, about 7.50 mg, about 7.55 mg, about 7.60 mg, about 7.65 mg, about 7.70 mg, about 7.75 mg, about 7.80 mg, about 7.85 mg, about 7.90 mg, about 7.95 mg, about 8.00 mg, about 8.05 mg, about 8.10 mg, about 8.15 mg, about 8.20 mg, about 8.25 mg, about 8.30 mg, about 8.35 mg, about 8.40 mg, about 8.45 mg, about 8.50 mg, about 8.55 mg, about 8.60 mg, about 8.65 mg, about 8.70 mg, about 8.75 mg, about 8.80 mg, about 8.85 mg, about 8.90 mg, about 8.95 mg, about 9.00 mg, about 9.05 mg, about 9.10 mg, about 9.15 mg, about 9.20 mg, about 9.25 mg, about 9.30 mg, about 9.35 mg, about 9.40 mg, about 9.45 mg, about 9.50 mg, about 9.55 mg, about 9.60 mg, about 9.65 mg, about 9.70 mg, about 9.75 mg, about 9.80 mg, about 9.85 mg, about 9.90 mg, about 9.95 mg, about 10.00 mg, about 11 mg, about 12 mg, about 13 mg, or about 15 mg.

In some embodiments, the SGLT-2 inhibitor, or a pharmaceutically acceptable salt or solvate thereof, is administered to the subject twice a day, daily, every other day, three times a week, twice a week, weekly, every other week, twice a month, or monthly. In some embodiments, the SGLT-2 inhibitor, or a pharmaceutically acceptable salt or solvate thereof, is administered to the subject daily.

In some embodiments, the method further comprises administering (c) a GLP-1 receptor agonist. In some embodiments, the GLP-1 receptor agonist is administered during the period of time. In some embodiments, the GLP-1 receptor agonist is selected from the group consisting of: liraglutide, dulaglutide, exenatide, taspoglutide, lixisenatide, albiglutide, semaglutide, GLP-1, or a combination of two or more thereof. In some embodiments, the GLP-1 receptor agonist is liraglutide. In some embodiments, the GLP-1 receptor agonist is dulaglutide. In other embodiments, the GLP-1 receptor agonist is exenatide. In still other embodiments, the GLP-1 receptor agonist is liraglutide. In some embodiments, the GLP-1 receptor agonist is lixisenatide. In other embodiments, the GLP-1 receptor agonist is semaglutide. In some embodiments, 0.25 mg to 2 mg of duraglutide is administered. In other embodiments, 1 microgram (mcg) to 10 mcg of exenatide is administered. In still other embodiments, 0.5 mg to 3 mg of liraglutide is administered. In some embodiments, 10 mcg to 30 mcg or lixisenatide is administered. In other embodiments, 0.25 mg to 2 mg of semaglutide is administered.

In some embodiments, the GLP-1 receptor agonist, or a pharmaceutically acceptable salt or solvate thereof, is administered to the subject twice a day, daily, every other day, three times a week, twice a week, weekly, every other week, twice a month, or monthly. In some embodiments, the GLP-1 receptor agonist, or a pharmaceutically acceptable salt or solvate thereof, is administered to the subject daily.

In some embodiments, the method further comprises administering (d) metformin, or a pharmaceutically acceptable salt and/or solvate thereof.

In some embodiments, the method further comprises administering (c) a GLP-1 receptor agonist, or a pharmaceutically acceptable salt and/or solvate thereof and (d) metformin, or a pharmaceutically acceptable salt thereof.

Also provided herein are pharmaceutical compositions comprising or consisting essentially of (a) the compound of Formula (I), or a pharmaceutically acceptable salt or solvate thereof, (b) an SGLT-2 inhibitor, or a pharmaceutically acceptable salt or solvate thereof, and one or more pharmaceutical excipients, wherein the amounts of (a) and (b) together are effective in treating NAFLD.

Also provided herein are pharmaceutical compositions comprising or consisting essentially of (a) a therapeutically effective amount of the compound of Formula (I), or a pharmaceutically acceptable salt or solvate thereof, (b) a therapeutically effective amount of an SGLT-2 inhibitor, or a pharmaceutically acceptable salt or solvate thereof, and one or more pharmaceutical excipients.

In some embodiments, the method further comprises administering (d) metformin, or a pharmaceutically acceptable salt and/or solvate thereof.

In some embodiments, the method further comprises administering (c) a GLP-1 receptor agonist, or a pharmaceutically acceptable salt and/or solvate thereof and (d) metformin, or a pharmaceutically acceptable salt thereof.

Also provided herein are pharmaceutical combinations comprising or consisting essentially of (a) the compound of Formula (I), or a pharmaceutically acceptable salt or solvate thereof, and (b) an SGLT-2 inhibitor, or a pharmaceutically acceptable salt or solvate thereof, and one or more pharmaceutical excipients, for concurrent or sequential administration for use in the treatment of non-alcoholic fatty liver disease (NAFLD). In some embodiments, the pharmaceutical combination further comprises at least one pharmaceutically acceptable carrier.

Also provided herein are pharmaceutical combinations comprising or consisting essentially of (a) the compound of Formula (I), or a pharmaceutically acceptable salt or solvate thereof, and (b) an SGLT-2 inhibitor, or a pharmaceutically acceptable salt or solvate thereof, and one or more pharmaceutical excipients, for concurrent or sequential administration during a period of time for use in the treatment of non-alcoholic fatty liver disease (NAFLD). In some embodiments, the pharmaceutical combination further comprises at least one pharmaceutically acceptable carrier.

In some embodiments, (a) and (b) are administered concurrently. In some embodiments, (a) and (b) are administered as a fixed combination. In some embodiments, (a) and (b) are administered as a non-fixed combination. In some embodiments, (a) and (b) are administered sequentially and in any order, at specific or varying time intervals (e.g., during the period of time). In some embodiments, a therapeutically effective amount of each of (a) and (b) are administered concurrently. In some embodiments, a therapeutically effective amount of each of (a) and (b) are administered sequentially and in any order, at specific or varying time intervals (e.g., during the period of time).

In some embodiments, the pharmaceutical combination further comprises (c) a GLP-1 receptor agonist. In some embodiments, the GLP-1 receptor agonist is administered during the period of time. In some embodiments, the GLP-1 receptor agonist is selected from the group consisting of: liraglutide, dulaglutide, exenatide, taspoglutide, lixisenatide, albiglutide, semaglutide, GLP-1, or a combination of two or more thereof. In some embodiments, the GLP-1 receptor agonist is liraglutide. In some embodiments, the GLP-1 receptor agonist is dulaglutide. In other embodiments, the GLP-1 receptor agonist is exenatide. In still other embodiments, the GLP-1 receptor agonist is liraglutide. In some embodiments, the GLP-1 receptor agonist is lixisenatide. In other embodiments, the GLP-1 receptor agonist is semaglutide. In some embodiments, 0.25 mg to 2 mg of duraglutide is administered. In other embodiments, 1 microgram (mcg) to 10 mcg of exenatide is administered. In still other embodiments, 0.5 mg to 3 mg of liraglutide is administered. In some embodiments, 10 mcg to 30 mcg or lixisenatide is administered. In other embodiments, 0.25 mg to 2 mg of semaglutide is administered.

In some embodiments, the pharmaceutical combination further comprises administering (d) metformin, or a pharmaceutically acceptable salt and/or solvate thereof.

In some embodiments, the pharmaceutical combination further comprises administering (c) a GLP-1 receptor agonist, or a pharmaceutically acceptable salt and/or solvate thereof and (d) metformin, or a pharmaceutically acceptable salt thereof.

Also provided herein are methods of treating non-alcoholic fatty liver disease (NAFLD) in a subject in need thereof comprising or consisting essentially of administering to the subject (a) the compound of Formula (I), or a pharmaceutically acceptable salt or solvate thereof, and (b) a GLP-1 receptor agonist, or a pharmaceutically acceptable salt or solvate thereof, wherein the amounts of (a) and (b) together are effective in treating NAFLD. In some embodiments, a method of treating non-alcoholic fatty liver disease (NAFLD) in a subject in need thereof comprises or consists essentially of administering to the subject (a) the compound of Formula (I), or a pharmaceutically acceptable salt or solvate thereof, and (b) a GLP-1 receptor agonist, or a pharmaceutically acceptable salt or solvate thereof, during a period of time, wherein the amounts of (a) and (b) together are effective in treating NAFLD.

Also provided herein are methods of treating a subject, the method comprising: selecting a subject having non-alcoholic fatty liver disease (NAFLD); and administering (a) the compound of Formula (I), or a pharmaceutically acceptable salt or solvate thereof, and (b) a GLP-1 receptor agonist, or a pharmaceutically acceptable salt or solvate thereof, to the selected subject wherein the amounts of (a) and (b) together are effective in treating NAFLD. In some embodiments, (a) and (b) are administered during a period of time.

Also provided here are methods of treating a subject, the method comprising: identifying a subject having non-alcoholic fatty liver disease (NAFLD); and administering (a) the compound of Formula (I), or a pharmaceutically acceptable salt or solvate thereof, and (b) a GLP-1 receptor agonist, or a pharmaceutically acceptable salt or solvate thereof, to the selected subject wherein the amounts of (a) and (b) together are effective in treating NAFLD. In some embodiments, (a) and (b) are administered during a period of time.

Also provided herein are methods of treating NAFLD in a subject in need thereof comprising or consisting essentially of administering to the subject (a) a therapeutically effective amount of the compound of Formula (I), or a pharmaceutically acceptable salt or solvate thereof, and (b) a therapeutically effective amount of a GLP-1 receptor agonist, or a pharmaceutically acceptable salt or solvate thereof. In some embodiments, a method of treating NAFLD in a subject in need thereof comprises or consists essentially of administering to the subject (a) a therapeutically effective amount of the compound of Formula (I), or a pharmaceutically acceptable salt or solvate thereof, and (b) a therapeutically effective amount of a GLP-1 receptor agonist, or a pharmaceutically acceptable salt or solvate thereof, during a period of time. In some embodiments, the amounts of (a) and (b) together are effective in treating NAFLD.

Also provided herein are methods of treating a subject, the method comprising: selecting a subject having non-alcoholic fatty liver disease (NAFLD); and administering (a) a therapeutically effective amount of the compound of Formula (I), or a pharmaceutically acceptable salt or solvate thereof, and (b) a therapeutically effective amount of a GLP-1 receptor agonist, or a pharmaceutically acceptable salt or solvate thereof, to the selected subject. In some embodiments, (a) and (b) are administered during a period of time. In some embodiments, the amounts of (a) and (b) together are effective in treating NAFLD.

Also provided here are methods of selecting a subject for treatment, the method comprising: identifying a subject having NAFLD; and selecting the identified subject for treatment with a (a) therapeutically effective amount of the compound of Formula (I), or a pharmaceutically acceptable salt or solvate thereof, and (b) a therapeutically effective amount of a GLP-1 receptor agonist, or a pharmaceutically acceptable salt or solvate thereof. In some embodiments, the amounts of (a) and (b) together are effective in treating NAFLD.

Also provided herein are methods of selecting a subject for participation in a clinical trial, the method comprising: identifying a subject having NAFLD; and selecting the identified subject for participation in a clinical trial that comprises administration of (a) a therapeutically effective amount of the compound of Formula (I), or a pharmaceutically acceptable salt or solvate thereof, and (b) a therapeutically effective amount of a GLP-1 receptor agonist, or a pharmaceutically acceptable salt or solvate thereof. In some embodiments, the amounts of (a) and (b) together are effective in treating NAFLD.

In some embodiments, (a) and (b) are administered concurrently. In some embodiments, (a) and (b) are administered as a fixed combination. In some embodiments, (a) and (b) are administered as a non-fixed combination. In some embodiments, (a) and (b) are administered sequentially and in any order, at specific or varying time intervals (e.g., during the period of time). In some embodiments, a therapeutically effective amount of each of (a) and (b) are administered concurrently. In some embodiments, a therapeutically effective amount of each of (a) and (b) are administered sequentially and in any order, at specific or varying time intervals (e.g., during the period of time).

In some embodiments, the GLP-1 receptor agonist is selected from the group consisting of: liraglutide, dulaglutide, exenatide, taspoglutide, lixisenatide, albiglutide, semaglutide, GLP-1, or a combination thereof. In some embodiments, the GLP-1 receptor agonist is liraglutide.

In some embodiments, treatment of NAFLD comprises a decrease of one or more symptoms associated with NAFLD in the subject. Exemplary symptoms can include one or more of an enlarged liver, fatigue, pain in the upper right abdomen, abdominal swelling, enlarged blood vessels just beneath the skin's surface, enlarged breasts in men, enlarged spleen, red palms, jaundice, and pruritus. In some embodiments, the subject is asymptomatic.

In some embodiments, the treatment of NAFLD, e.g., NAFL or NASH, comprises a reduction in hepatic steatosis. For example, hepatic steatosis is decreased by at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more than 99% following administration of (a) and (b) for a period of time.

In some embodiments, the treatment of NAFLD, e.g., NAFL or NASH, is assessed using the NAFLD Activity Score (NAS). In some embodiments, treatment of NAFLD comprises a decrease in the NAS. In some embodiments, the NAS for a sample from the subject following administration is 7 or less. In some embodiments, the NAS for a sample from the subject following administration is 5 or less, 4 or less, 3 or less, or 2 or less. In some embodiments, the NAFLD activity score (NAS) for a sample from the subject following administration during the period of time is 7 or less. In some embodiments, the NAS for a sample from the subject following administration during the period of time is 5 or less, 4 or less, 3 or less, or 2 or less. In some embodiments, the sample from the subject is from a liver biopsy.

In some embodiments, the treatment of NAFLD, e.g., NAFL or NASH, can be assessed using the NAFLD Activity Score (NAS). In some embodiments, the NAS for a sample from the subject following administration is reduced by 1 or more, 2 or more, 3 or more, 4 or more, 5 or more, or 6 or more. In some embodiments, the NAS for a sample from the subject following administration is reduced by 1, 2, 3, 4, 5, or 6. In some embodiments, the NAFLD activity score (NAS) for a sample from the subject following administration during the period of time is reduced by 1 or more, 2 or more, 3 or more, 4 or more, 5 or more, or 6 or more. In some embodiments, the NAS for a sample from the subject following administration during the period of time is reduced by 1, 2, 3, 4, 5, or 6. In some embodiments, the sample from the subject is from a liver biopsy.

In some embodiments, the treatment of NAFLD, e.g., NAFL or NASH, comprises treatment of hepatic inflammation. In some embodiments, the severity of the hepatic inflammation is decreased by about 1% to about 50%, about 25% to about 75%, or about 50% to about 100%. In some embodiments, the severity of hepatic inflammation is decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, the treatment of NAFLD, e.g., NAFL or NASH, comprises treatment of fibrosis. In some embodiments, the treatment of the NAFLD comprises treatment of cirrhosis (e.g., stage 4 of fibrosis). In some embodiments, treatment of fibrosis comprises a decrease in the stage of fibrosis, for example, from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.

In some embodiments, the adiponectin level in the subject is increased by at least about 30%, at least about 68%, at least about 175%, or at least about 200%. In some embodiments, the increase is by at least about 175%.

In some embodiments, the level of aspartate aminotransferase (AST) in the subject does not increase. In some embodiments, the level of aspartate aminotransferase (AST) in the subject decreases. In some embodiments, the level of alanine aminotransferase (ALT) in the subject does not increase. In some embodiments, the level of alanine aminotransferase (ALT) in the subject decreases. In some embodiments, the total body weight of the subject does not increase. In some embodiments, the total body weight of the subject decreases. In some embodiments, the body mass index (BMI) of the subject does not increase. In some embodiments, the body mass index (BMI) of the subject decreases. In some embodiments, the waist and hip (WTH) ratio of the subject does not increase. In some embodiments, the waist and hip (WTH) ratio of the subject decreases.

In some embodiments, treatment of NAFLD comprises a decrease in the level of one or more biomarkers indicative of one or more of liver damage, inflammation, fibrosis, and/or cirrhosis, e.g., any of the biomarkers as described herein. In some embodiments, treatment of NAFLD comprises a decrease in the level of one or more biomarkers indicative of one or more of liver damage, inflammation, fibrosis, and/or cirrhosis by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

In some embodiments, the treatment of NAFLD decreases the level of serum bile acids in the subject. In some embodiments, the treatment of NAFLD comprises treatment of pruritus.

In some embodiments, the subject has liver fibrosis associated with the NAFLD. In some embodiments, the subject has hepatic cirrhosis (e.g., stage 4 fibrosis) associated with the NAFLD. In some embodiments, the subject has liver fibrosis as a comorbidity. In some embodiments, the subject has hepatic cirrhosis (e.g., stage 4 fibrosis) as a comorbidity. In some embodiments, the subject has liver fibrosis caused by the NAFLD. In some embodiments, the subject has hepatic cirrhosis (e.g., stage 4 fibrosis) caused by the NAFLD.

In some embodiments, the NAFLD is simple nonalcoholic fatty liver (NAFL). In some embodiments, the NAFLD is NAFL with attendant liver fibrosis. In some embodiments, the NAFLD is NAFL with attendant liver cirrhosis.

In some embodiments, the NAFLD is nonalcoholic steatohepatitis (NASH). In some embodiments, the NAFLD is NASH with attendant liver fibrosis. In some embodiments, the NAFLD is NASH with attendant liver cirrhosis.

In some embodiments, the method further comprises performing a liver biopsy to determine the NAFLD activity score of the biopsy sample obtained from the subject. In some embodiments, (a) and (b) are administered prophylactically.

In some embodiments, the subject was previously treated, before the period of time, with one or more therapeutic agents, e.g., treatment with at least one NAFLD treatment. In some embodiments, the one or more therapeutic agents that were administered to the patient before the period of time was unsuccessful (e.g., therapeutically unsuccessful as determined by a physician). In some embodiments, the unsuccessful treatment did not comprises or consist essentially of administration of (a) and (b).

In some embodiments, the subject has Type I diabetes as a comorbidity. In other embodiments, the subject has Type II diabetes as a comorbidity. In some embodiments, the subject has adequate glycemic control, prior to receiving the combination of (a) and (b). For example, in some embodiments, the subject has an HbA_{1c} level of ≤10%, or ≤9%, or ≤8%, or ≤7%, or ≤6%, or ≤5%, or ≤4%, or any value in between, prior to receiving the combination of (a) and (b). In some embodiments, the subject has an HbA_{1c} level of about 4% to about 6%, prior to receiving the combination of (a) and (b). In other embodiments, the subject has an HbA_{1c} level of about 5% to about 8%, prior to receiving the combination of (a) and (b). In still other embodiments, the subject has an HbA_{1c} level of about 6% to about 10%, prior to receiving the combination of (a) and (b). In some embodiments, the subject's HbA_{1c} level decreases by about 1% to about 5% after receiving the combination of (a) and (b); for example, about 1% to about 2%, about 1.5% to about 2.5%, about 2% to about 3%, about 2.5% to about 3.5%, about 3% to about 4%, about 3.5% to about 4.5%, about 4% to about 5%, or about 1.5% to about 3%, or any value in between. In some embodiments, the subject's HbA_{1c} level decreases by about 1.5% to about 3% after receiving the combination of (a) and (b). In some embodiments, the subject does not have Type I diabetes as a comorbidity. In other embodiments, the subject does not have Type II diabetes as a comorbidity.

In some embodiments, the subject has a mean fasting plasma glucose level of ≤170 mg/dL, ≤160 mg/dL, ≤150 mg/dL, ≤140 mg/dL, ≤130 mg/dL, ≤120 mg/dL, ≤110 mg/dL, or ≤100 mg/dL. In some embodiments, the subject has a mean fasting plasma glucose level, prior to receiving the combination of (a) and (b), of about 90 mg/dL to about 110 mg/dL. In other embodiments, the subject has a mean fasting plasma glucose level, prior to receiving the combination of (a) and (b), of about 100 mg/dL to about 120 mg/dL. In still other embodiments, the subject has a mean fasting plasma glucose level, prior to receiving the combination of (a) and (b), of about 110 mg/dL to about 130 mg/dL. In some other embodiments, the subject has a mean fasting plasma glucose level, prior to receiving the combination of (a) and (b), of about 120 mg/dL to about 140 mg/dL. In some embodiments, the subject has a mean fasting plasma glucose level, prior to receiving the combination of (a) and (b), of about 130 mg/dL to about 150 mg/dL. In other embodiments, the subject has a mean fasting plasma glucose level, prior to receiving the combination of (a) and (b), of about 140 mg/dL to about 160 mg/dL. In still other embodiments, the subject has a mean fasting plasma glucose level, prior to receiving the combination of (a) and (b), of about 150 mg/dL to about 170 mg/dL. In some embodiments, the subject's mean fasting plasma glucose level decreases by about 30 mg/dL to about 90 mg/dL after receiving the combination of (a) and (b); for example, by about 30 mg/dL to about 40 mg/dL, about 40 mg/dL to about 50 mg/dL, about 50 mg/dL to about 60 mg/dL, about 60 mg/dL to about 70 mg/dL, about 70 mg/dL to about 80 mg/dL, or about 80 mg/dL to about 90 mg/dL, or any value in between.

In some embodiments, the subject has a BMI of ≤35, ≤34, ≤33, ≤32, ≤31, ≤30, ≤29, ≤28, ≤27, ≤26, ≤25, ≤24, ≤23, ≤22, ≤21, or ≤20, or any value in between, prior to receiving the combination of (a) and (b). In some embodiments, the subject has a BMI of about 35 to about 40, prior to receiving the combination of (a) and (b). In other embodiments, the subject has a BMI of about 32 to about 35, prior to receiving the combination of (a) and (b). In still other embodiments, the subject has a BMI of about 28 to about 32, prior to receiving the combination of (a) and (b). In some other embodiments, the subject has a BMI of about 26 to about 30, prior to receiving the combination of (a) and (b). In yet other embodiments, the subject has a BMI of about 24 to about 28, prior to receiving the combination of (a) and (b). In some embodiments, the subject has a BMI of about 22 to about 26, prior to receiving the combination of (a) and (b). In other embodiments, the subject has a BMI of about 20 to about 24, prior to receiving the combination of (a) and (b). In some embodiments, the subject's BMI changes from about −10% to about +10% after receiving the combination of (a) and (b). In some embodiments, the subject's BMI decreases by about 0% to about 10% after receiving the combination of (a) and (b). In some embodiments, the subject's BMI decreases by about 0.5% to about 5% after receiving the combination of (a) and (b). In some embodiments, the decrease in the subject's BMI occurs within about 4 weeks to about 104 weeks; for example, about 4 weeks to about 8 weeks, about 6 weeks to about 12 weeks, about 8 weeks to about 16 weeks, about 12 weeks to about 24 weeks, about 16 weeks to about 40 weeks, about 24 weeks to about 52 weeks, about 32 weeks to about 64 weeks, about 40 weeks to about 80 weeks, about 52 weeks to about 96 weeks, about 72 weeks to about 104 weeks, or any value in between.

In some embodiments, the subject's weight changes from about −10% to about +10% after receiving the combination of (a) and (b). In some embodiments, the subject's weight changes from about −5% to about +5% after receiving the combination of (a) and (b). In some embodiments, the subject's weight decreases by about 0% to about 10% after receiving the combination of (a) and (b). In some embodiments, the subject's weight decreases by about 0.5% to about 5% after receiving the combination of (a) and (b). In some embodiments, the subject's weight changes from about −5 kg to about +5 kg after receiving the combination of (a) and (b). In some embodiments, the subject's weight changes from about −2 kg to about +2 kg after receiving the combination of (a) and (b). In some embodiments, the subject's weight decreases by about 0 kg to about 5 kg after receiving the combination of (a) and (b). In some embodiments, the subject's weight decreases by about 0.5 kg to about 2 kg after receiving the combination of (a) and (b). In some embodiments, the changes in the subject's weight occurs within about 4 weeks to about 104 weeks; for example, about 4 weeks to about 8 weeks, about 6 weeks to about 12 weeks, about 8 weeks to about 16 weeks, about 12 weeks to about 24 weeks, about 16 weeks to about 40 weeks, about 24 weeks to about 52 weeks, about 32 weeks to about 64 weeks, about 40 weeks to about 80 weeks, about 52 weeks to about 96 weeks, about 72 weeks to about 104 weeks, or any value in between.

In some embodiments, the method of treating non-alcoholic fatty liver disease (NAFLD) in a subject in need thereof comprises or consists essentially of administering to the subject (a) the compound of Formula (I), or a pharmaceutically acceptable salt or solvate thereof, and (b) liraglutide, or a pharmaceutically acceptable salt or solvate thereof, wherein the amounts of (a) and (b) together are effective in treating NAFLD. In some embodiments, a method of treating non-alcoholic fatty liver disease (NAFLD) in a subject in need thereof comprises or consists essentially of administering to the subject (a) the compound of Formula (I), or a pharmaceutically acceptable salt or solvate thereof, and (b) liraglutide, or a pharmaceutically acceptable salt or solvate thereof, during a period of time, wherein the amounts of (a) and (b) together are effective in treating NAFLD.

In some embodiments, the method of treating NAFLD in a subject in need thereof comprises or consists essentially of administering to the subject a therapeutically effective amount of (a) the compound of Formula (I), or a pharmaceutically acceptable salt or solvate thereof, and (b) liraglutide, or a pharmaceutically acceptable salt or solvate thereof. In some embodiments, a method of treating NAFLD in a subject in need thereof comprises or consists essentially of administering to the subject a therapeutically effective amount of (a) the compound of Formula (I), or a pharmaceutically acceptable salt or solvate thereof, and (b) liraglutide, or a pharmaceutically acceptable salt or solvate thereof, during a period of time.

In some embodiments, the method further comprises administering (c) an SGLT-2 inhibitor. In some embodiments, the SGLT-2 inhibitor is administered during the period of time. In some embodiments, the SGLT-2 inhibitor is selected from the group consisting of: empagliflozin, canagliflozin, dapagliflozin (including dapagliflozin propylene glycol hydrate), ertugliflozin, ipragliflozin, luseogliflozin, remogliflozin etabonate, serfliflozin etabonate, sotagliflozin, tofogliflozin, or a combination of two or more thereof. In some embodiments, the SGLT-2 inhibitor is empagliflozin. In other embodiments, the SGLT-2 inhibitor is dapagliflozin propylene glycol hydrate.

In some embodiments, the pharmaceutical combination further comprises administering (d) metformin, or a pharmaceutically acceptable salt and/or solvate thereof.

In some embodiments, the pharmaceutical combination further comprises administering (c) an SGLT-2 inhibitor, or a pharmaceutically acceptable salt and/or solvate thereof and (d) metformin, or a pharmaceutically acceptable salt thereof.

Also provided herein are methods of treating fibrosis in a subject in need thereof comprising or consisting essentially of administering to the subject (a) the compound of Formula (I), or a pharmaceutically acceptable salt or solvate thereof, and (b) a GLP-1 receptor agonist, or a pharmaceutically acceptable salt or solvate thereof, wherein the amounts of (a) and (b) together are effective in treating fibrosis. In some embodiments, a method of treating fibrosis in a subject in need thereof comprises or consists essentially of administering to the subject (a) the compound of Formula (I), or a pharmaceutically acceptable salt or solvate thereof, and (b) a GLP-1 receptor agonist, or a pharmaceutically acceptable salt or solvate thereof, during a period of time, wherein the amounts of (a) and (b) together are effective in treating fibrosis. In some embodiments, the fibrosis is cirrhosis (e.g., stage 4 of fibrosis).

Provided herein are methods of treating fibrosis in a subject in need thereof comprising or consisting essentially of administering to the subject (a) a therapeutically effective amount of the compound of Formula (I), or a pharmaceutically acceptable salt or solvate thereof, and (b) a therapeutically effective amount of a GLP-1 receptor agonist, or a pharmaceutically acceptable salt or solvate thereof. In some embodiments, a method of treating fibrosis in a subject in need thereof comprises or consists essentially of administering to the subject (a) a therapeutically effective amount of the compound of Formula (I), or a pharmaceutically acceptable salt or solvate thereof, and (b) a therapeutically effective amount of a GLP-1 receptor agonist, or a pharmaceutically acceptable salt or solvate thereof, during a period of time. In some embodiments, the amounts of (a) and (b) together are effective in treating fibrosis.

In some embodiments, the fibrosis is cirrhosis (e.g., stage 4 of fibrosis). In some embodiments, the fibrosis is associated with NAFLD (e.g., NAFL or NASH). In some embodiments, the cirrhosis is associated with the NAFLD (e.g., NAFL or NASH). In some embodiments, the fibrosis is caused by NAFLD (e.g., NAFL or NASH). In some embodiments, the cirrhosis is caused by the NAFLD (e.g., NAFL or NASH).

In some embodiments, the treatment of fibrosis comprises a decrease in the severity of the fibrosis, a lack of progression of the fibrosis, or a slowing of the progression of the fibrosis. In some embodiments, treatment of fibrosis comprises a decrease in the stage of fibrosis, for example, from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.

Also provided herein are methods of treating hepatic steatosis in a subject in need thereof comprising or consisting essentially of administering to the subject (a) the compound of Formula (I), or a pharmaceutically acceptable salt or solvate thereof, and (b) a GLP-1 receptor agonist, or a pharmaceutically acceptable salt or solvate thereof, wherein the amounts of (a) and (b) together are effective in treating hepatic steatosis. In some embodiments, a method of treating hepatic steatosis in a subject in need thereof comprises or consists essentially of administering to the subject (a) the compound of Formula (I), or a pharmaceutically acceptable salt or solvate thereof, and (b) a GLP-1 receptor agonist, or a pharmaceutically acceptable salt or solvate thereof, during a period of time, wherein the amounts of (a) and (b) together are effective in treating hepatic steatosis.

Provided herein are methods of treating hepatic steatosis in a subject in need thereof comprising or consisting essentially of administering to the subject (a) a therapeutically effective amount of the compound of Formula (I), or a pharmaceutically acceptable salt or solvate thereof, and (b) a therapeutically effective amount of a GLP-1 receptor agonist, or a pharmaceutically acceptable salt or solvate thereof. In some embodiments, a method of treating hepatic steatosis in a subject in need thereof comprises or consists essentially of administering to the subject (a) a therapeutically effective amount of the compound of Formula (I), or a pharmaceutically acceptable salt or solvate thereof, and (b) a therapeutically effective amount of a GLP-1 receptor agonist, or a pharmaceutically acceptable salt or solvate thereof, during a period of time. In some embodiments, the amounts of (a) and (b) together are effective in treating hepatic steatosis.

In some embodiments, the treatment of hepatic steatosis comprises a reduction in the amount of hepatic steatosis by about 1% to about 50%, about 25% to about 75%, or about 50% to about 100%. In some embodiments, the treatment of hepatic steatosis comprises a reduction in the amount of hepatic steatosis by about 5%, bout 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, (a) and (b) are administered concurrently. In some embodiments, (a) and (b) are administered as a fixed combination. In some embodiments, (a) and (b) are administered as a non-fixed combination. In some embodiments, (a) and (b) are administered sequentially and in any order, at specific or varying time intervals (e.g., during the period of time). In some embodiments, a therapeutically effective amount of each of (a) and (b) are administered concurrently. In some embodiments, a therapeutically effective amount of each of (a) and (b) are administered sequentially and in any order, at specific or varying time intervals (e.g., during the period of time).

In some embodiments, the GLP-1 receptor agonist is selected from the group consisting of: liraglutide, dulaglutide, exenatide, taspoglutide, lixisenatide, albiglutide, semaglutide, GLP-1, or a combination thereof. In some embodiments, the GLP-1 receptor agonist is liraglutide.

In some embodiments, the method further comprises administering (c) an SGLT-2 inhibitor. In some embodiments, the SGLT-2 inhibitor is administered during the period of time. In some embodiments, the SGLT-2 inhibitor is selected from the group consisting of: empagliflozin, canagliflozin, dapagliflozin (including dapagliflozin propylene glycol hydrate), ertugliflozin, ipragliflozin, luseogliflozin, remogliflozin etabonate, serfliflozin etabonate, sotagliflozin, tofogliflozin, or a combination of two or more thereof. In some embodiments, the SGLT-2 inhibitor is empagliflozin. In some embodiments, the SGLT-2 inhibitor is dapagliflozin propylene glycol hydrate.

In some embodiments, the pharmaceutical combination further comprises administering (d) metformin, or a pharmaceutically acceptable salt and/or solvate thereof.

In some embodiments, the pharmaceutical combination further comprises administering (c) an SGLT-2 inhibitor, or a pharmaceutically acceptable salt and/or solvate thereof and (d) metformin, or a pharmaceutically acceptable salt thereof.

Also provided herein are pharmaceutical compositions comprising or consisting essentially of (a) the compound of Formula (I), or a pharmaceutically acceptable salt or solvate thereof, (b) a GLP-1 receptor agonist, or a pharmaceutically acceptable salt or solvate thereof, and one or more pharmaceutical excipients, wherein the amounts of (a) and (b) together are effective in treating NAFLD.

Also provided herein are pharmaceutical compositions comprising or consisting essentially of (a) a therapeutically effective amount of the compound of Formula (I), or a pharmaceutically acceptable salt or solvate thereof, (b) a therapeutically effective amount of a GLP-1 receptor agonist, or a pharmaceutically acceptable salt or solvate thereof, and one or more pharmaceutical excipients.

In some embodiments, the GLP-1 receptor agonist is selected from the group consisting of: liraglutide, dulaglutide, exenatide, taspoglutide, lixisenatide, albiglutide, semaglutide, GLP-1, or a combination thereof. In some embodiments, the GLP-1 receptor agonist is liraglutide.

In some embodiments, the pharmaceutical composition further comprises (c) an SGLT-2 inhibitor. In some embodiments, the SGLT-2 inhibitor is selected from the group consisting of: empagliflozin, canagliflozin, dapagliflozin (including dapagliflozin propylene glycol hydrate), ertugliflozin, ipragliflozin, luseogliflozin, remogliflozin etabonate, serfliflozin etabonate, sotagliflozin, tofogliflozin, or a combination of two or more thereof. In some embodiments, the SGLT-2 inhibitor is empagliflozin. In other embodiments, the SGLT-2 inhibitor is dapagliflozin propylene glycol hydrate.

In some embodiments, the pharmaceutical composition further comprises administering (d) metformin, or a pharmaceutically acceptable salt and/or solvate thereof.

In some embodiments, the pharmaceutical composition further comprises administering (c) an SGLT-2 inhibitor, or a pharmaceutically acceptable salt and/or solvate thereof and (d) metformin, or a pharmaceutically acceptable salt thereof.

Also provided herein are pharmaceutical combinations comprising or consisting essentially of (a) the compound of Formula (I), or a pharmaceutically acceptable salt or solvate thereof, and (b) a GLP-1 receptor agonist, or a pharmaceutically acceptable salt or solvate thereof, and one or more pharmaceutical excipients, for concurrent or sequential administration for use in the treatment of non-alcoholic fatty liver disease (NAFLD). In some embodiments, the pharmaceutical combination further comprises at least one pharmaceutically acceptable carrier.

Also provided herein are pharmaceutical combinations comprising or consisting essentially of (a) the compound of Formula (I), or a pharmaceutically acceptable salt or solvate thereof, and (b) a GLP-1 receptor agonist, or a pharmaceutically acceptable salt or solvate thereof, and one or more pharmaceutical excipients, for concurrent or sequential administration during a period of time for use in the treatment of non-alcoholic fatty liver disease (NAFLD). In some embodiments, the pharmaceutical combination further comprises at least one pharmaceutically acceptable carrier.

In some embodiments, (a) and (b) are administered concurrently. In some embodiments, (a) and (b) are administered as a fixed combination. In some embodiments, (a) and (b) are administered as a non-fixed combination. In some embodiments, (a) and (b) are administered sequentially and in any order, at specific or varying time intervals (e.g., during the period of time). In some embodiments, a therapeutically effective amount of each of (a) and (b) are administered concurrently. In some embodiments, a therapeutically effective amount of each of (a) and (b) are administered sequentially and in any order, at specific or varying time intervals (e.g., during the period of time).

In some embodiments, the GLP-1 receptor agonist is selected from the group consisting of: liraglutide, dulaglutide, exenatide, taspoglutide, lixisenatide, albiglutide, semaglutide, GLP-1, or a combination thereof. In some embodiments, the GLP-1 receptor agonist is liraglutide.

In some embodiments, the pharmaceutical combination further comprises (c) an SGLT-2 inhibitor. In some embodiments, the SGLT-2 inhibitor is administered during the period of time. In some embodiments, the SGLT-2 inhibitor is selected from the group consisting of: empagliflozin, canagliflozin, dapagliflozin, ertugliflozin, ipragliflozin, luseogliflozin, remogliflozin etabonate, serfliflozin etabonate, sotagliflozin, tofogliflozin, or a combination of two or more thereof. In some embodiments, the SGLT-2 inhibitor is empagliflozin. In other embodiments, the SGLT-2 inhibitor is dapagliflozin propylene glycol hydrate.

In some embodiments, the pharmaceutical composition further comprises administering (d) metformin, or a pharmaceutically acceptable salt and/or solvate thereof.

In some embodiments, the pharmaceutical composition further comprises administering (c) an SGLT-2 inhibitor, or a pharmaceutically acceptable salt and/or solvate thereof and (d) metformin, or a pharmaceutically acceptable salt thereof.

### EXAMPLES

The following example further illustrates the invention. For example, the efficacy of CHS-131, alone or in combination with other therapeutic agents, to treat NAFLD is determined in the following examples.

### Example 1

This study assesses the effects of treatment with CHS-131 (Compound of Formula (I)), alone and in combination with other therapeutic agents, to treat NASH. Metabolic parameters, hepatic pathology, and NAFLD Activity Score including fibrosis stage are evaluated in male DIO-NASH mice.

In particular this study will permit mechanistic evaluation of the effects of CHS-131, an SGTL2-inhibitor (empagliflozin), or a GLP-1 inhibitor (liraglutide) monotherapy, and CH-131+an SGTL2-inhibitor (empagliflozin), and CH-131+a GLP-1 inhibitor (liraglutide) combination therapies on the NASH disease process. An overview of the study is provided in Table 3, below.

TABLE 3

| | | | | | Dosing | | | |
|---|---|---|---|---|---|---|---|---|
| | | Mouse | | Dose | | | | Time |
| Group | Compound | Model | n | (mg/kg) | Method | Volume | Frequency | of Day |
| 1 | Chow Vehicle + | LEAN- | 12 | NA | PO | 5 + 5 | QD | AM |
| | Vehicle | CHOW | | | | | | |
| 2 | NASH Vehicle + | DIO- | 12 | NA | PO | 5 + 5 | QD | AM |
| | Vehicle | NASH | to | | | | | |
| | | | 14 | | | | | |
| 3 | Low dose Compound | DIO- | 12 | 10 | PO | 5 + 5 | QD | AM |
| | of Formula (I) + | NASH | to | | | | | |
| | Vehicle | | 14 | | | | | |
| 4 | High dose Compound | DIO- | 12 | 30 | PO | 5 + 5 | QD | AM |
| | of Formula (I) + | NASH | to | | | | | |
| | Vehicle | | 14 | | | | | |
| 5 | Empagliflozin + | DIO- | 12 | 10 | PO | 5 + 5 | QD | AM |
| | Vehicle | NASH | to | | | | | |
| | | | 14 | | | | | |
| 6 | High dose Compound | DIO- | 12 | 30 + 10 | PO | 5 + 5 | QD | AM |
| | of Formula (I) + | NASH | to | | | | | |
| | Empagliflozin | | 14 | | | | | |
| 7 | Liraglutide + Vehicle | DIO- | 12 | 0.4 | SC + PO | 5 + 5 | QD | AM |
| | | NASH | to | | | | | |
| | | | 14 | | | | | |
| 8 | High dose Compound | DIO- | 12 | 30 + 0.4 | PO + SC | 5 + 5 | QD | AM |
| | of Formula (I) + | NASH | to | | | | | |
| | Liraglutide | | 14 | | | | | |
| 9 | Elafibranor + | DIO- | 12 | 30 | PO | 5 + 5 | QD | AM |
| | Vehicle | NASH | to | | | | | |
| | | | 14 | | | | | |

PO is per oral;
SC is subcutaneous;
QD is once a day. Groups 2-6 are fed a HF-HD diet.

Each animal is administered the respective compositions starting on Day 0 and ending on Day 82-84. The compositions are as described in Table 4.

TABLE 4
Compositions

| Name | Dissolved In | Process |
|---|---|---|
| Vehicle | 1% methyl cellulose in | N/A |
| | deionized water | |
| CHS-131 | 1% methyl cellulose in | Stir for 30-60 minutes before |
| | deionized water | and during dosing |
| Empagliflozin | 1% methyl cellulose in | Dissolved |
| | deionized water | |
| Liraglutide | PBS | Mix |
| Elafibranor | 1% methyl cellulose in | Mix |
| | deionized water | |

Samples, as described in Table 5, are collected before, during, and after the study.

TABLE 5
Samples collected over course of study

| Sample | Usage | Groups | Time Point | Method |
|---|---|---|---|---|
| Liver pre-biopsy | Stratification and | All | 3 weeks | Dissection |
| | randomization, NAFLD | | before start | |
| | Activity Score, Fibrosis | | of study | |
| | Stage, Col1a1 | | | |
| BG Baseline | Blood Glucose | All | 1 week | Tail Vein |
| | | | before start | |
| | | | of study | |
| Plasma insulin baseline | Plasma insulin | All | 1 week | Tail Vein |
| | | | before start | |
| | | | of study | |
| OGTT | Blood Glucose | All | Week 7-8 | Tail Vein |
| IPTT | Blood Glucose | All | Week 9/10 | Tail Vein |
| BG week 12 | Blood Glucose | All | Week 12 | Tail Vein |
| Plasma insulin week 12 | Plasma insulin | All | Week 12 | Tail Vein |
| Terminal | ALT/AST/TG/TC/BUN | All | Week 12 | Tail Vein |
| ALT/AST/TG/ | | | | |
| TC/BUN/creatinine | | | | |
| Liver post-biopsy | NAFLD Activity Score, | All | Termination | Dissection |
| | and fibrosis stage and | | | |
| | steatosis stage and Col1a1 | | | |
| | and Galectin-3 and a-SMA | | | |
| | quantification | | | |
| Liver TG/TC | Liver triglyceride and total | All | Termination | Dissection |
| | cholesterol | | | |
| Liver HP | Liver hydroxyproline | All | Termination | Dissection |
| Liver RNA | RNAseq (optional) | All | Termination | Dissection |
| Liver | Evaluation | All | Termination | Dissection |
| Muscle | Evaluation | All | Termination | Dissection |
| Epididymal fat | Evaluation | All | Termination | Dissection |
| Subcutaneous fat | Evaluation | All | Termination | Dissection |
| Kidney | Evaluation | All | Termination | Dissection |
| Brain | Evaluation | All | Termination | Dissection |
| Heart | Evaluation | All | Termination | Dissection |
| Terminal plasma | Evaluation | All | Termination | Cardiac |
| | | | | Puncture |

ALT is alanine transaminase;
a-SMA is alpha-smooth muscle actin;
AST is aspartate transaminase;
BG is blood glucose;
BUN is blood urea nitrogen;
Col1a1 is collagen 1a1;
OGTT is oral glucose tolerance test;
IPITT is intraperitoneal insulin tolerance test;
TG is triglycerides;
TC is total cholesterol;
HP is hydroxyproline

An overview of sample analyses that are performed during the study are listed in Tables 6-8, below.

TABLE 6
In vivo pharmacology

| Analysis | | | Period or | |
|---|---|---|---|---|
| Name | Groups | Samples | Frequency | Comments |
| Bodyweight | All | Whole | QD | na |
| | | animal | | |
| Food intake | All | Whole | QD week 0 + 1 | AM |
| | | animal | QW (24 h) Week 2-12 | |
| Echo MRI | All | Whole | Week 1 | na |
| baseline | | animal | | |
| Echo MRI | All | Whole | Week 11 | na |
| week 11 | | animal | | |
| Liver weight | All | Whole liver | Termination | na |
| | | weight (wet | | |
| | | weight) | | |

TABLE 7
Histology

| Analysis Name | Groups | Samples | Comments |
|---|---|---|---|
| Fibrosis stage | All | Liver pre-biopsy | PSR staining |
| | | Liver post-biopsy | Re-staining of pre-biopsy |
| NAFLD Activity | All | Liver pre-biopsy | HE staining |
| Score | | Liver post-biopsy | Re-staining of pre-biopsy |
| Col1a1 | All | Liver pre-biopsy | IHC (randomization) |
| | | Liver post-biopsy | IHC |
| Galectin-3 | All | Liver post-biopsy | IHC |
| quantification | | | |
| Steatosis | All | Liver post-biopsy | HE staining |
| quantification | | | |
| a-SMA | All | Liver post-biopsy | IHC |
| quantification | | | |

TABLE 8
Assays

| Analysis Name | Groups | Samples |
|---|---|---|
| Plasma insulin | All | Plasma insulin baseline |
| baseline | | |
| Plasma insulin | All | Plasma insulin baseline |
| week 12 | | |
| Plasma ALT | All | Terminal ALT/AST/TG/TC/BUN/Creatinine |
| Plasma AST | All | Terminal ALT/AST/TG/TC/BUN/Creatinine |
| Plasma TG | All | Terminal ALT/AST/TG/TC/BUN/Creatinine |
| Plasma TC | All | Terminal ALT/AST/TG/TC/BUN/Creatinine |
| Plasma BUN | All | Terminal ALT/AST/TG/TC/BUN/Creatinine |
| Plasma creatinine | All | Terminal ALT/AST/TG/TC/BUN/Creatinine |
| Liver | All | Liver TG/TC |
| triglycerides | | |
| Liver total | All | Liver TG/TC |
| cholesterol | | |
| Liver | All | Liver HP |
| hydroxyproline | | |

NAFLD Activity Score (NAS) and Fibrosis stage are evaluated as follows. Liver samples are fixed in formalin, paraffin embedded and sections are stained with hematoxylin and eosin (H&E) and Sirius Red. Samples are scored for NAS and fibrosis stage (outlined below) using of the clinical criteria outlined by Kleiner et al. 2005. Total NAS score represents the sum of scores for steatosis, inflammation, and ballooning, and ranges from 0-8.

TABLE 9
Total NAS scoring

| Feature | Degree | Score |
|---|---|---|
| Steatosis | <5% | 0 |
| | 5-33% | 1 |
| | >33-66% | 2 |
| | >66% | 3 |
| Lobular | No foci | 0 |
| inflammation | <2 foci/200x | 1 |
| | 2-4 foci/200x | 2 |
| | >4 foci/200x | 3 |
| Ballooning | None | 0 |
| degeneration | Few | 1 |
| | Many cells/prominent ballooning | 2 |
| Fibrosis | None | 0 |
| | Perisinusoidal or periportal | 1 |
| | Perisinusoidal & portal/periportal | 2 |
| | Bridging fibrosis | 3 |
| | Cirrhosis | 4 |

Adopted from: Design and validation of a histological scoring system or nonalcoholic fatty liver disease, Kleiner et al., Hepatology 41; 2005.

For lobular inflammation, inflammation is evaluated by counting the number of inflammatory foci per field using a 200× magnification (min. 5 fields per animal). A focus is defined as a cluster, not a row, of >3 inflammatory cells. Acidophil bodies are not included in this assessment, nor is portal inflammation. Fibrosis stage is evaluated separately from NAS.

### IHC and Steatosis Quantification

Quantitative assessment of immunoreactivity is evaluated as follows. IHC-positive staining is quantified by image analysis using the Visiomorph software (Visiopharm, Denmark). Visiomorph protocols are designed to analyze the virtual slides in two steps: 1. Crude detection of tissue at low magnification (1× objective). The liver capsule is excluded. 2. Detection of IHC-positive staining (e.g. green; collagen 1 IHC), tissue (e.g. red) and fat (e.g. pink) at high magnification (10× objective). The quantitative estimate of IHC-positive staining is calculated as an area fraction (AF) according to the following formula:

$${AF}_{IHC - pos} = \frac{{Area}_{IHC - pos}}{{Area}_{fat} + {Area}_{tissue} + {Area}_{IHC - pos}}$$

Quantitative assessment of steatosis is evaluated as follows. Steatosis is quantified on H&E stained slides by image analysis using the Visiomorph software (Visiopharm, Denmark). Visiomorph protocols are designed to analyze the virtual slides in two steps: 1. Crude detection of tissue at low magnification (1× objective). 2. Detection of steatosis (pink) and tissue (blue) at high magnification (20× objective). The quantitative estimate of steatosis is calculated as an area fraction (AF) according to the following formula:

$${AF}_{steatosis} = \frac{{Area}_{steatosis}}{{Area}_{tissue} + {Area}_{steatosis}}$$

### Example 2

This study assesses the effects of treatment with CHS-131 (Compound of Formula (I)), alone and in combination with other therapeutic agents, to treat NASH. Metabolic parameters, hepatic pathology, and NAFLD Activity Score including fibrosis stage are evaluated in male DIO-NASH mice.

Abbreviations used herein include: Alanine aminotransferase (ALT), Amylin liver NASH (AMLN), Aspartate aminotransferase (AST), Body weight (BW), Carboxy Methylcellulose CMC( ), Collagen 1A1 (Col1a1), Diet Induced obesity (DIO), Galectin-3 (Gal-3), Hematoxylin & Eosin (HE), Immunohistochemistry (IHC), Hydroxyproline (HP), Nonalcoholic fatty liver disease (NAFLD), NAFLD Activity Score (NAS), Nonalcoholic steatohepatitis (NASH), Per oral (PO), Total cholesterol (TC), Triglycerides (TG), Alpha-smooth muscle actin (α-SMA).

### Materials and Methods

### Mouse Model, NASH Induction and Randomization

### Mouse Strain

The animals used were male C57BL/6JRj mice supplied by JanVier (France) at 5 weeks of age.

### NASH Induction

The Diet-induced-obesity (DIO)-NASH mouse model was induced by feeding male C57BL/6JRj mice a high fat diet containing 40% fat with trans-fat, 20% fructose and 2% cholesterol (AMLN diet or D09100301, Research Diets Inc., USA). Induction of NASH was started at 5 weeks of age and mice were fed the AMLN diet for 36 weeks prior to study start resulting in NASH, which was confirmed by pre-biopsy prior to study start as described below.

### Pre-Biopsy Procedure and Randomization

Three weeks prior to study start, a pre-biopsy was performed to confirm NASH and for study inclusion of NASH-affected mice only. Briefly, mice were anesthetized with isoflurane (2-3%) in 100% oxygen. A small abdominal incision was made in the midline and the left lateral lobe of the liver exposed. A cone shaped wedge of liver tissue (approximately 50 mg) was excised from the distal portion of the lobe and fixated in 10% neutral buffered formalin (4% formaldehyde) for histopathological analyses. The cut surface of the liver was instantly electro-coagulated using bipolar coagulation (ERBE VIO 100 electrosurgical unit). The liver was returned to the abdominal cavity, the abdominal wall sutured, and the skin closed with staplers. For post-operative recovery, mice received carprofen (5 mg/ml-0.01 ml/10 g) administered subcutaneously on the day of operation and on post-operation day 1 and 2.

After surgery, the animals were evaluated daily on general health and body weight. In previous evaluations, an animal having sham surgery (just the abdominal incision) had the same body weight loss as an animal with a liver biopsy; around 10%. No evidence of greater pain (visceral pain) were observed in the animals where a biopsy was taken, compared to sham-operated animals. Signs of concerning pain or suffering has not been observed previously, and no animals had to be terminated (internal observations). The pre-biopsy was analyzed to evaluate liver steatosis score and fibrosis stage for study inclusion as outlined by Kleiner et al. (2005) (Table 1). In addition, liver Collagen 1a1 (Col1a1) quantified by morphometry was used to perform a stratified randomization of NASH-affected animals into study groups (see description of histopathological stains and analyses below).

### Formulation of Compounds

### Test Substances

CHS-131, Empagliflozin (Jardiance), and Elafibranor were suspended in 1% Methyl cellulose (MC) in deionized water. Test substance suspensions for dosing were prepared weekly and was protected from light. Liraglutide (Victoza) was suspended in PBS and prepared daily just before dosing.

### Route and Dose of Drug Administration

CHS-131 was administered at a dose of 10 mg/kg (low) or 30 mg/kg (high) once a day (AM).

Empagliflozin was administered at a dose of 10 mg/kg once a day (AM).

Elafibranor was administered at a dose of 30 mg/kg once a day (AM).

Liraglutide was administered at a dose of 0.4 mg/kg once a day (AM).

All compounds were administered at dose volume of 5 ml/kg. Vehicle, CHS-131, Empagliflozin and Elafibranor were administered per oral (PO) using oral gavage that was passed through the mouth into the stomach, where the drug suspension was deposited. The suspensions were stirred for 60 minutes before and during dosing. Liraglutide was administered subcutaneously (SC) once a day (AM).

### Tolerance Tests

### Intraperitoneal Insulin Tolerance Test

Mice were fasted 6 hours prior to intraperitoneal insulin administration (0.5 Unit/kg, rapid acting insulin NovoRapid). At the various time points after insulin administration, blood samples were collected into heparinized glass capillary tubes and immediately suspended in glucose/lactate system solution buffer (EKF-diagnostics, Germany). Blood glucose (BG) was measured using a BIOSEN c-Line glucose meter (EKF-diagnostics, Germany) according to the manufacturer's instructions. After the last blood sample, the animals were returned to the normal feeding schedule. The order of the animals was randomized before the procedure and mice were dosed with compounds just after the −60 minutes blood sample.

### Oral Glucose Tolerance Test

Animals were fasted 6 hours prior to oral glucose administration (2 g/kg). At the various time points after glucose administration, blood samples were collected into heparinized glass capillary tubes and immediately suspended in glucose/lactate system solution buffer (EKF-diagnostics, Germany). Blood glucose (BG) was measured using a BIOSEN c-Line glucose meter (EKF-diagnostics, Germany) according to the manufacturer's instructions. After the last blood sample, the animals were returned to the normal feeding schedule. The order of the animals were randomized before the procedure and mice were dosed with compounds just after the −60 minutes blood sample.

### EchoMRI Body Composition

The body composition of the mice was assessed by an EchoMRI 3-1 Body composition analyzer (EchoMRI, US). Non-anaesthetised mice was placed in a plastic tube inside the MRI scanner for approximately 80 seconds. The body composition is expressed as fat mass, fat free mass (lean mass) and water.

### Termination and Sample Collection

### Blood Sampling and Plasma Preparation

For plasma biochemistry, tail blood was drawn directly through the capillary of a Microvette/Vacuette of the right dimension and anticoagulant and mixed by inversion 5 times. Blood was placed at 4° C. until centrifugation at 3000×g for 10 minutes at 4° C. The plasma supernatants were transferred to new tubes and immediately frozen on dry ice and stored at −80° C. until analysis.

### Termination

Animals were terminated after 12 weeks of treatment in a non-fasting state. Animals were put under isoflurane anesthesia, the abdominal cavity was opened, and cardiac blood was drawn directly into a Vacuette of the right dimension and anticoagulant and mixed by inversion 5 times. Blood was placed at 4° C. until centrifugation at 3000×g for 10 minutes at 4° C. The plasma supernatants were transferred to new tubes and immediately frozen on dry ice and stored at −80° C. Upon necropsy, the whole liver was collected and weighed. The liver was sampled for histological and biochemical analyses as described below.

### Liver Sampling and Sample Preparation

The liver post-biopsy for histological analyses was removed by dissection from the left lateral lobe, fixated in 4% formalin for 20-24 h, and subsequently embedded in paraffin. Liver biopsies for liver triglycerides and total cholesterol were dissected from the medial lobe, snap frozen in liquid nitrogen, and stored at −80° C., while liver biopsies for hydroxyproline were dissected from the caudal lobe (the entire lobe), snap frozen in liquid nitrogen and stored at −80° C. Finally, a liver sample for RNA isolation and gene expression analysis was dissected from the left lateral lobe, snap frozen in liquid nitrogen, and stored at −80° C. until processing.

### Measurement of Plasma and Liver Biochemistry

### Measurement of Plasma Biochemistry

Plasma alanine transaminase (ALT) (Roche Diagnostics), aspartate transaminase (AST) (Roche Diagnostics), triglycerides (TG) (Roche Diagnostics), total cholesterol (TC) (Roche Diagnostics), creatinine (Roche Diagnostics), and urea (Roche Diagnostics) were measured using commercial kits on the Cobas c 501 autoanalyzer according to the manufacturer's instructions. Mouse insulin was measured in single determinations using the MSD platform (Meso Scale Diagnostics).

### Measurement of Liver Biochemistry

For liver hydroxyproline (HP; a protein marker of fibrosis) quantification, liver samples were homogenized in 6 M HCl and hydrolyzed to degrade collagen. The samples were centrifuged, and the hydroxyproline content measured in duplicates in the supernatant, using a colorimetric assay (Quickzyme Biosciences) according to the manufacturer's instructions.

For liver TG and TC quantification, samples were homogenized, and TG and TC extracted in 5% NP-40 by heating twice to 90° C. The samples were centrifuged, and the TG and TC content measured in the supernatant, using commercial kits (Roche Diagnostics) on the Cobas c501 autoanalyzer according to the manufacturer's instructions.

### Histological Tissue Preparation and Staining Procedures

### Histological Tissue Preparation

Liver biopsies fixated in formalin were infiltrated over-night in paraffin in an automated Miles Scientific Tissue-TEK VIP Tissue Processor and subsequently embedded in paraffin blocks, which were trimmed and from which 3 μm thick sections were cut on a Microm HM340E Microtome. Slides with paraffin-embedded sections were de-paraffinated in xylene and rehydrated in a series of graded ethanol prior to histochemical or immunohistochemical (IHC) staining.

### Histochemical Stains

For Hematoxylin & Eosin (HE) staining, slides were incubated in Mayer's Hematoxylin, washed in tap water, stained in Eosin Y solution, hydrated, mounted with Pertex and allowed to dry before scanning.

For Sirius red staining, slides were incubated in Weigert's iron hematoxylin, washed in tap water, stained in Picro-Sirius red and washed twice in acidified water. Excess water was removed by shaking the slides after which the slides were dehydrated in three changes of 100% ethanol, cleared in xylene, mounted with Pertex and allowed to dry before scanning.

### Immunohistochemical Stains

Protein markers of fibrosis (Col1a1), fibrogenesis (α-SMA) and inflammation (Gal-3) were assessed by immunohistochemistry. α-SMA and collagen type I increase in regulation of quiescent hepatic stellate cell activation into myofibroblast-like cells where activated hepatic stellate cells are the main collagen producing cells in the liver (Carpino et al 2005, Hou and Syn 2018) whereas Gal-3 is involved in mediating inflammatory response and considered as a macrophage activation marker (Sciacchitano et al, 2018). For morphometric quantification of liver Col1a1 (using antibody from Southern Biotech, Cat. #1310-01), alpha-smooth muscle actin (α-SMA; using antibody from Abcam, Cat. #Ab124964) and Galectin-3 (using antibody from Biolegend, Cat. #125402), IHC staining was performed using standard procedures. Briefly, after antigen retrieval and blocking of endogenous peroxidase activity, slides were incubated with primary antibody. For all IHC stains, the primary antibody was detected using a polymeric HRP-linker antibody conjugate and visualized using DAB as chromogen. Finally, sections were counterstained in hematoxylin and cover-slipped before scanning.

### NAFLD Activity Score and Fibrosis Stage

For scoring of NAFLD Activity Score (NAS) and fibrosis stage, HE and Sirius red stained liver sections, respectively, were scored by a histopathology specialist as outlined in Table 10 using the clinical criteria outlined by Kleiner et al. (2005). Total NAS score represents the sum of scores for steatosis, lobular inflammation, and ballooning degeneration scores, and ranges from 0-8.

TABLE 10

| Feature | Degree | Score |
|---|---|---|
| Steatosis | <5% | 0 |
| | 5-33% | 1 |
| | >33-66% | 2 |
| | >66% | 3 |
| Lobular | No foci | 0 |
| inflammation | <2 foci/200x | 1 |
| | 2-4 foci/200x | 2 |
| | >4 foci/200x | 3 |
| Ballooning | None | 0 |
| degeneration | Few | 1 |
| | Many cells/prominent ballooning | 2 |
| Fibrosis | None | 0 |
| | Perisinusoidal or periportal | 1 |
| | Perisinusoidal & portal/periportal | 2 |
| | Bridging fibrosis | 3 |
| | Cirrhosis | 4 |

For steatosis score, percentage refers to percentage of hepatocytes affected by steatosis as evaluated on low to medium power examination.

For lobular inflammation, inflammation is evaluated by counting the number of inflammatory foci per field using a 200× magnification (min. 5 fields per animal). A focus is defined as a cluster, not a row, of >3 inflammatory cells. Acidophil bodies are not included in this assessment, nor is portal inflammation.

For hepatocellular ballooning degeneration, degenerated hepatocytes with a cleared cytoplasm, enlargement, swelling, rounding and reticulated cytoplasm were identified.

Fibrosis stage is evaluated separately from NAS.

IHC and Steatosis Quantification

Quantitative assessment of immunoreactivity is evaluated as follows. IHC-positive staining is quantified by image analysis using the Visiomorph software (Visiopharm, Denmark). Visiomorph protocols are designed to analyze the virtual slides in two steps: 1. Crude detection of tissue at low magnification (1× objective). The liver capsule is excluded. 2. Detection of IHC-positive staining (e.g. green; collagen 1 IHC), tissue (e.g. red) and fat (e.g. pink) at high magnification (10×objective). The quantitative estimate of IHC-positive staining is calculated as an area fraction (AF) according to the following formula:

$${AF}_{IHC - pos} = \frac{{Area}_{IHC - pos}}{{Area}_{fat} + {Area}_{tissue} + {Area}_{IHC - pos}}$$

Quantitative assessment of steatosis is evaluated as follows. Steatosis is quantified on H&E stained slides by image analysis using the Visiomorph software (Visiopharm, Denmark). Visiomorph protocols are designed to analyze the virtual slides in two steps: 1. Crude detection of tissue at low magnification (1× objective). 2. Detection of steatosis (pink) and tissue (blue) at high magnification (20× objective). The quantitative estimate of steatosis is calculated as an area fraction (AF) according to the following formula:

$${AF}_{steatosis} = \frac{{Area}_{steatosis}}{{Area}_{tissue} + {Area}_{steatosis}}$$

### Statistical Tests

For single-timepoint continuous data, the data were fitted to a one-factor linear regression model with the treatment groups as categorical, independent (predictor) variables and Dunnett's test was used to compare treatments to the Vehicle control. The reported p-values are one-tailed unless otherwise stated in the figure legend. Data from categorical endpoints, such as histopathological scoring values, were partitioned into a 2×2 contingency table containing responders and non-responders in control and treatment groups. Fisher's exact test assumes that contingency table row and column totals are fixed, sampling is random, and observations can be classified only into one cell. Comparison of data and significance different for multiple testing is visualized on the graph when p<0.05.

The data is presented in three different ways.
a) All groups (group 1-9)
b) Orally treated groups only (group 1-6+9)
c) Focused analysis (group 1-6)

### Randomization—Col1a1(%)

Liver fibrosis (Col1a1%) at baseline. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 5.04 | 1.12 | 0.323 | 0.71 |
| NASH vehicle + vehicle | 14 | 19.9 | 4.79 | 1.28 | 2.77 |
| CHS-131 Low + Vehicle | 14 | 19.7 | 4.26 | 1.14 | 2.46 |
| CHS-131 High + Vehicle | 14 | 19.8 | 3.81 | 1.02 | 2.2 |
| Vehicle + Empagliflozin | 14 | 19.8 | 3.61 | 0.965 | 2.09 |
| CHS-131 High + | 14 | 19.8 | 3.35 | 0.896 | 1.94 |
| Empagliflozin | | | | | |
| Vehicle + Liraglutide | 14 | 19.7 | 3.36 | 0.899 | 1.94 |
| CHS-131 High + | 14 | 19.8 | 3.29 | 0.878 | 1.9 |
| Liraglutide | | | | | |
| Elafibranor + Vehicle | 14 | 19.8 | 3.28 | 0.877 | 1.9 |

### Effects of Treatments on Absolute Body Weight

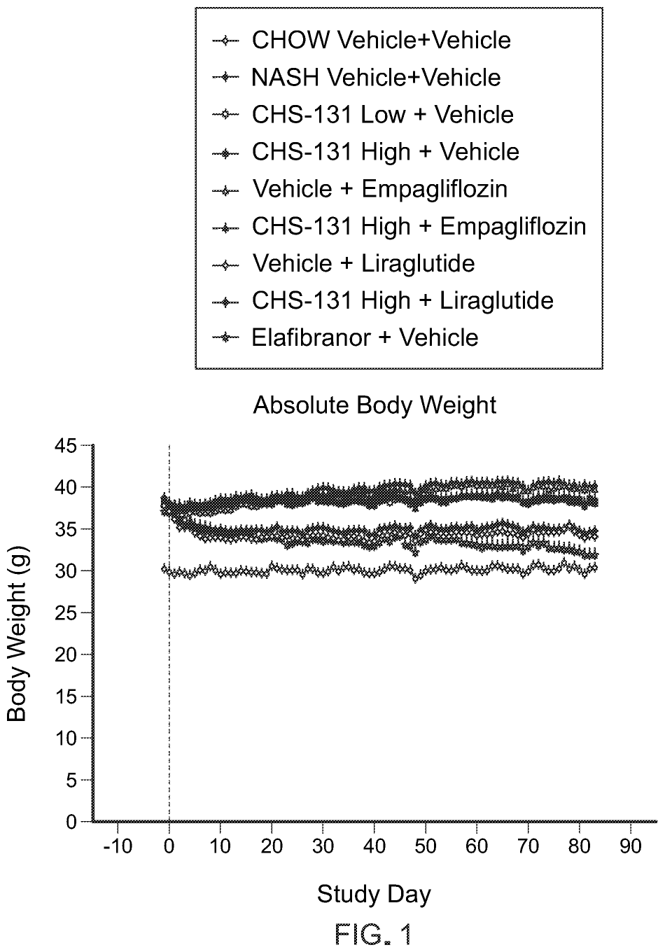

Absolute body weight, BW, (g) was measured daily throughout the study period. Values expressed as mean of n=11-14+SEM (see FIG. 1).

Absolute body weight at termination (measured day 83/84/85). Values expressed as mean of n=12-14+SEM.

| | | Average | | | 95% Conf. |
|---|---|---|---|---|---|
| Treatment | N | (grams) | Std. dev. | SEM | int. |
| CHOW vehicle + vehicle | 12 | 30.4 | 2.13 | 0.614 | 1.35 |
| NASH vehicle + vehicle | 13 | 39.9 | 2.12 | 0.587 | 1.28 |
| CHS-131 Low + Vehicle | 13 | 38.2 | 1.94 | 0.538 | 1.17 |
| CHS-131 High + Vehicle | 13 | 38.2 | 3.37 | 0.935 | 2.04 |
| Vehicle + Empagliflozin | 13 | 39.6 | 1.99 | 0.553 | 1.2 |
| CHS-131 High + | 14 | 38.1 | 3.44 | 0.92 | 1.99 |
| Empagliflozin | | | | | |
| Vehicle + Liraglutide | 14 | 34.1 | 2.28 | 0.609 | 1.32 |
| CHS-131 High + | 13 | 34.8 | 1.94 | 0.538 | 1.17 |
| Liraglutide | | | | | |
| Elafibranor + Vehicle | 13 | 31.8 | 3.45 | 0.957 | 2.08 |

### Effects of Treatments on Relative Body Weight

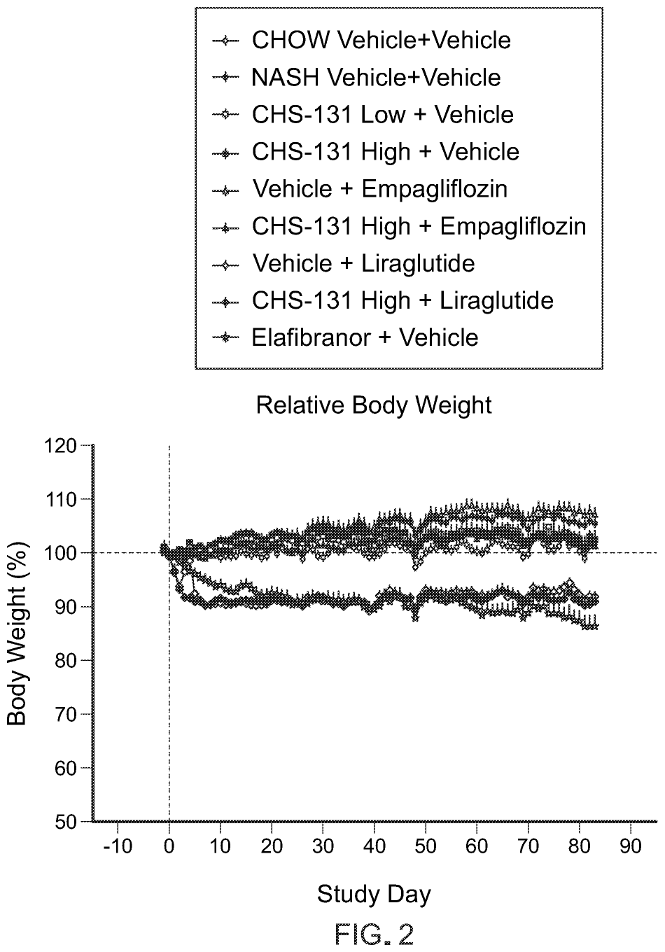

Relative BW, recorded as percentage of starting body weight (baseline day 0=100%), was measured daily throughout the study period. Values expressed as mean of n=11-14+SEM (see FIG. 2).

Terminal relative body weight (measured day 83/84/85), recorded as percentage of starting body weight (baseline day 0=100%). Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 102 | 2.08 | 0.601 | 1.32 |
| NASH vehicle + vehicle | 13 | 105 | 3.81 | 1.06 | 2.3 |
| CHS-131 Low + Vehicle | 13 | 103 | 3.55 | 0.985 | 2.15 |
| CHS-131 High + Vehicle | 13 | 102 | 7.21 | 2 | 4.36 |
| Vehicle + Empagliflozin | 13 | 107 | 5.7 | 1.58 | 3.45 |
| CHS-131 High + | 14 | 101 | 4.62 | 1.24 | 2.67 |
| Empagliflozin | | | | | |
| Vehicle + Liraglutide | 14 | 91.3 | 5.14 | 1.37 | 2.97 |
| CHS-131 High + | 13 | 91.1 | 3.39 | 0.939 | 2.05 |
| Liraglutide | | | | | |
| Elafibranor + Vehicle | 13 | 86.5 | 7.47 | 2.07 | 4.51 |

### Daily and Cumulative Food Intake

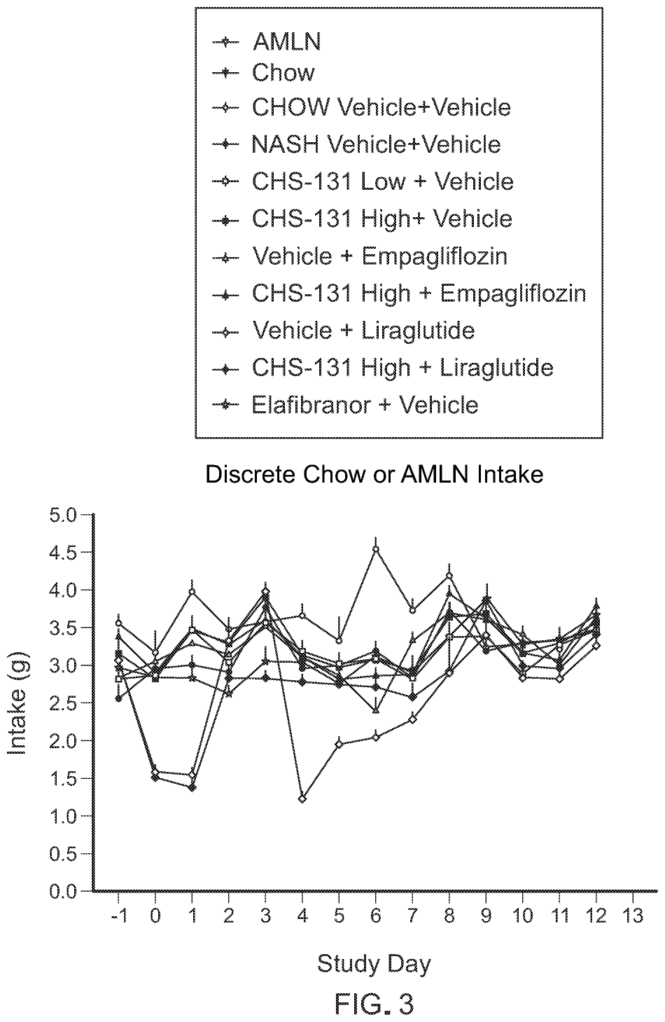

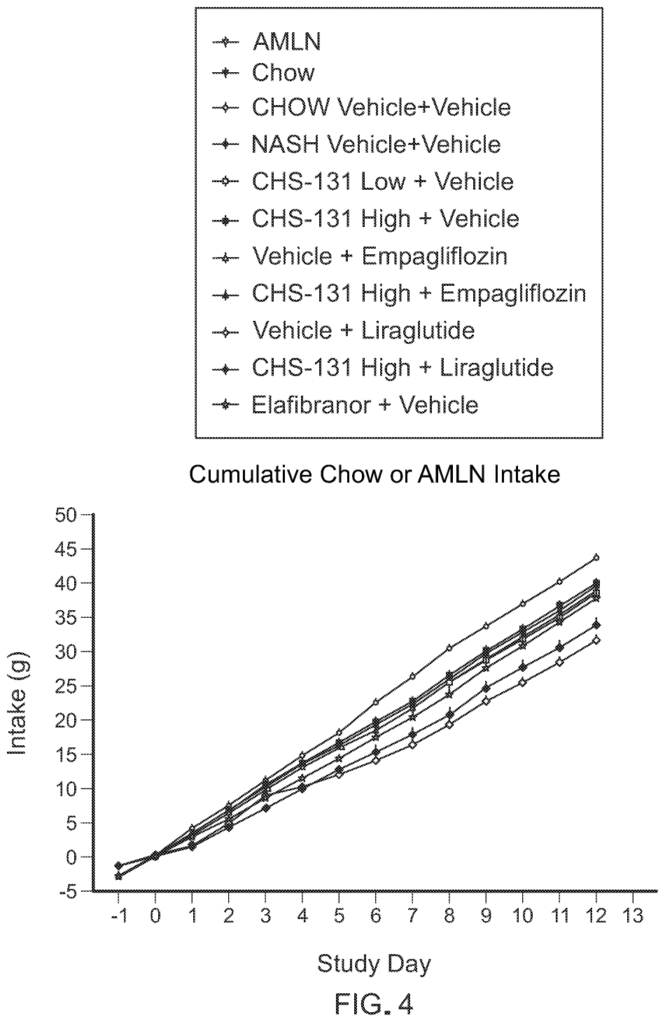

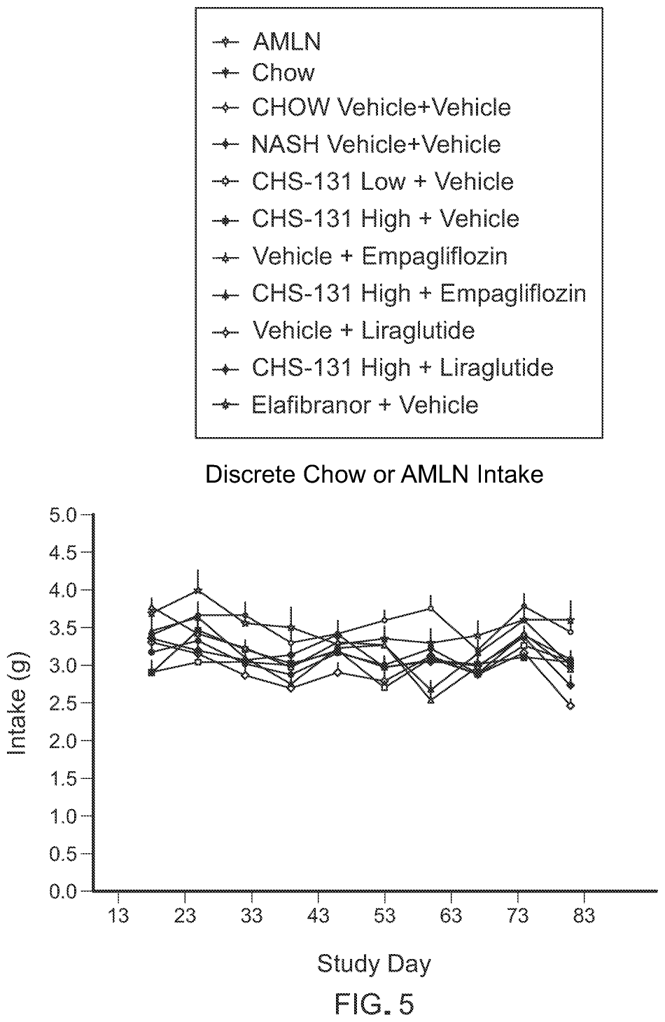

Discrete daily food intake (g) during week 1-2 of treatment. Values expressed as mean of n=12-14+SEM (FIG. 3). Cumulative daily food intake (g) during week 1-2 of treatment. Values expressed as mean of n=12-14+SEM (FIG. 4). Weekly food intake (g) during week 3-12 of treatment. Values expressed as mean of n=12-14+SEM (FIG. 5).

### Effects of Treatments on MRI Body Weight at Baseline and Week 11

MRI body weight (g) at baseline. Values are expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 30.2 | 2.27 | 0.656 | 1.44 |
| NASH vehicle + vehicle | 13 | 38.5 | 2.24 | 0.622 | 1.36 |
| CHS-131 Low + Vehicle | 13 | 37.6 | 2.58 | 0.717 | 1.56 |
| CHS-131 High + Vehicle | 13 | 37.7 | 1.94 | 0.537 | 1.17 |
| Vehicle + Empagliflozin | 13 | 37.4 | 2.36 | 0.653 | 1.42 |
| CHS-131 High + | 14 | 37.8 | 3.16 | 0.845 | 1.83 |
| Empagliflozin | | | | | |
| Vehicle + Liraglutide | 14 | 37.6 | 2.49 | 0.665 | 1.44 |
| CHS-131 High + | 13 | 38.7 | 1.74 | 0.482 | 1.05 |
| Liraglutide | | | | | |
| Elafibranor + Vehicle | 13 | 36.9 | 3.5 | 0.97 | 2.11 |

MRI body weight (g) at week 11. Values are expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 30.2 | 1.99 | 0.573 | 1.26 |
| NASH vehicle + vehicle | 13 | 40.1 | 2.21 | 0.614 | 1.34 |
| CHS-131 Low + Vehicle | 13 | 38.3 | 2.15 | 0.596 | 1.3 |
| CHS-131 High + Vehicle | 13 | 38.2 | 3.53 | 0.979 | 2.13 |
| Vehicle + Empagliflozin | 13 | 40.1 | 2.18 | 0.603 | 1.31 |
| CHS-131 High + | 14 | 38.8 | 3.42 | 0.914 | 1.97 |
| Empagliflozin | | | | | |
| Vehicle + Liraglutide | 14 | 35.3 | 1.83 | 0.49 | 1.06 |
| CHS-131 High + | 13 | 35.4 | 2.12 | 0.589 | 1.28 |
| Liraglutide | | | | | |
| Elafibranor + Vehicle | 13 | 32.5 | 3.62 | 1 | 2.19 |

Effects of Treatments on Absolute Fat Tissue Mass

Absolute fat tissue mass (g) at baseline. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 2.36 | 0.752 | 0.217 | 0.478 |
| NASH vehicle + vehicle | 13 | 7.92 | 1.85 | 0.513 | 1.12 |
| CHS-131 Low + Vehicle | 13 | 6.89 | 1.2 | 0.332 | 0.723 |
| CHS-131 High + Vehicle | 13 | 6.83 | 1.73 | 0.48 | 1.05 |
| Vehicle + Empagliflozin | 13 | 6.2 | 1.42 | 0.395 | 0.86 |
| CHS-131 High + | 14 | 6.52 | 1.56 | 0.418 | 0.903 |
| Empagliflozin | | | | | |
| Vehicle + Liraglutide | 14 | 6.82 | 1.57 | 0.419 | 0.906 |
| CHS-131 High + | 13 | 7.11 | 1.08 | 0.301 | 0.656 |
| Liraglutide | | | | | |
| Elafibranor + Vehicle | 13 | 6.41 | 1.89 | 0.525 | 1.14 |

Absolute fat tissue mass (g) at week 11. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 2.67 | 0.848 | 0.245 | 0.539 |
| NASH vehicle + vehicle | 13 | 8.95 | 1.82 | 0.504 | 1.1 |
| CHS-131 Low + Vehicle | 13 | 7.07 | 1.42 | 0.393 | 0.857 |
| CHS-131 High + Vehicle | 13 | 6.9 | 2.26 | 0.628 | 1.37 |
| Vehicle + Empagliflozin | 13 | 7.81 | 1.78 | 0.495 | 1.08 |
| CHS-131 High + | 14 | 7.02 | 1.89 | 0.506 | 1.09 |
| Empagliflozin | | | | | |
| Vehicle + Liraglutide | 14 | 4.92 | 0.833 | 0.223 | 0.481 |
| CHS-131 High + | 13 | 4.73 | 1.25 | 0.347 | 0.755 |
| Liraglutide | | | | | |
| Elafibranor + Vehicle | 13 | 3.73 | 1.11 | 0.308 | 0.671 |

Delta fat tissue mass (g) week 11 vs baseline. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 0.312 | 0.601 | 0.174 | 0.382 |
| NASH vehicle + vehicle | 13 | 1.03 | 1.49 | 0.413 | 0.899 |
| CHS-131 Low + Vehicle | 13 | 0.182 | 0.91 | 0.252 | 0.55 |
| CHS-131 High + Vehicle | 13 | 0.0685 | 2.01 | 0.557 | 1.21 |
| Vehicle + Empagliflozin | 13 | 1.6 | 1.86 | 0.515 | 1.12 |
| CHS-131 High + | 14 | 0.504 | 1.18 | 0.317 | 0.684 |
| Empagliflozin | | | | | |
| Vehicle + Liraglutide | 14 | −1.9 | 1.51 | 0.403 | 0.871 |
| CHS-131 High + | 13 | −2.39 | 1.23 | 0.34 | 0.741 |
| Liraglutide | | | | | |
| Elafibranor + Vehicle | 13 | −2.67 | 1.66 | 0.461 | 1 |

### Effects of Treatments on Relative Fat Tissue Mass

Relative fat tissue mass (% of body weight) at baseline. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 7.78 | 2.05 | 0.592 | 1.3 |
| NASH vehicle + vehicle | 13 | 20.5 | 4.01 | 1.11 | 2.42 |
| CHS-131 Low + Vehicle | 13 | 18.3 | 2.28 | 0.631 | 1.38 |
| CHS-131 High + Vehicle | 13 | 18.1 | 4.33 | 1.2 | 2.62 |
| Vehicle + Empagliflozin | 13 | 16.5 | 3.16 | 0.876 | 1.91 |
| CHS-131 High + | 14 | 17.2 | 3.48 | 0.929 | 2.01 |
| Empagliflozin | | | | | |
| Vehicle + Liraglutide | 14 | 18 | 2.91 | 0.778 | 1.68 |
| CHS-131 High + | 13 | 18.3 | 2.26 | 0.626 | 1.36 |
| Liraglutide | | | | | |
| Elafibranor + Vehicle | 13 | 17.1 | 3.64 | 1.01 | 2.2 |

Relative fat tissue mass (% of body weight) at week 11 (top-right). Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 8.74 | 2.24 | 0.645 | 1.42 |
| NASH vehicle + vehicle | 13 | 22.2 | 3.47 | 0.962 | 2.1 |
| CHS-131 Low + Vehicle | 13 | 18.4 | 2.69 | 0.747 | 1.63 |
| CHS-131 High + Vehicle | 13 | 17.8 | 5.13 | 1.42 | 3.1 |
| Vehicle + Empagliflozin | 13 | 19.4 | 3.89 | 1.08 | 2.35 |
| CHS-131 High + | 14 | 17.9 | 3.55 | 0.949 | 2.05 |
| Empagliflozin | | | | | |
| Vehicle + Liraglutide | 14 | 13.9 | 2.2 | 0.588 | 1.27 |
| CHS-131 High + | 13 | 13.2 | 2.96 | 0.82 | 1.79 |
| Liraglutide | | | | | |
| Elafibranor + Vehicle | 13 | 11.3 | 2.5 | 0.692 | 1.51 |

Delta fat tissue mass (0% of body weight) week 11 vs baseline. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 0.958 | 1.99 | 0.574 | 1.26 |
| NASH vehicle + vehicle | 13 | 1.74 | 3.09 | 0.856 | 1.86 |
| CHS-131 Low + Vehicle | 13 | 0.0932 | 2.18 | 0.605 | 1.32 |
| CHS-131 High + Vehicle | 13 | −0.322 | 4.38 | 1.21 | 2.64 |
| Vehicle + Empagliflozin | 13 | 2.87 | 4.17 | 1.16 | 2.52 |
| CHS-131 High + | 14 | 0.724 | 2.75 | 0.736 | 1.59 |
| Empagliflozin | | | | | |
| Vehicle + Liraglutide | 14 | −4.05 | 3.21 | 0.857 | 1.85 |
| CHS-131 High + | 13 | −5.1 | 2.98 | 0.825 | 1.8 |
| Liraglutide | | | | | |
| Elafibranor + Vehicle | 13 | −5.84 | 3.86 | 1.07 | 2.33 |

### Effects of Treatments on Absolute Lean Tissue Mass.

Absolute lean tissue mass (g) at baseline. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 16.4 | 1.89 | 0.547 | 1.2 |
| NASH vehicle + vehicle | 13 | 19.1 | 0.885 | 0.245 | 0.535 |
| CHS-131 Low + Vehicle | 13 | 18.9 | 1.83 | 0.508 | 1.11 |
| CHS-131 High + Vehicle | 13 | 19.8 | 1.59 | 0.442 | 0.962 |
| Vehicle + Empagliflozin | 13 | 19 | 1.45 | 0.403 | 0.877 |
| CHS-131 High + | 14 | 18.5 | 2.38 | 0.636 | 1.37 |
| Empagliflozin | | | | | |
| Vehicle + Liraglutide | 14 | 18.6 | 1.73 | 0.463 | 1 |
| CHS-131 High + | 13 | 19.6 | 1.67 | 0.462 | 1.01 |
| Liraglutide | | | | | |
| Elafibranor + Vehicle | 13 | 19 | 2.31 | 0.64 | 1.4 |

Absolute lean tissue mass (g) at week 11. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 55.6 | 3.39 | 0.98 | 2.16 |
| NASH vehicle + vehicle | 13 | 47.8 | 4.18 | 1.16 | 2.53 |
| CHS-131 Low + Vehicle | 13 | 48.5 | 3.63 | 1.01 | 2.2 |
| CHS-131 High + Vehicle | 13 | 48.1 | 3.62 | 1.01 | 2.19 |
| Vehicle + Empagliflozin | 13 | 47.1 | 4.6 | 1.28 | 2.78 |
| CHS-131 High + | 14 | 48.1 | 3.67 | 0.981 | 2.12 |
| Empagliflozin | | | | | |
| Vehicle + Liraglutide | 14 | 50.1 | 4.92 | 1.31 | 2.84 |
| CHS-131 High + | 13 | 50.5 | 3.64 | 1.01 | 2.2 |
| Liraglutide | | | | | |
| Elafibranor + Vehicle | 13 | 52.6 | 3.98 | 1.1 | 2.41 |

Delta lean tissue mass (g) week 11 vs baseline. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 0.395 | 1.64 | 0.472 | 1.04 |
| NASH vehicle + vehicle | 13 | 0.0762 | 2.13 | 0.592 | 1.29 |
| CHS-131 Low + Vehicle | 13 | −0.396 | 1.71 | 0.474 | 1.03 |
| CHS-131 High + Vehicle | 13 | −1.43 | 2.31 | 0.642 | 1.4 |
| Vehicle + Empagliflozin | 13 | −0.12 | 2.34 | 0.648 | 1.41 |
| CHS-131 High + | 14 | 0.2 | 1.5 | 0.4 | 0.865 |
| Empagliflozin | | | | | |
| Vehicle + Liraglutide | 14 | −1.03 | 2.02 | 0.539 | 1.16 |
| CHS-131 High + | 13 | −1.72 | 1.79 | 0.496 | 1.08 |
| Liraglutide | | | | | |
| Elafibranor + Vehicle | 13 | −1.9 | 1.98 | 0.549 | 1.2 |

### Effects of Treatments on Relative Lean Tissue Mass

Relative fat tissue mass (% of body weight) at baseline. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 54.4 | 3.86 | 1.11 | 2.45 |
| NASH vehicle + vehicle | 13 | 49.8 | 3.27 | 0.906 | 1.97 |
| CHS-131 Low + Vehicle | 13 | 50.5 | 4.48 | 1.24 | 2.71 |
| CHS-131 High + Vehicle | 13 | 52.5 | 3.91 | 1.09 | 2.36 |
| Vehicle + Empagliflozin | 13 | 51 | 4.48 | 1.24 | 2.71 |
| CHS-131 High + | 14 | 48.8 | 4.85 | 1.3 | 2.8 |
| Empagliflozin | | | | | |
| Vehicle + Liraglutide | 14 | 49.7 | 4.36 | 1.16 | 2.51 |
| CHS-131 High + | 13 | 50.6 | 3.29 | 0.914 | 1.99 |
| Liraglutide | | | | | |
| Elafibranor + Vehicle | 13 | 51.4 | 4.5 | 1.25 | 2.72 |

Relative fat tissue mass (% of body weight) at week 11. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 55.6 | 3.39 | 0.98 | 2.16 |
| NASH vehicle + vehicle | 13 | 47.8 | 4.18 | 1.16 | 2.53 |
| CHS-131 Low + Vehicle | 13 | 48.5 | 3.63 | 1.01 | 2.2 |
| CHS-131 High + Vehicle | 13 | 48.1 | 3.62 | 1.01 | 2.19 |
| Vehicle + Empagliflozin | 13 | 47.1 | 4.6 | 1.28 | 2.78 |
| CHS-131 High + | 14 | 48.1 | 3.67 | 0.981 | 2.12 |
| Empagliflozin | | | | | |
| Vehicle + Liraglutide | 14 | 50.1 | 4.92 | 1.31 | 2.84 |
| CHS-131 High + | 13 | 50.5 | 3.64 | 1.01 | 2.2 |
| Liraglutide | | | | | |
| Elafibranor + Vehicle | 13 | 52.6 | 3.98 | 1.1 | 2.41 |

Delta lean tissue mass (% of body weight) week 11 vs baseline. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 1.14 | 4.8 | 1.38 | 3.05 |
| NASH vehicle + vehicle | 13 | −1.95 | 5.44 | 1.51 | 3.28 |
| CHS-131 Low + Vehicle | 13 | −2 | 4.19 | 1.16 | 2.53 |
| CHS-131 High + Vehicle | 13 | −4.39 | 6.01 | 1.67 | 3.63 |
| Vehicle + Empagliflozin | 13 | −3.91 | 6.3 | 1.75 | 3.81 |
| CHS-131 High + | 14 | −0.682 | 4.59 | 1.23 | 2.65 |
| Empagliflozin | | | | | |
| Vehicle + Liraglutide | 14 | 0.404 | 4.71 | 1.26 | 2.72 |
| CHS-131 High + | 13 | −0.0905 | 4.29 | 1.19 | 2.6 |
| Liraglutide | | | | | |
| Elafibranor + Vehicle | 13 | 1.22 | 6.92 | 1.92 | 4.18 |

### Effects of Treatments on Absolute Free Water Mass

Absolute free water mass (g) at baseline. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 0.0917 | 0.0909 | 0.0263 | 0.0578 |
| NASH vehicle + vehicle | 13 | 0.0923 | 0.0924 | 0.0256 | 0.0558 |
| CHS-131 Low + Vehicle | 13 | 0.114 | 0.0961 | 0.0267 | 0.0581 |
| CHS-131 High + Vehicle | 13 | 0.103 | 0.117 | 0.0323 | 0.0704 |
| Vehicle + Empagliflozin | 13 | 0.128 | 0.141 | 0.039 | 0.085 |
| CHS-131 High + | 14 | 0.146 | 0.0907 | 0.0242 | 0.0524 |
| Empagliflozin | | | | | |
| Vehicle + Liraglutide | 14 | 0.167 | 0.141 | 0.0376 | 0.0812 |
| CHS-131 High + | 13 | 0.152 | 0.169 | 0.0468 | 0.102 |
| Liraglutide | | | | | |
| Elafibranor + Vehicle | 13 | 0.172 | 0.137 | 0.0379 | 0.0826 |

Absolute free water mass (g) at week 11. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 0.188 | 0.0729 | 0.021 | 0.0463 |
| NASH vehicle + vehicle | 13 | 0.132 | 0.163 | 0.0451 | 0.0983 |
| CHS-131 Low + Vehicle | 13 | 0.165 | 0.136 | 0.0378 | 0.0823 |
| CHS-131 High + Vehicle | 13 | 0.0946 | 0.106 | 0.0293 | 0.0638 |
| Vehicle + Empagliflozin | 13 | 0.182 | 0.147 | 0.0407 | 0.0886 |
| CHS-131 High + | 14 | 0.131 | 0.133 | 0.0355 | 0.0766 |
| Empagliflozin | | | | | |
| Vehicle + Liraglutide | 14 | 0.178 | 0.148 | 0.0394 | 0.0852 |
| CHS-131 High + | 13 | 0.138 | 0.117 | 0.0325 | 0.0707 |
| Liraglutide | | | | | |
| Elafibranor + Vehicle | 13 | 0.0923 | 0.116 | 0.0323 | 0.0703 |

### Effects of Treatments on Relative Free Water Mass

Relative free water mass (% of body weight) at baseline. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 0.305 | 0.305 | 0.088 | 0.194 |
| NASH vehicle + vehicle | 13 | 0.234 | 0.235 | 0.0652 | 0.142 |
| CHS-131 Low + Vehicle | 13 | 0.303 | 0.255 | 0.0708 | 0.154 |
| CHS-131 High + Vehicle | 13 | 0.267 | 0.292 | 0.0811 | 0.177 |
| Vehicle + Empagliflozin | 13 | 0.354 | 0.39 | 0.108 | 0.236 |
| CHS-131 High + | 14 | 0.388 | 0.234 | 0.0626 | 0.135 |
| Empagliflozin | | | | | |
| Vehicle + Liraglutide | 14 | 0.441 | 0.363 | 0.0969 | 0.209 |
| CHS-131 High + | 13 | 0.395 | 0.44 | 0.122 | 0.266 |
| Liraglutide | | | | | |
| Elafibranor + Vehicle | 13 | 0.464 | 0.37 | 0.103 | 0.224 |

Relative free water mass (% of body weight) at week 11. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 0.629 | 0.259 | 0.0748 | 0.165 |
| NASH vehicle + vehicle | 13 | 0.326 | 0.4 | 0.111 | 0.242 |
| CHS-131 Low + Vehicle | 13 | 0.429 | 0.364 | 0.101 | 0.22 |
| CHS-131 High + Vehicle | 13 | 0.249 | 0.271 | 0.0753 | 0.164 |
| Vehicle + Empagliflozin | 13 | 0.458 | 0.381 | 0.106 | 0.23 |
| CHS-131 High + | 14 | 0.336 | 0.342 | 0.0913 | 0.197 |
| Empagliflozin | | | | | |
| Vehicle + Liraglutide | 14 | 0.506 | 0.418 | 0.112 | 0.241 |
| CHS-131 High + | 13 | 0.388 | 0.332 | 0.092 | 0.2 |
| Liraglutide | | | | | |
| Elafibranor + Vehicle | 13 | 0.292 | 0.364 | 0.101 | 0.22 |

### Effects of Treatments on Fasted Blood Glucose

4 h fasted blood glucose (mmol/L) at baseline. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 7.46 | 0.633 | 0.183 | 0.402 |
| NASH vehicle + vehicle | 13 | 8.17 | 0.46 | 0.128 | 0.278 |
| CHS-131 Low + Vehicle | 13 | 8.09 | 0.489 | 0.136 | 0.296 |
| CHS-131 High + Vehicle | 13 | 7.68 | 0.62 | 0.172 | 0.375 |
| Vehicle + Empagliflozin | 13 | 7.71 | 0.954 | 0.265 | 0.576 |
| CHS-131 High + | 14 | 8.1 | 0.886 | 0.237 | 0.512 |
| Empagliflozin | | | | | |
| Vehicle + Liraglutide | 14 | 8.01 | 0.662 | 0.177 | 0.382 |
| CHS-131 High + | 13 | 7.99 | 0.442 | 0.123 | 0.267 |
| Liraglutide | | | | | |
| Elafibranor + Vehicle | 13 | 7.68 | 0.681 | 0.189 | 0.411 |

4 h fasted blood glucose (mmol/L) at week 12. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 7.77 | 0.467 | 0.135 | 0.297 |
| NASH vehicle + vehicle | 13 | 7.53 | 0.62 | 0.172 | 0.375 |
| CHS-131 Low + Vehicle | 13 | 7.31 | 0.749 | 0.208 | 0.452 |
| CHS-131 High + Vehicle | 13 | 7.01 | 0.728 | 0.202 | 0.44 |
| Vehicle + Empagliflozin | 13 | 6.69 | 0.471 | 0.131 | 0.285 |
| CHS-131 High + | 14 | 7.08 | 0.483 | 0.129 | 0.279 |
| Empagliflozin | | | | | |
| Vehicle + Liraglutide | 14 | 6.72 | 0.493 | 0.132 | 0.284 |
| CHS-131 High + | 13 | 7.42 | 0.504 | 0.14 | 0.305 |
| Liraglutide | | | | | |
| Elafibranor + Vehicle | 13 | 6.41 | 0.707 | 0.196 | 0.427 |

Delta 4 h fasted blood glucose (mmol/L) week 12 vs baseline (Bottom). Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 0.303 | 0.767 | 0.221 | 0.487 |
| NASH vehicle + vehicle | 13 | −0.638 | 0.832 | 0.231 | 0.503 |
| CHS-131 Low + Vehicle | 13 | −0.787 | 0.621 | 0.172 | 0.375 |
| CHS-131 High + Vehicle | 13 | −0.675 | 0.776 | 0.215 | 0.469 |
| Vehicle + Empagliflozin | 13 | −1.03 | 1.13 | 0.313 | 0.681 |
| CHS-131 High + | 14 | −1.02 | 0.994 | 0.266 | 0.574 |
| Empagliflozin | | | | | |
| Vehicle + Liraglutide | 14 | −1.28 | 0.818 | 0.219 | 0.472 |
| CHS-131 High + | 13 | −0.576 | 0.493 | 0.137 | 0.298 |
| Liraglutide | | | | | |
| Elafibranor + Vehicle | 13 | −1.27 | 0.884 | 0.245 | 0.534 |

### Effects of Treatments on Fasted Plasma Insulin

4 h fasted plasma insulin (pg/mL) at baseline. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 462 | 225 | 64.8 | 143 |
| NASH vehicle + vehicle | 13 | 769 | 221 | 61.2 | 133 |
| CHS-131 Low + Vehicle | 13 | 703 | 147 | 40.8 | 89 |
| CHS-131 High + Vehicle | 13 | 816 | 347 | 96.2 | 210 |
| Vehicle + Empagliflozin | 13 | 743 | 228 | 63.2 | 138 |
| CHS-131 High + | 14 | 626 | 183 | 48.8 | 106 |
| Empagliflozin | | | | | |
| Vehicle + Liraglutide | 14 | 644 | 218 | 58.3 | 126 |
| CHS-131 High + | 13 | 663 | 250 | 69.3 | 151 |
| Liraglutide | | | | | |
| Elafibranor + Vehicle | 13 | 644 | 121 | 33.5 | 72.9 |

4 h fasted plasma insulin (pg/mL) at week 12. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 372 | 143 | 41.2 | 90.7 |
| NASH vehicle + vehicle | 12 | 690 | 204 | 58.9 | 130 |
| CHS-131 Low + Vehicle | 13 | 485 | 114 | 31.5 | 68.7 |
| CHS-131 High + Vehicle | 13 | 402 | 112 | 31.1 | 67.7 |
| Vehicle + Empagliflozin | 13 | 673 | 285 | 79.1 | 172 |
| CHS-131 High + | 13 | 322 | 155 | 43.1 | 94 |
| Empagliflozin | | | | | |
| Vehicle + Liraglutide | 14 | 615 | 277 | 74 | 160 |
| CHS-131 High + | 13 | 426 | 115 | 32 | 69.6 |
| Liraglutide | | | | | |
| Elafibranor + Vehicle | 13 | 297 | 79 | 21.9 | 47.8 |

Delta 4 h fasted plasma insulin (pg/mL) week 12 vs baseline. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | −90.4 | 318 | 91.8 | 202 |
| NASH vehicle + vehicle | 12 | −92.3 | 262 | 75.7 | 167 |
| CHS-131 Low + Vehicle | 13 | −219 | 156 | 43.2 | 94.2 |
| CHS-131 High + Vehicle | 13 | −414 | 395 | 110 | 239 |
| Vehicle + Empagliflozin | 13 | −69.9 | 348 | 96.4 | 210 |
| CHS-131 High + | 13 | −320 | 211 | 58.6 | 128 |
| Empagliflozin | | | | | |
| Vehicle + Liraglutide | 14 | −28.8 | 341 | 91.1 | 197 |
| CHS-131 High + | 13 | −237 | 302 | 83.8 | 183 |
| Liraglutide | | | | | |
| Elafibranor + Vehicle | 13 | −347 | 122 | 33.9 | 73.9 |

### Effects of Treatments on Glucose Tolerance as Assessed by Oral Glucose Tolerance Test

Oral glucose tolerance test (OGTT) in week 7. Temporal course of blood glucose levels during 6 h fasted OGTT. Oral glucose bolus (2 g/kg) at t=0 min.

Area under the curve calculated from the temporal course from 0-180 min OGTT. Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 1.53e+03 | 119 | 34.4 | 75.8 |
| NASH vehicle + vehicle | 13 | 1.55e+03 | 189 | 52.3 | 114 |
| CHS-131 Low + Vehicle | 13 | 1.53e+03 | 171 | 47.4 | 103 |
| CHS-131 High + Vehicle | 13 | 1.66e+03 | 177 | 49.2 | 107 |
| Vehicle + Empagliflozin | 12 | 1.31e+03 | 109 | 31.5 | 69.3 |
| CHS-131 High + | 14 | 1.37e+03 | 139 | 37.3 | 80.5 |
| Empagliflozin | | | | | |
| Vehicle + Liraglutide | 14 | 1.36e+03 | 99.6 | 26.6 | 57.5 |
| CHS-131 High + | 13 | 1.41e+03 | 101 | 27.9 | 60.8 |
| Liraglutide | | | | | |
| Elafibranor + Vehicle | 13 | 1.35e+03 | 116 | 32.2 | 70.1 |

### Effects of Treatments on Insulin Sensitivity as Assessed by Intraperitoneal Insulin Tolerance Test

Intraperitoneal insulin tolerance test (IPITT) in week 10. Left: Temporal course of blood glucose levels during 6 h fasted IPITT. Intraperitoneal insulin bolus (0.5 U/kg) at t=0 min.

Area under the curve calculated from the temporal course from 0-180 min PITT Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 822 | 43.8 | 12.6 | 27.8 |
| NASH vehicle + vehicle | 12 | 868 | 101 | 29.1 | 64.1 |
| CHS-131 Low + Vehicle | 13 | 758 | 110 | 30.6 | 66.7 |
| CHS-131 High + Vehicle | 12 | 767 | 83.1 | 24 | 52.8 |
| Vehicle + Empagliflozin | 12 | 723 | 68.9 | 19.9 | 43.8 |
| CHS-131 High + | 14 | 756 | 65.5 | 17.5 | 37.8 |
| Empagliflozin | | | | | |
| Vehicle + Liraglutide | 14 | 728 | 67.4 | 18 | 38.9 |
| CHS-131 High + | 13 | 774 | 85.9 | 23.8 | 51.9 |
| Liraglutide | | | | | |
| Elafibranor + Vehicle | 13 | 757 | 123 | 34.1 | 74.4 |

### Effects of Treatments on Terminal Plasma Total Cholesterol

Plasma total cholesterol (TC) at termination (mmol/L). Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 2.74 | 0.263 | 0.076 | 0.167 |
| NASH vehicle + vehicle | 13 | 7.98 | 1.22 | 0.34 | 0.74 |
| CHS-131 Low + Vehicle | 13 | 6.45 | 0.855 | 0.237 | 0.517 |
| CHS-131 High + Vehicle | 13 | 6.38 | 1.37 | 0.38 | 0.828 |
| Vehicle + Empagliflozin | 13 | 7.62 | 1.38 | 0.383 | 0.834 |
| CHS-131 High + | 14 | 7.73 | 1.1 | 0.294 | 0.636 |
| Empagliflozin | | | | | |
| Vehicle + Liraglutide | 14 | 5.03 | 1.03 | 0.276 | 0.596 |
| CHS-131 High + | 13 | 4.71 | 0.479 | 0.133 | 0.29 |
| Liraglutide | | | | | |
| Elafibranor + Vehicle | 13 | 5.75 | 0.871 | 0.242 | 0.526 |

Plasma triglycerides (TG) at termination (mmol/L). Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 1.41 | 0.5 | 0.144 | 0.318 |
| NASH vehicle + vehicle | 13 | 0.883 | 0.248 | 0.0688 | 0.15 |
| CHS-131 Low + Vehicle | 13 | 0.715 | 0.224 | 0.0622 | 0.135 |
| CHS-131 High + Vehicle | 13 | 0.651 | 0.155 | 0.0431 | 0.094 |
| Vehicle + Empagliflozin | 13 | 0.738 | 0.122 | 0.0339 | 0.0739 |
| CHS-131 High + | 14 | 0.767 | 0.298 | 0.0797 | 0.172 |
| Empagliflozin | | | | | |
| Vehicle + Liraglutide | 14 | 0.649 | 0.171 | 0.0457 | 0.0988 |
| CHS-131 High + | 13 | 0.677 | 0.216 | 0.06 | 0.131 |
| Liraglutide | | | | | |
| Elafibranor + Vehicle | 12 | 0.525 | 0.202 | 0.0583 | 0.128 |

### Effects of Treatments on Terminal Plasma ALT and AST

Plasma alanine transaminase (ALT) at termination (U/L). Values expressed as mean of n=11-14+SEM.

| | | | | | 95% Conf. |
|---|---|---|---|---|---|
| Treatment | N | Average | Std. dev. | SEM | int. |
| CHOW vehicle + vehicle | 12 | 30.1 | 2.54 | 0.733 | 1.61 |
| NASH vehicle + vehicle | 11 | 222 | 121 | 36.4 | 81 |
| CHS-131 Low + Vehicle | 13 | 163 | 45.6 | 12.7 | 27.6 |
| CHS-131 High + Vehicle | 13 | 140 | 65.1 | 18 | 39.3 |
| Vehicle + Empagliflozin | 13 | 216 | 96.4 | 26.7 | 58.2 |
| CHS-131 High + | 14 | 155 | 64.2 | 17.2 | 37.1 |
| Empagliflozin | | | | | |
| Vehicle + Liraglutide | 14 | 47.8 | 27.9 | 7.45 | 16.1 |
| CHS-131 High + | 13 | 34.7 | 9.86 | 2.74 | 5.96 |
| Liraglutide | | | | | |
| Elafibranor + Vehicle | 12 | 144 | 95.2 | 27.5 | 60.5 |

Plasma aspartate transaminase (AST) at termination (U/L). Values expressed as mean of n=11-14+SEM.

| | | | | | 95% Conf. |
|---|---|---|---|---|---|
| Treatment | N | Average | Std. dev. | SEM | int. |
| CHOW vehicle + vehicle | 12 | 50.9 | 4.2 | 1.21 | 2.67 |
| NASH vehicle + vehicle | 13 | 234 | 108 | 30.1 | 65.6 |
| CHS-131 Low + Vehicle | 13 | 189 | 55.8 | 15.5 | 33.7 |
| CHS-131 High + Vehicle | 13 | 166 | 60.3 | 16.7 | 36.4 |
| Vehicle + Empagliflozin | 13 | 230 | 104 | 28.8 | 62.8 |
| CHS-131 High + | 14 | 193 | 67.5 | 18 | 39 |
| Empagliflozin | | | | | |
| Vehicle + Liraglutide | 14 | 97.2 | 36.9 | 9.85 | 21.3 |
| CHS-131 High + | 13 | 77.1 | 12.7 | 3.53 | 7.69 |
| Liraglutide | | | | | |
| Elafibranor + Vehicle | 13 | 205 | 110 | 30.4 | 66.3 |

### Effects of Treatment on Plasma Urea at Termination

Plasma urea at termination (mmol/L). Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 8.67 | 1.05 | 0.302 | 0.664 |
| NASH vehicle + vehicle | 13 | 7.48 | 0.5 | 0.139 | 0.302 |
| CHS-131 Low + Vehicle | 13 | 7.4 | 1.13 | 0.314 | 0.684 |
| CHS-131 High + Vehicle | 13 | 7.12 | 2.02 | 0.56 | 1.22 |
| Vehicle + Empagliflozin | 13 | 6.77 | 0.522 | 0.145 | 0.315 |
| CHS-131 High + | 14 | 7.13 | 1.09 | 0.292 | 0.632 |
| Empagliflozin | | | | | |
| Vehicle + Liraglutide | 14 | 7.86 | 1.16 | 0.311 | 0.671 |
| CHS-131 High + | 13 | 8.2 | 0.766 | 0.212 | 0.463 |
| Liraglutide | | | | | |
| Elafibranor + Vehicle | 13 | 8.01 | 1.32 | 0.366 | 0.798 |

### Effects of Treatments on Absolute and Relative Liver Weight

Absolute liver weight at termination (g). Values expressed as mean of n=12-14+SEM.

| | | | | | 95% Conf. |
|---|---|---|---|---|---|
| Treatment | N | Average | Std. dev. | SEM | int. |
| CHOW vehicle + vehicle | 12 | 1.36 | 0.19 | 0.055 | 0.121 |
| NASH vehicle + vehicle | 13 | 3.44 | 0.478 | 0.133 | 0.289 |
| CHS-131 Low + Vehicle | 13 | 3.21 | 0.62 | 0.172 | 0.374 |
| CHS-131 High + Vehicle | 13 | 3.14 | 0.626 | 0.174 | 0.378 |
| Vehicle + Empagliflozin | 13 | 3.37 | 0.542 | 0.15 | 0.327 |
| CHS-131 High + | 14 | 3.41 | 0.63 | 0.168 | 0.364 |
| Empagliflozin | | | | | |
| Vehicle + Liraglutide | 14 | 2.03 | 0.417 | 0.111 | 0.241 |
| CHS-131 High + | 13 | 2.22 | 0.247 | 0.0686 | 0.15 |
| Liraglutide | | | | | |
| Elafibranor + Vehicle | 13 | 4.2 | 0.962 | 0.267 | 0.581 |

Liver weight at termination in relative (% of body weight) values. Values expressed as mean of n=12-14+SEM.

| | | | | | |
|---|---|---|---|---|---|
| CHOW vehicle + vehicle | 12 | 4.49 | 0.635 | 0.183 | 0.403 |
| NASH vehicle + vehicle | 13 | 8.61 | 1.13 | 0.312 | 0.681 |
| CHS-131 Low + Vehicle | 13 | 8.4 | 1.54 | 0.428 | 0.933 |
| CHS-131 High + Vehicle | 13 | 8.15 | 1.13 | 0.312 | 0.68 |
| Vehicle + Empagliflozin | 13 | 8.48 | 1.09 | 0.303 | 0.66 |
| CHS-131 High + | 14 | 8.96 | 1.64 | 0.438 | 0.945 |
| Empagliflozin | | | | | |
| Vehicle + Liraglutide | 14 | 5.94 | 0.941 | 0.252 | 0.543 |
| CHS-131 High + | 13 | 6.39 | 0.655 | 0.182 | 0.396 |
| Liraglutide | | | | | |
| Elafibranor + Vehicle | 13 | 13.1 | 1.94 | 0.538 | 1.17 |

### Effects of Treatments on Relative and Total Liver Total Cholesterol at Termination

Relative (mg/g of liver) liver total cholesterol (TC) at termination. Values expressed as mean of n=12-14+SEM.

| | | | | | 95% |
|---|---|---|---|---|---|
| | | | Std. | | Conf. |
| Treatment | N | Average | dev. | SEM | int. |
| CHOW vehicle + vehicle | 12 | 3.95 | 0.359 | 0.104 | 0.228 |
| NASH vehicle + vehicle | 13 | 10.5 | 3.2 | 0.889 | 1.94 |
| CHS-131 Low + Vehicle | 13 | 12.4 | 2.63 | 0.73 | 1.59 |
| CHS-131 High + Vehicle | 13 | 12.3 | 2.69 | 0.747 | 1.63 |
| Vehicle + Empagliflozin | 13 | 12.6 | 3.47 | 0.964 | 2.1 |
| CHS-131 High + Empagliflozin | 14 | 12 | 3.53 | 0.943 | 2.04 |
| Vehicle + Liraglutide | 14 | 8.73 | 2.07 | 0.554 | 1.2 |
| CHS-131 High + Liraglutide | 13 | 8.77 | 1.99 | 0.552 | 1.2 |
| Elafibranor + Vehicle | 13 | 8.87 | 1.54 | 0.427 | 0.93 |

Total (mg/liver) liver total cholesterol (TC) at termination. Values expressed as mean of n=1214+SEM.

| | |
|---|---|
| | 95% |
| Std. | Conf. |

| Treatment | N | Average | dev. | SEM | int. |
|---|---|---|---|---|---|
| CHOW vehicle + vehicle | 12 | 5.39 | 0.911 | 0.263 | 0.579 |
| NASH vehicle + vehicle | 13 | 36.5 | 13.4 | 3.71 | 8.09 |
| CHS-131 Low + Vehicle | 13 | 39.6 | 10.1 | 2.79 | 6.08 |
| CHS-131 High + Vehicle | 13 | 39.2 | 13.1 | 3.64 | 7.93 |
| Vehicle + Empagliflozin | 13 | 43.2 | 16.6 | 4.59 | 10 |
| CHS-131 High + Empagliflozin | 14 | 40.2 | 11.8 | 3.16 | 6.82 |
| Vehicle + Liraglutide | 14 | 17.5 | 4.46 | 1.19 | 2.58 |
| CHS-131 High + Liraglutide | 13 | 19.8 | 6.16 | 1.71 | 3.72 |
| Elafibranor + Vehicle | 13 | 38.3 | 16.3 | 4.53 | 9.88 |

### Effects of Treatments on Relative and Total Terminal Liver Triglycerides

Relative (mg/g of liver) liver triglycerides (TG) at termination. Values expressed as mean of n=12-14+SEM.

| | |
|---|---|
| | 95% |
| Std. | Conf. |

| Treatment | N | Average | dev. | SEM | int. |
|---|---|---|---|---|---|
| CHOW vehicle + vehicle | 12 | 9.42 | 1.73 | 0.5 | 1.1 |
| NASH vehicle + vehicle | 13 | 89.1 | 40.4 | 11.2 | 24.4 |
| CHS-131 Low + Vehicle | 13 | 96.7 | 27.6 | 7.66 | 16.7 |
| CHS-131 High + Vehicle | 13 | 92.9 | 30.5 | 8.46 | 18.4 |
| Vehicle + Empagliflozin | 13 | 108 | 30.4 | 8.44 | 18.4 |
| CHS-131 High + Empagliflozin | 14 | 95.4 | 26.5 | 7.09 | 15.3 |
| Vehicle + Liraglutide | 14 | 57 | 17.4 | 4.66 | 10.1 |
| CHS-131 High + Liraglutide | 13 | 50.2 | 24.3 | 6.75 | 14.7 |
| Elafibranor + Vehicle | 13 | 45.2 | 17.8 | 4.94 | 10.8 |

Total (mg/liver) liver triglycerides (TG) at termination. Values expressed as mean of n=12-14+SEM.

| | |
|---|---|
| | 95% |
| Std. | Conf. |

| Treatment | N | Average | dev. | SEM | int. |
|---|---|---|---|---|---|
| CHOW vehicle + vehicle | 12 | 12.9 | 3.17 | 0.915 | 2.01 |
| NASH vehicle + vehicle | 13 | 313 | 161 | 44.7 | 97.4 |
| CHS-131 Low + Vehicle | 13 | 306 | 88.1 | 24.4 | 53.3 |
| CHS-131 High + Vehicle | 13 | 300 | 126 | 35 | 76.2 |
| Vehicle + Empagliflozin | 13 | 368 | 137 | 37.9 | 82.7 |
| CHS-131 High + Empagliflozin | 14 | 318 | 86.7 | 23.2 | 50 |
| Vehicle + Liraglutide | 14 | 115 | 40.9 | 10.9 | 23.6 |
| CHS-131 High + Liraglutide | 13 | 115 | 68.5 | 19 | 41.4 |
| Elafibranor + Vehicle | 13 | 196 | 106 | 29.4 | 64.1 |

### Effects of Treatments on Relative Liver Hydroxyproline Levels at Termination

Relative liver hydroxyproline (HP) (μg/mg of liver) at termination. Values expressed as mean of n=12-14+SEM.

| | |
|---|---|
| | 95% |
| Std. | Conf. |

| Treatment | N | Average | dev. | SEM | int. |
|---|---|---|---|---|---|
| CHOW vehicle + vehicle | 12 | 0.0193 | 0.00356 | 0.00103 | 0.00226 |
| NASH vehicle + vehicle | 13 | 0.0873 | 0.0292 | 0.00809 | 0.0176 |
| CHS-131 Low + Vehicle | 13 | 0.0631 | 0.0154 | 0.00427 | 0.0093 |
| CHS-131 High + Vehicle | 12 | 0.0802 | 0.0219 | 0.00632 | 0.0139 |
| Vehicle + Empagliflozin | 13 | 0.0747 | 0.0201 | 0.00558 | 0.0122 |
| CHS-131 High + | 14 | 0.075 | 0.0283 | 0.00756 | 0.0163 |
| Empagliflozin | | | | | |
| Vehicle + Liraglutide | 14 | 0.063 | 0.0252 | 0.00673 | 0.0145 |
| CHS-131 High + | 13 | 0.069 | 0.0165 | 0.00458 | 0.00998 |
| Liraglutide | | | | | |
| Elafibranor + Vehicle | 13 | 0.0389 | 0.0141 | 0.0039 | 0.00849 |

Total liver hydroxyproline (HP) (μg/liver) at termination. Values expressed as mean of n=12-14+SEM.

| | |
|---|---|
| | 95% |
| Std. | Conf. |

| Treatment | N | Average | dev. | SEM | int. |
|---|---|---|---|---|---|
| CHOW vehicle + vehicle | 12 | 25.9 | 4.35 | 1.26 | 2.76 |
| NASH vehicle + vehicle | 13 | 300 | 106 | 29.3 | 63.8 |
| CHS-131 Low + Vehicle | 13 | 205 | 70.7 | 19.6 | 42.7 |
| CHS-131 High + Vehicle | 12 | 250 | 94.5 | 27.3 | 60.1 |
| Vehicle + Empagliflozin | 13 | 233 | 100 | 27.8 | 60.6 |
| CHS-131 High + Empagliflozin | 14 | 261 | 136 | 36.5 | 78.7 |
| Vehicle + Liraglutide | 14 | 131 | 72.6 | 19.4 | 41.9 |
| CHS-131 High + Liraglutide | 13 | 153 | 39.5 | 10.9 | 23.8 |
| Elafibranor + Vehicle | 13 | 164 | 75.5 | 20.9 | 45.6 |

### Effects of Treatments on Delta NAFLD Activity Score

Delta change in NAFLD activity score from pre-to post biopsy. Values expressed as mean of n=12-14+SEM.

| Treatment | N | Average |
|---|---|---|
| CHOW vehicle + vehicle | 12 | −0.08 |
| NASH vehicle + vehicle | 13 | 0.15 |
| CHS-131 Low + Vehicle | 13 | −0.23 |
| CHS-131 High + Vehicle | 12 | −0.77 |
| Vehicle + Empagliflozin | 13 | −0.31 |
| CHS-131 High + Empagliflozin | 14 | −0.21 |
| Vehicle + Liraglutide | 14 | −1.36 |
| CHS-131 High + Liraglutide | 13 | −1.85 |
| Elafibranor + Vehicle | 13 | −2.00 |

Administration of CHS-131 reduced NAS compared to NASH vehicle+vehicle.

### Effects of Treatments on Relative and Total Liver Steatosis

Terminal liver steatosis (i.e. liver lipid) was quantified by morphometry. Relative liver steatosis is denoted in % fractional area. Values expressed as mean of n=12-14+SEM.

| | |
|---|---|
| | 95% |
| Std. | Conf. |

| Treatment | N | Average | dev. | SEM | int. |
|---|---|---|---|---|---|
| CHOW vehicle + vehicle | 12 | 1.13 | 0.185 | 0.0535 | 0.118 |
| CHS-131 High + Empagliflozin | 14 | 25.2 | 5.78 | 1.54 | 3.33 |
| CHS-131 High + Liraglutide | 13 | 11.6 | 4.9 | 1.36 | 2.96 |
| CHS-131 High + Vehicle | 13 | 23.1 | 8.62 | 2.39 | 5.21 |
| CHS-131 Low + Vehicle | 13 | 26.4 | 6.23 | 1.73 | 3.76 |
| Elafibranor + Vehicle | 13 | 7.21 | 3.87 | 1.07 | 2.34 |
| NASH vehicle + vehicle | 13 | 26 | 6.09 | 1.69 | 3.68 |
| Vehicle + Empagliflozin | 13 | 27.2 | 3.55 | 0.986 | 2.15 |
| Vehicle + Liraglutide | 14 | 13.1 | 3.9 | 1.04 | 2.25 |

Total liver steatosis is denoted in milligrams (% fractional area multiplied with liver weight). Values expressed as mean of n=12-14+SEM.

| | | |
|---|---|---|
| | | 95% |
| Aver- | Std. | Conf. |

| Treatment | N | age | dev. | SEM | int. |
|---|---|---|---|---|---|
| CHOW vehicle + vehicle | 12 | 15.2 | 1.93 | 0.556 | 1.22 |
| CHS-131 High + Empagliflozin | 14 | 854 | 247 | 66 | 142 |
| CHS-131 High + Liraglutide | 13 | 265 | 135 | 37.3 | 81.3 |
| CHS-131 High + Vehicle | 13 | 758 | 344 | 95.5 | 208 |
| CHS-131 Low + Vehicle | 13 | 826 | 183 | 50.7 | 110 |
| Elafibranor + Vehicle | 13 | 322 | 229 | 63.4 | 138 |
| NASH vehicle + vehicle | 13 | 897 | 243 | 67.5 | 147 |
| Vehicle + Empagliflozin | 13 | 923 | 211 | 58.6 | 128 |
| Vehicle + Liraglutide | 14 | 278 | 122 | 32.6 | 70.5 |

### Effects of Treatments on Relative and Total Liver Col1a1 Content

Terminal liver Collagen 1A1 (Col1a1) was quantified by morphometry. Relative Terminal liver Col1a1 (% fractional area) is expressed as mean of n=12-14+SEM.

| | |
|---|---|
| | 95% |
| Std. | Conf. |

| Treatment | N | Average | dev. | SEM | int. |
|---|---|---|---|---|---|
| CHOW vehicle + vehicle | 12 | 1.83 | 0.304 | 0.0878 | 0.193 |
| CHS-131 High + Empagliflozin | 14 | 6.59 | 2.04 | 0.545 | 1.18 |
| CHS-131 High + Liraglutide | 13 | 7.14 | 2.27 | 0.63 | 1.37 |
| CHS-131 High + Vehicle | 13 | 6.77 | 2.08 | 0.576 | 1.25 |
| CHS-131 Low + Vehicle | 13 | 6.64 | 2.08 | 0.576 | 1.26 |
| Elafibranor + Vehicle | 13 | 6.27 | 1.76 | 0.488 | 1.06 |
| NASH vehicle + vehicle | 13 | 8.01 | 3.23 | 0.895 | 1.95 |
| Vehicle + Empagliflozin | 13 | 7.31 | 2.01 | 0.558 | 1.22 |
| Vehicle + Liraglutide | 14 | 5.51 | 2.35 | 0.627 | 1.35 |

Total terminal Col1a1 values are in milligrams (% fractional area multiplied with liver weight) and expressed as mean of n=12-14+SEM.

| | |
|---|---|
| | 95% |
| Std. | Conf. |

| Treatment | N | Average | dev. | SEM | int. |
|---|---|---|---|---|---|
| CHOW vehicle + vehicle | 12 | 24.8 | 4.2 | 1.21 | 2.67 |
| CHS-131 High + Empagliflozin | 14 | 230 | 95.3 | 25.5 | 55 |
| CHS-131 High + Liraglutide | 13 | 157 | 45.7 | 12.7 | 27.6 |
| CHS-131 High + Vehicle | 13 | 220 | 90 | 25 | 54.4 |
| CHS-131 Low + Vehicle | 13 | 213 | 72 | 20 | 43.5 |
| Elafibranor + Vehicle | 13 | 270 | 136 | 37.7 | 82.2 |
| NASH vehicle + vehicle | 13 | 274 | 108 | 30.1 | 65.5 |
| Vehicle + Empagliflozin | 13 | 250 | 84.1 | 23.3 | 50.8 |
| Vehicle + Liraglutide | 14 | 117 | 77.6 | 20.7 | 44.8 |

### Effects of Treatments on Relative and Total Liver α-SMA Levels at Termination

Terminal liver α-SMA was quantified by morphometry. Relative terminal liver α-SMA (% fractional area) is expressed as mean of n=12-14+SEM.

| | |
|---|---|
| | 95% |
| Std. | Conf. |

| Treatment | N | Average | dev. | SEM | int. |
|---|---|---|---|---|---|
| CHOW vehicle + vehicle | 12 | 0.47 | 0.17 | 0.0492 | 0.108 |
| CHS-131 High + Empagliflozin | 14 | 3.27 | 1.39 | 0.37 | 0.8 |
| CHS-131 High + Liraglutide | 13 | 1.52 | 0.368 | 0.102 | 0.222 |
| CHS-131 High + Vehicle | 13 | 3.07 | 1.22 | 0.34 | 0.74 |
| CHS-131 Low + Vehicle | 13 | 3.49 | 1.29 | 0.357 | 0.777 |
| Elafibranor + Vehicle | 13 | 2.56 | 1 | 0.278 | 0.606 |
| NASH vehicle + vehicle | 13 | 3.69 | 0.917 | 0.254 | 0.554 |
| Vehicle + Empagliflozin | 13 | 3.8 | 0.75 | 0.208 | 0.453 |
| Vehicle + Liraglutide | 14 | 1.53 | 0.558 | 0.149 | 0.322 |

Total terminal α-SMA values are in milligrams (% fractional area multiplied with liver weight) and expressed as mean of n=12-14+SEM.

| | |
|---|---|
| | 95% |
| Std. | Conf. |

| Treatment | N | Average | dev. | SEM | int. |
|---|---|---|---|---|---|
| CHOW vehicle + vehicle | 12 | 6.38 | 2.22 | 0.642 | 1.41 |
| CHS-131 High + Empagliflozin | 14 | 113 | 56.5 | 15.1 | 32.6 |
| CHS-131 High + Liraglutide | 13 | 33.9 | 9.54 | 2.65 | 5.76 |
| CHS-131 High + Vehicle | 13 | 102 | 49.7 | 13.8 | 30 |
| CHS-131 Low + Vehicle | 13 | 110 | 42.1 | 11.7 | 25.4 |
| Elafibranor + Vehicle | 13 | 114 | 78 | 21.6 | 47.1 |
| NASH vehicle + vehicle | 13 | 128 | 40.2 | 11.1 | 24.3 |
| Vehicle + Empagliflozin | 13 | 130 | 38.9 | 10.8 | 23.5 |
| Vehicle + Liraglutide | 14 | 32.5 | 17.8 | 4.76 | 10.3 |

### Effects of Treatments on Relative and Total Liver Galectin-3 Levels at Termination

Terminal liver Galectin-3 was quantified by morphometry. Relative terminal liver Galectin-3 (% fractional area) is expressed as mean of n=12-14+SEM.

| | |
|---|---|
| | 95% |
| Std. | Conf. |

| Treatment | N | Average | dev. | SEM | int. |
|---|---|---|---|---|---|
| CHOW vehicle + vehicle | 12 | 0.557 | 0.13 | 0.0376 | 0.0828 |
| CHS-131 High + Empagliflozin | 14 | 3.05 | 1 | 0.267 | 0.578 |
| CHS-131 High + Liraglutide | 13 | 1.77 | 0.257 | 0.0713 | 0.155 |
| CHS-131 High + Vehicle | 13 | 2.79 | 0.892 | 0.247 | 0.539 |
| CHS-131 Low + Vehicle | 13 | 2.95 | 0.719 | 0.199 | 0.434 |
| Elafibranor + Vehicle | 13 | 2.43 | 0.56 | 0.155 | 0.338 |
| NASH vehicle + vehicle | 13 | 3.17 | 0.737 | 0.205 | 0.446 |
| Vehicle + Empagliflozin | 13 | 2.9 | 0.652 | 0.181 | 0.394 |
| Vehicle + Liraglutide | 14 | 1.68 | 0.385 | 0.103 | 0.222 |

Total terminal Galectin-3 values are in milligrams (% fractional area multiplied with liver weight) and expressed as mean of n=12-14+SEM.

| | |
|---|---|
| | 95% |
| Std. | Conf. |

| Treatment | N | Average | dev. | SEM | int. |
|---|---|---|---|---|---|
| CHOW vehicle + vehicle | 12 | 7.49 | 1.65 | 0.476 | 1.05 |
| CHS-131 High + Empagliflozin | 14 | 105 | 41.6 | 11.1 | 24 |
| CHS-131 High + Liraglutide | 13 | 39.2 | 6.7 | 1.86 | 4.05 |
| CHS-131 High + Vehicle | 13 | 90.6 | 40.5 | 11.2 | 24.5 |
| CHS-131 Low + Vehicle | 13 | 93.8 | 24.5 | 6.79 | 14.8 |
| Elafibranor + Vehicle | 13 | 104 | 42.3 | 11.7 | 25.6 |
| NASH vehicle + vehicle | 13 | 110 | 36.1 | 10 | 21.8 |
| Vehicle + Empagliflozin | 13 | 99.9 | 34.9 | 9.69 | 21.1 |
| Vehicle + Liraglutide | 14 | 34.5 | 11.8 | 3.17 | 6.84 |

### Summary of Data for CHS-131 Administered Alone or in Combination

CHS-131 alone caused significant improvement in insulin sensitization and the NAFLD activity score. Additionally, positive trends for improvement in markers of fibrosis (hydroxyproline and α-SMA) were observed. Moreover, CHS-131 reduced fat tissue mass, plasma ALT, plasma insulin, and plasma TC.

Liraglutide alone or in combination with CHS-131 significantly improved the NAFLD activity score accompanied by a reduction in steatosis and lobular inflammation. The combination of liraglutide plus CHS-131 improved the NASH activity score, especially steatosis, in comparison to either alone.

Empagliflozin alone or in combination with CHS-131 showed a positive trend for improvement in the NAFLD activity score and significant reduction of markers of fibrosis.

CHS-131 either alone or in combination with liraglutide showed the most promise for improving the NAFLD activity score in the NASH population.

Selected effects of CHS-131 are summarized below in various groups: CHS-131 monotherapy compared to placebo (DIO mice administered vehicle only); CHS-131 in combination with liraglutide compared to liraglutide monotherapy; CHS-131 in combination with empagliflozin compared to empagliflozin monotherapy; CHS-131 in combination with empagliflozin compared to placebo. Significance is considered when p <0.05 one tailed and trend if >0.05 one tailed.

### Selected Effects of CHS-131 as a Monotherapy

When compared to DIO mice (high fat diet) administered vehicle only (e.g. placebo), CHS-131 as a monotherapy provided (over the 11 week study), for example:
CHS-131 reduced significantly fat mass.
CHS-131 reduced relative fat tissue mass.
CHS-131 reduced % fat mass as % of baseline body weight by 20% when compared to vehicle alone.
Free water mass remained stable when CHS-131 is administered. Thus, reductions observed in body weight does not result from reducing water mass.
No difference was observed in relative liver weight.
No difference was observed in hemodilution, assessed by urea, when CHS-131 was administered.
Fasting plasma insulin decreased significantly in response to CHS-131 in a dose response manner by 50%.
Insulin resistance decreased significantly in response to CHS-131, but not in a dose response manner, by 15%.
Plasma cholesterol decreased significantly in response to CHS-131, but not in a dose response manner, by ˜12%.
Plasma alanine aminotransferase (ALT) decreased significantly in response to CHS-131 in a dose response manner by 30%.
Liver hydroxyproline level at termination (marker of fibrosis) improved with CHS-131 significantly by 30%.

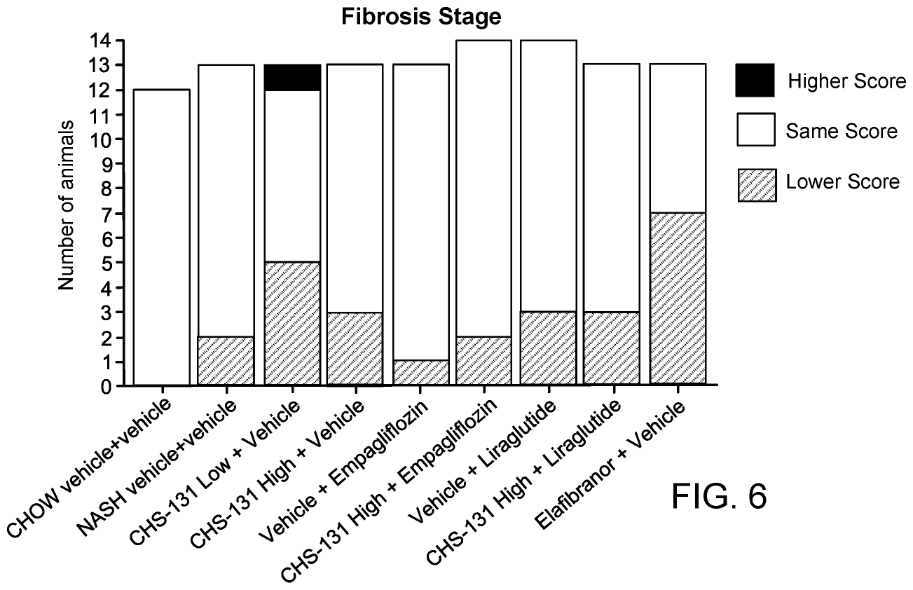

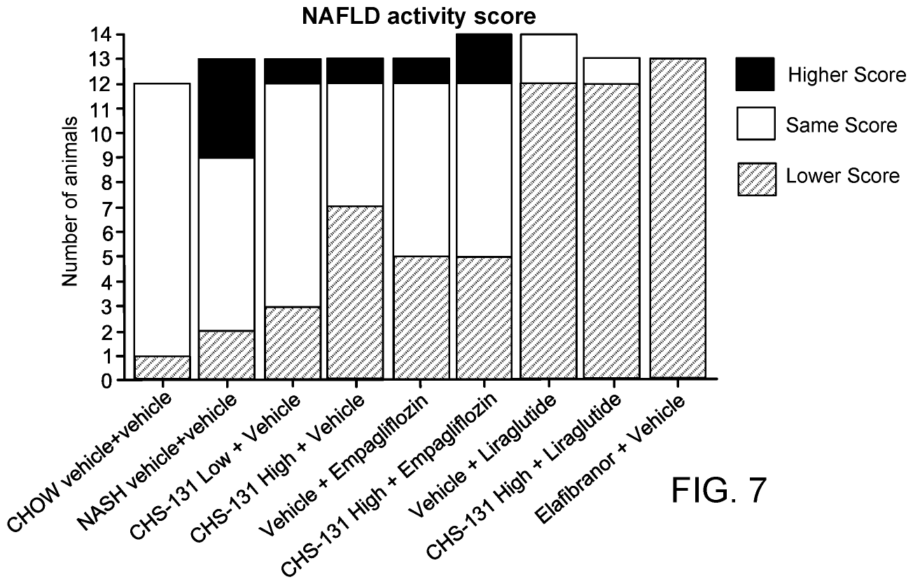

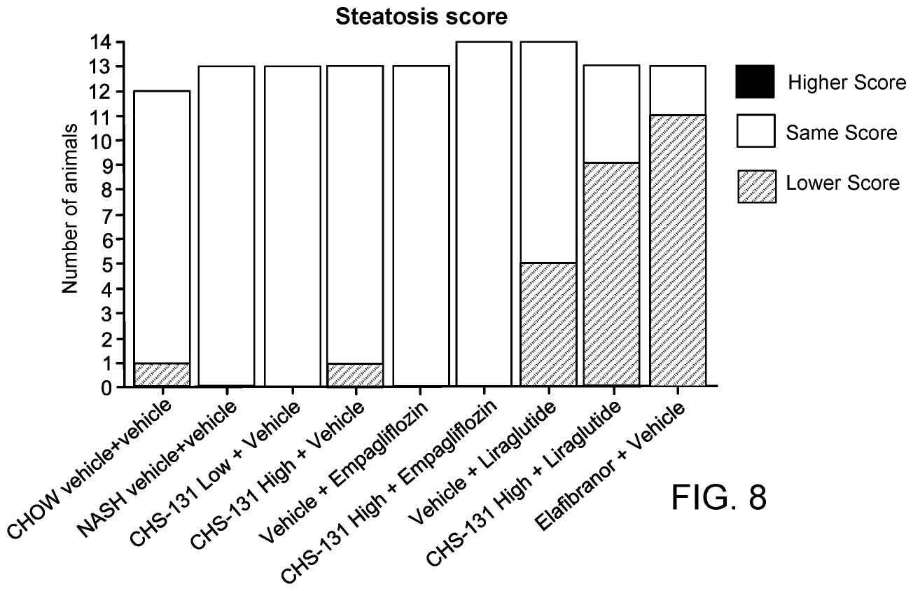

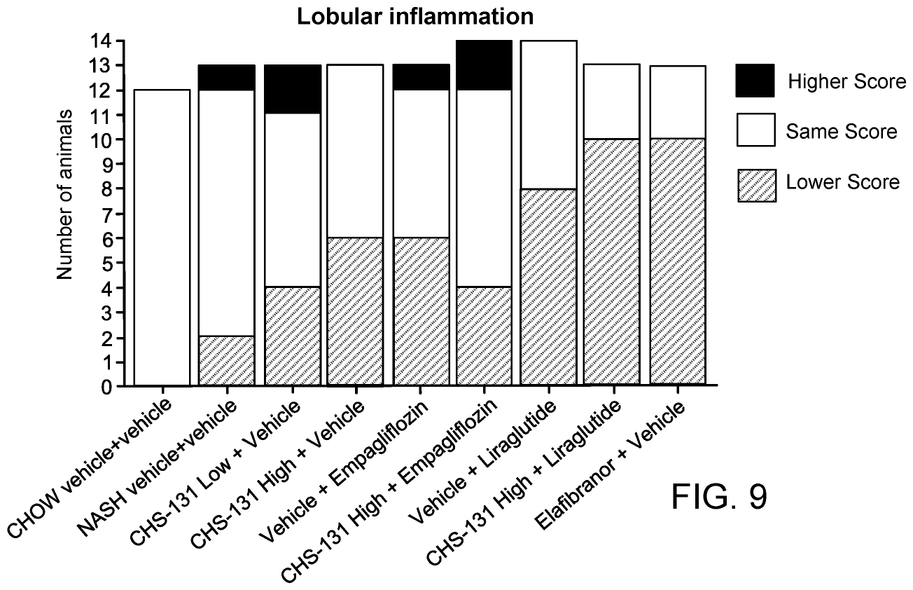

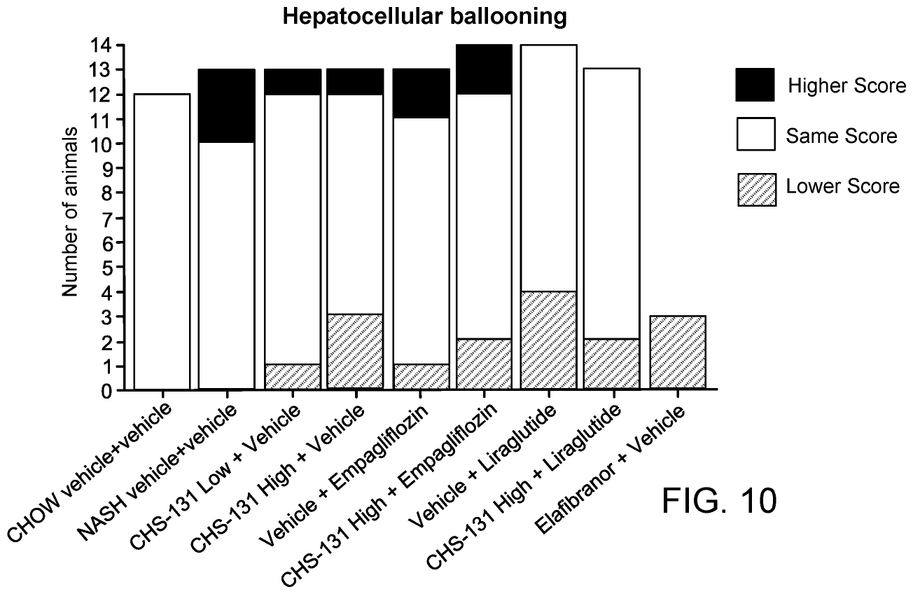

In addition:
CHS-131 tends to reduce body weight.
Fibrosis stage tends to improve with CHS-131 (p=0.07) in 5/13 mice.
NAFLD score improved significantly in 7/13 mice. See FIG. 6-FIG. 10.
Average NAFLD activity score (NAS) post-baseline improved significantly by more than 1 unit on average.
Lobular inflammation tends to improve by 50% non-significantly (p=0.07).
Steatosis and ballooning tend to improve non-significantly by approximately 30% and 10% respectively.
Terminal Collagen 1a1 (marker of fibrosis) tends to decrease by 15% when compared to vehicle alone.
Terminal α-SMA (marker of fibrosis) tends to decrease by 15% when compared to vehicle alone.
   Selected Effects of CHS-131 in Combination with Liraglutide

When compared to DIO mice administered liraglutide only, CHS-131 and liraglutide in combination provided (over the 11 week study), for example:
No difference in free water mass—remains stable with or without CHS-131
Fasting plasma insulin decreased significantly in response to CHS-131 by 25%
Steatosis and ballooning improved significantly in response to adding CHS-131 to Lira by approximately 50% in both cases.

In addition:
CHS-131 and liraglutide in combination tends to decrease fat mass at week 11 (end of study—sacrifice).
Plasma alanine aminotransferase (ALT) tends to decrease significantly in response to adding CHS-131.
Average NAFLD activity score post-baseline tends to improve by approximately 15% or 0.15 units on average.
Lobular inflammation tends to improve by 20% non-significantly.
Terminal liver lipids tend to decrease.
   Selected Effects of CHS-131 in Combination with Empagliflozin

When compared to DIO mice administered empagliflozin only, CHS-131 and empagliflozin in combination provided (over the 11 weeks study), for example:
Decreased fat mass in.
Reduced relative fat tissue mass.
Reduced % fat mass as % of baseline Body weight by 10%.
No difference in free water mass—remains stable with or without CHS-131.
Fasting plasma insulin decreased significantly in response to CHS-131 by 50%.
No difference in relative liver weight.
No difference in hemodilution assessed by urea—pertinent negative which indicates CHS-131 does not induce edema.

In addition:
Plasma alanine aminotransferase (ALT) tends to decrease significantly in response to adding CHS-131 by 20%
Liver lipids tend to decrease.
Terminal α-SMA (marker of fibrosis) tends to decrease by 5% when compared to vehicle alone.
Terminal Collagen 1a1 (marker of fibrosis) tends to decrease by 5% when compared to vehicle alone.

When compared to DIO mice administered placebo, CHS-131 and empagliflozin in combination provided (over the 11 week study), for example:
Decreased fat mass in week 11 (end of study—sacrifice) By approx 25%
Fasting plasma insulin decreases significantly by 350 pg/mL.

In addition:
Plasma alanine aminotransferase (ALT) tends to decrease significantly in response to adding empa+CHS-131 by about 28%.
Average NAFLD activity score post-baseline tends to improve by approximately from 2/13 in DIO to 5/13 animals with combo.
Terminal Collagen 1a1 (marker of fibrosis) tends to decrease by 8% in absolute values and about 15% when expressed at total fractional area.

### Example 3

This study assesses the effects of treatment with CHS-131 (Compound of Formula (I)), alone and in combination with other therapeutic agents, to treat NASH. Metabolic parameters, hepatic pathology, and NAFLD Activity Score including fibrosis stage are evaluated in ob/ob mice. In addition to the description below, this study may include sample collection, testing, measurement, and evaluation (e.g. histology, biochemical, gene expression, genetic), and analysis as described in the examples above.

ob/ob mice are homozygous for a spontaneous Lep^{ob} point mutation in the gene encoding leptin and are consistently fibrosis prone when cholesterol (2%) and trans-fatty acids (45% of total fat amount) are added to a high-caloric diet. These mice will develop steatohepatitis and fibrosis within a shorter timeframe (≤12 weeks) compared with wild-type C57BL/6 mice fed the same diet (≥26 weeks). See, e.g., Kristiansen, et al., World J. Hepatol., Vol. 8, pp. 673-684 (2016). The ob/ob mice also display a more significant insulin resistant and NASH phenotype than the high-caloric diet, well suited for evaluating potential anti-NASH therapeutics. Exemplary protocols for NASH mouse models are found in Tølbøl, et al., World J Gastroenterol. 2018 Jan. 14; 24(2):179-194, Roth, et al., Sci Rep. 2019 Jun. 21; 9(1):9046, and Boland, et al., World J Gastroenterol. 2019 Sep. 7; 25(33):4904-4920, which are hereby incorporated by reference in their entirety.

In this study, ob/ob-NASH mice are divided into 4 ob/ob-NASH groups (e.g. n=14 for each group) with dosing for 12 weeks (PO, QD). Male B6.V-Lep^{ob}/JRj mice are fed 40% HFD, 20% fructose, 2% Cholesterol (GAN) diet for 12+ weeks prior to study start.

All mice entering the experiment are pre-biopsied at week −4 and stratified based on liver biopsy with only animals with fibrosis stage ≥1, inflammation score ≥2 and steatosis score ≥2 being included in the study. Animals are randomized into groups based on fibrosis stage as measured by picosirius red (PSR) staining. Total of 12 weeks of PO, QD dosing. The four groups are as follows: 1) Vehicle, 2) CHS-131, 30 mg/kg, 3) Dapagliflozin, 1 mg/kg, 4) CHS-131, 30 mg/kg+Dapagliflozin, 1 mg/kg.

Body weight is measured daily during the study period. Four hour fasting plasma glucose and HbA1c are measured at baseline, week 6, and week 12. Fasting plasma insulin and terminal plasma ALT/AST/GGT/ and lipids are also measured at baseline and at week 12.

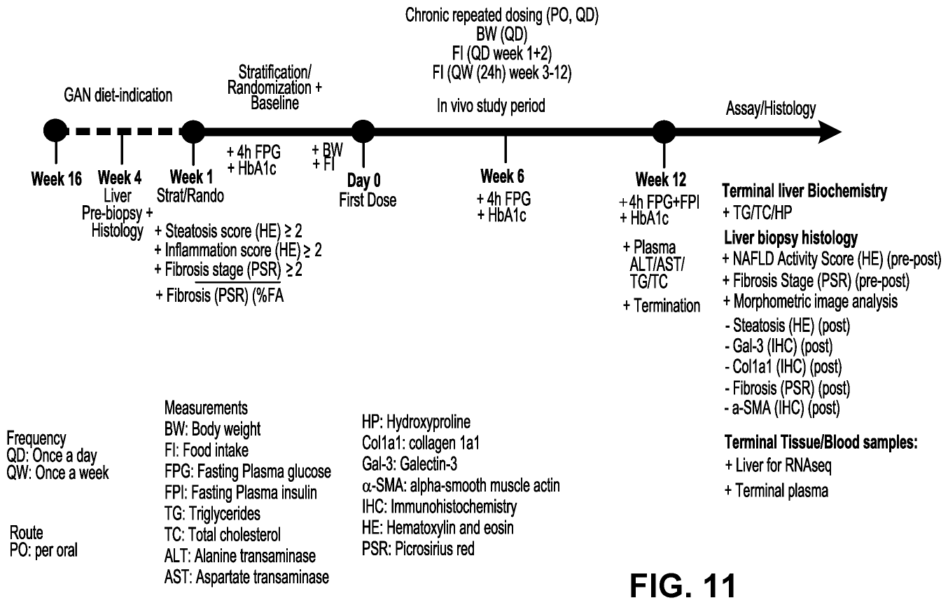

Terminal liver removal, weighing, and sampling at week 12 includes 1) FFPE (histology), 2) biochemical analysis, and 3) RNAseq analysis. Liver biopsy histology includes determination of 1) pre-to-post NAFLD Activity Score including Fibrosis Stage, 2) post steatosis (HE), 3) post Galectin-3 (IHC), an inflammation biomarker; other marker of an inflammatory response such as eicosanoids, hydroxyeicosatetraenoic acids (HETEs) and prostaglandins, are also measured, 4) post-fibrosis (PSR), 5) fibrosis biomarkers, including post Col1a1 (IHC), 6) post α-SMA (IHC). Additional fibrosis biomarkers are optionally measured including Pro-C3, C3M, Pro-C6 and C6M (Nordic Biosciences, Herlev, Denmark) which may characterize an observed anti-fibrotic effect. Liver TG/TC/IP content is also determined. Total adiponectin is measured at baseline and end-of-study. A study outline is shown in FIG. 11.

What is claimed is:

1. A method of treating non-alcoholic fatty liver disease (NAFLD) in a subject in need thereof comprising administering to the subject
(a) the compound of Formula (I), 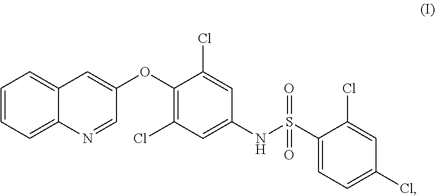 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof, wherein the amounts of (a) and (b) together are effective in treating NAFLD.

2. A method of treating a subject, the method comprising:
selecting a subject having non-alcoholic fatty liver disease (NAFLD); and
administering
(a) the compound of Formula (I), 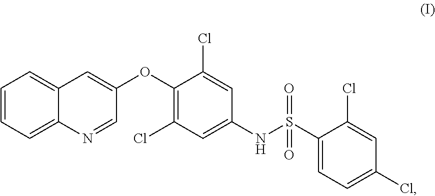 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof, to the selected subject,
wherein the amounts of (a) and (b) together are effective in treating NAFLD.

3. A method of treating a subject, the method comprising:
identifying a subject having non-alcoholic fatty liver disease (NAFLD); and
administering
(a) the compound of Formula (I), 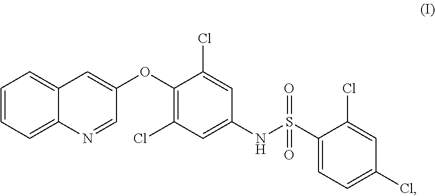 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof, to the selected subject,
wherein the amounts of (a) and (b) together are effective in treating NAFLD.

4. A method of treating non-alcoholic fatty liver disease (NAFLD) in a subject in need thereof comprising administering to the subject
(a) a therapeutically effective amount of the compound of Formula (I), 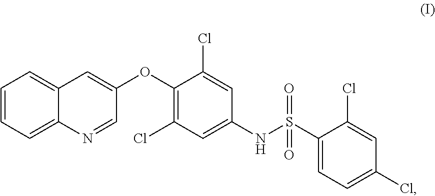 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) a therapeutically effective amount of an SGLT-2 inhibitor, or a pharmaceutically acceptable salt or solvate thereof.

5. A method of treating a subject, the method comprising:
selecting a subject having non-alcoholic fatty liver disease (NAFLD); and
administering
(a) a therapeutically effective amount of the compound of Formula (I), 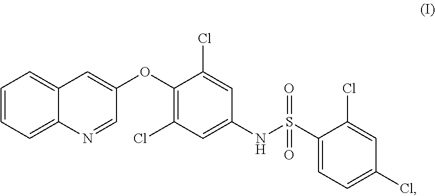 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) a therapeutically effective amount of an SGLT-2 inhibitor, or a pharmaceutically acceptable salt or solvate thereof, to the selected subject.

6. A method of treating fibrosis in a subject in need thereof comprising administering to the subject
(a) the compound of Formula (I), 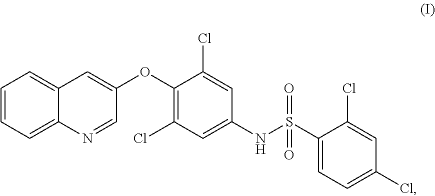 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof, wherein the amounts of (a) and (b) together are effective in treating fibrosis.

7. A method of treating fibrosis in a subject in need thereof comprising administering to the subject
(a) a therapeutically effective amount of the compound of Formula (I), 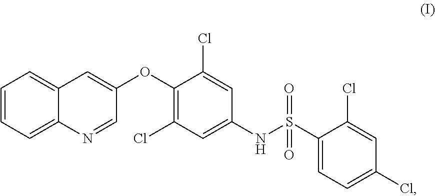 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) a therapeutically effective amount of an SGLT-2 inhibitor, or a pharmaceutically acceptable salt or solvate thereof.

8. The method of claims 6 or 7, wherein the fibrosis is cirrhosis.

9. The method of any one of claims 6 to 8, wherein the fibrosis is associated with NAFLD.

10. The method of any one of claims 6 to 9, wherein the fibrosis is caused by NAFLD.

11. The method of any one of claims 1 to 10, wherein the NAFLD is NASH.

12. The method of any one of claims 1 to 11, wherein the SGLT-2 inhibitor is selected from the group consisting of: empagliflozin, canagliflozin, dapagliflozin, ertugliflozin, ipragliflozin, luseogliflozin, remogliflozin etabonate, serfliflozin etabonate, sotagliflozin, tofogliflozin, or a combination of two or more thereof.

13. The method of any one of claims 1 to 12, wherein the SGLT-2 inhibitor is empagliflozin or dapagliflozin propylene glycol hydrate.

14. The method of any one of claims 1 to 13, wherein (a) and (b) are administered concurrently.

15. The method of any one of claims 1 to 14, wherein (a) and (b) are administered sequentially in either order.

16. The method of any one of claims 1 to 15, wherein the SGLT-2 inhibitor, or a pharmaceutically acceptable salt or solvate thereof, is administered at a dose from about 1 to about 350 mg.

17. The method of any one of claims 1 to 16, wherein the SGLT-2 inhibitor, or a pharmaceutically acceptable salt or solvate thereof, is administered at a dose from about 85 to about 325 mg.

18. The method of any one of claims 1 to 17, wherein the SGLT-2 inhibitor, or a pharmaceutically acceptable salt or solvate thereof, is administered at a dose from about 5 to about 15 mg.

19. The method of any one of claims 1 to 18, wherein the SGLT-2 inhibitor, or a pharmaceutically acceptable salt or solvate thereof, is administered at a dose of about 10 mg.

20. The method of any one of claims 1 to 19, wherein the SGLT-2 inhibitor, or a pharmaceutically acceptable salt or solvate thereof, is administered at a dose of about 8 mg.

21. The method of any one of claims 1 to 20, wherein the SGLT-2 inhibitor, or a pharmaceutically acceptable salt or solvate thereof, is administered at a dose of about 5 mg.

22. The method of any one of claims 1 to 21, wherein the SGLT-2 inhibitor, or a pharmaceutically acceptable salt or solvate thereof, is administered to the subject twice a day, daily, every other day, three times a week, twice a week, weekly, every other week, twice a month, or monthly.

23. The method of any one of claims 1 to 22, wherein the SGLT-2 inhibitor, or a pharmaceutically acceptable salt or solvate thereof, is administered to the subject daily.

24. The method of any one of claims 1 to 23, wherein the method further comprises administering (c) a GLP-1 receptor agonist.

25. The method of claim 24, wherein the GLP-1 receptor agonist is selected from the group consisting of: liraglutide, dulaglutide, exenatide, taspoglutide, lixisenatide, albiglutide, semaglutide, GLP-1, or a combination of two or more thereof.

26. The method of claim 24 or 25, wherein the GLP-1 receptor agonist is liraglutide.

27. A method of treating non-alcoholic fatty liver disease (NAFLD) in a subject in need thereof comprising administering to the subject
(a) the compound of Formula (I), 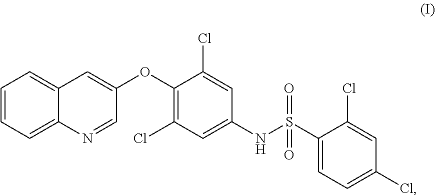 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) a GLP-1 receptor agonist, or a pharmaceutically acceptable salt or solvate thereof, wherein the amounts of (a) and (b) together are effective in treating NAFLD.

28. A method of treating a subject, the method comprising:
selecting a subject having non-alcoholic fatty liver disease (NAFLD); and
administering
(a) the compound of Formula (I), 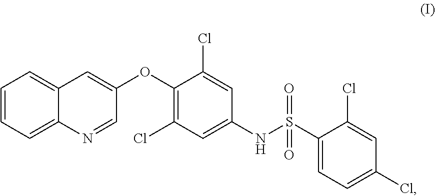 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) a GLP-1 receptor agonist, or a pharmaceutically acceptable salt or solvate thereof, to the selected subject
wherein the amounts of (a) and (b) together are effective in treating NAFLD.

29. A method of treating a subject, the method comprising:
identifying a subject having non-alcoholic fatty liver disease (NAFLD); and
administering
(a) the compound of Formula (I), 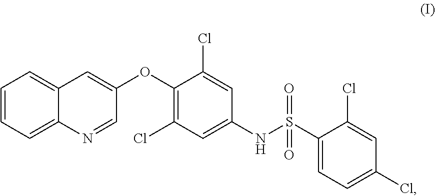 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) a GLP-1 receptor agonist, or a pharmaceutically acceptable salt or solvate thereof, to the selected subject
wherein the amounts of (a) and (b) together are effective in treating NAFLD.

30. A method of treating non-alcoholic fatty liver disease (NAFLD) in a subject in need thereof comprising administering to the subject
(a) a therapeutically effective amount of the compound of Formula (I), 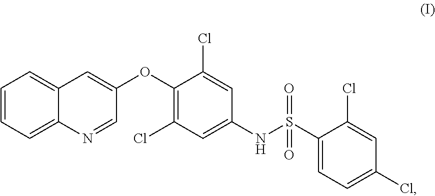 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) a therapeutically effective amount of a GLP-1 receptor agonist, or a pharmaceutically acceptable salt or solvate thereof.

31. A method of treating a subject, the method comprising:
selecting a subject having non-alcoholic fatty liver disease (NAFLD); and
administering
(a) a therapeutically effective amount of the compound of Formula (I), 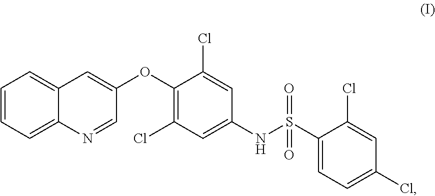 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) a therapeutically effective amount of a GLP-1 receptor agonist, or a pharmaceutically acceptable salt or solvate thereof, to the selected subject.

32. A method of treating fibrosis in a subject in need thereof comprising administering to the subject
(a) the compound of Formula (I), 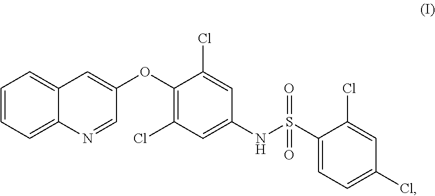 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) a GLP-1 receptor agonist, or a pharmaceutically acceptable salt or solvate thereof, wherein the amounts of (a) and (b) together are effective in treating fibrosis.

33. A method of treating fibrosis in a subject in need thereof comprising administering to the subject
(a) a therapeutically effective amount of the compound of Formula (I), 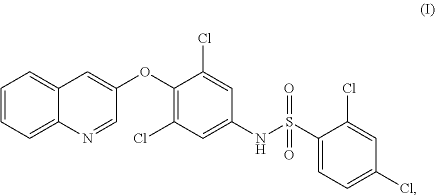 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) a therapeutically effective amount of a GLP-1 receptor agonist, or a pharmaceutically acceptable salt or solvate thereof.

34. The method of claims 32 or 33, wherein the fibrosis is cirrhosis.

35. The method of any one of claims 32 to 34, wherein the fibrosis is associated with NAFLD.

36. The method of any one of claims 32 to 35, wherein the fibrosis is caused by NAFLD.

37. The method of any one of claims 27 to 36, wherein the NAFLD is NASH.

38. The method of any one of claims 27 to 37, wherein the GLP-1 receptor agonist is selected from the group consisting of: liraglutide, dulaglutide, exenatide, taspoglutide, lixisenatide, albiglutide, semaglutide, GLP-1, or a combination of two or more thereof.

39. The method of any one of claims 27 to 38, wherein the GLP-1 receptor agonist is liraglutide.

40. The method of any one of claims 27 to 39, wherein (a) and (b) are administered concurrently.

41. The method of any one of claims 27 to 40, wherein (a) and (b) are administered sequentially in either order.

42. The method of any one of claims 27 to 41, wherein the GLP-1 receptor agonist, or a pharmaceutically acceptable salt or solvate thereof, is administered at a dose from about 0.1 to about 10 mg.

43. The method of any one of claims 27 to 42, wherein the GLP-1 receptor agonist, or a pharmaceutically acceptable salt or solvate thereof, is administered at a dose from about 0.1 to about 5 mg.

44. The method of any one of claims 27 to 43, wherein the GLP-1 receptor agonist, or a pharmaceutically acceptable salt or solvate thereof, is administered at a dose of about 1.0 mg.

45. The method of any one of claims 27 to 43, wherein the GLP-1 receptor agonist, or a pharmaceutically acceptable salt or solvate thereof, is administered at a dose of about 1.5 mg.

46. The method of any one of claims 27 to 43, wherein the GLP-1 receptor agonist, or a pharmaceutically acceptable salt or solvate thereof, is administered at a dose of about 1.8 mg.

47. The method of any one of claims 27 to 46, wherein the GLP-1 receptor agonist, or a pharmaceutically acceptable salt or solvate thereof, is administered to the subject twice a day, daily, every other day, three times a week, twice a week, weekly, every other week, twice a month, or monthly.

48. The method of any one of claims 27 to 47, wherein the GLP-1 receptor agonist, or a pharmaceutically acceptable salt or solvate thereof, is administered to the subject daily.

49. The method of any one of claims 27 to 47, wherein the GLP-1 receptor agonist, or a pharmaceutically acceptable salt or solvate thereof, is administered to the subject weekly.

50. The method of any one of claims 27 to 49, wherein the method further comprises administering (c) a SGLT-2 inhibitor.

51. The method of claim 50, wherein the SGLT-2 inhibitor is selected from the group consisting of: empagliflozin, canagliflozin, dapagliflozin, ertugliflozin, ipragliflozin, luseogliflozin, remogliflozin etabonate, serfliflozin etabonate, sotagliflozin, tofogliflozin, or a combination of two or more thereof.

52. The method of claim 50 or 51, wherein the SGLT-2 inhibitor is empagliflozin.

53. The method of claim 50 or 51, wherein the SGLT-2 inhibitor is dapagliflozin propylene glycol hydrate.

54. The method of any one of claims 1 to 5, 11 to 31, and 38 to 53, wherein the treatment of NAFLD comprises a reduction in hepatic steatosis.

55. The method of any one of claims 1 to 5, 11 to 31, and 38 to 54, wherein the treatment of NAFLD comprises a reduction in hepatic inflammation.

56. The method of any one of claims 1 to 5, 11 to 31, and 38 to 55, wherein the NAFLD activity score (NAS) following administration is 7 or less.

57. The method of any one of claims 1 to 5, 11 to 31, and 38 to 56, wherein the NAS is 5 or less.

58. The method of any one of claims 1 to 5, 11 to 31, and 38 to 57, wherein the NAS is 3 or less.

59. The method of any one of claims 1 to 5, 11 to 31, and 38 to 58, wherein the treatment of the NAFLD comprises treatment of liver fibrosis.

60. The method of any one of claims 1 to 5, 11 to 31, and 38 to 59, wherein the treatment of the NAFLD comprises treatment of liver cirrhosis.

61. The method of any one of claims 6 to 11, 32-37, and 58 to 60, wherein the treatment of fibrosis comprises a decrease in the stage of fibrosis, a lack of progression of the fibrosis, or a slowing in the progression of the fibrosis.

62. The method of any one of claims 6 to 11, 32-37, and 58 to 61, wherein the treatment of fibrosis comprises a decrease in the stage of fibrosis.

63. The method of any one of claims 6 to 11, 32-37, and 58 to 62, wherein the decrease in the stage of fibrosis is from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.

64. The method of any one of claims 1 to 5, 11 to 31, and 38 to 63, wherein the adiponectin level in the subject is increased by at least about 30%, at least about 68%, at least about 175%, or at least about 200%.

65. The method of any one of claims 1 to 5, 11 to 31, and 38 to 64, wherein the level of one or more biomarkers indicative of one or more of liver damage, inflammation, fibrosis, and/or cirrhosis is decreased.

66. The method of claim 65, wherein the increase is by at least about 175%.

67. The method of any one of claims 1 to 5, 11 to 31, and 38 to 66, wherein the treatment of NAFLD decreases the level of serum bile acids in the subject.

68. The method of any one of claims 1 to 5, 11 to 31, and 38 to 67, wherein the treatment of NAFLD comprises treatment of pruritus.

69. The method of any one of 1 to 5, 11 to 31, and 38 to 68, wherein the subject has liver fibrosis associated with the NAFLD.

70. The method of any one of claims 1 to 5, 11 to 31, and 38 to 69, wherein the subject has hepatic cirrhosis associated with the NAFLD.

71. The method of any one of claims 1 to 5, 11 to 31, and 38 to 70, wherein the subject has liver fibrosis as a comorbidity.

72. The method of any one of claims 1 to 5, 11 to 31, and 38 to 71, wherein the subject has hepatic cirrhosis as a comorbidity.

73. The method of any one of claims 1 to 5, 11 to 31, and 38 to 72, wherein the subject has liver fibrosis caused by the NAFLD.

74. The method of any one of claims 1 to 5, 11 to 31, and 38 to 73, wherein the subject has hepatic cirrhosis caused by the NAFLD.

75. The method of any one of claims 1 to 5, 11 to 31, and 38 to 74, wherein the NAFLD is simple nonalcoholic fatty liver (NAFL).

76. The method of any one of claims 1 to 5, 11 to 31, and 38 to 75, wherein the NAFLD is NAFL with attendant liver fibrosis.

77. The method of any one of claims 1 to 5, 11 to 31, and 38 to 76, wherein the NAFLD is NAFL with attendant liver cirrhosis.

78. The method of any one of claims 1 to 5, 11 to 31, and 38 to 77, wherein the treatment of NAFL decreases the level of serum bile acids in the subject.

79. The method of any one of claims 1 to 5, 11 to 31, and 38 to 78, wherein the treatment of NAFL comprises treatment of pruritus.

80. The method of any one of claims 1 to 5, 11 to 31, and 38 to 79, wherein the NAFLD is nonalcoholic steatohepatitis (NASH).

81. The method of any one of claims 1 to 5, 11 to 31, 38 to 74 and 80, wherein the NAFLD is NASH with attendant liver fibrosis.

82. The method of any one of claims 1 to 5, 11 to 31, 38 to 74, 79, and 81, wherein the NAFLD is NASH with attendant liver cirrhosis.

83. The method of any one of claims 1 to 5, 11 to 31, 38 to 74, and 79 to 81, wherein the treatment of NASH decreases the level of serum bile acids in the subject.

84. The method of any one of claims 1 to 5, 11 to 31, 38 to 74, and 79 to 83, wherein the treatment of NASH comprises treatment of pruritus.

85. A method of treating non-alcoholic fatty liver disease (NAFLD) in a subject in need thereof comprising administering to the subject
(a) the compound of Formula (I), 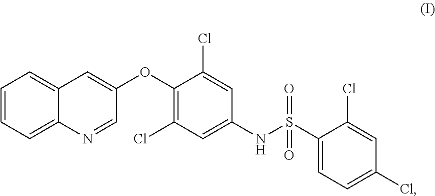 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
(c) a GLP-1 receptor agonist, or a pharmaceutically acceptable salt or solvate thereof, and
(d) metformin, or a pharmaceutically acceptable salt thereof;
wherein the amounts of (a), (b), (c), and (d) together are effective in treating NAFLD.

86. A method of treating a subject, the method comprising:
selecting a subject having non-alcoholic fatty liver disease (NAFLD); and
administering
(a) the compound of Formula (I), 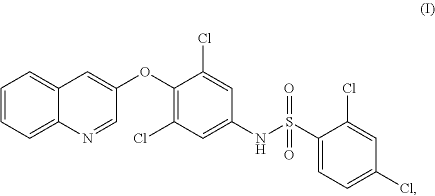 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
(c) a GLP-1 receptor agonist, or a pharmaceutically acceptable salt or solvate thereof, and
(d) metformin, or a pharmaceutically acceptable salt thereof;
wherein the amounts of (a), (b), (c), and (d) together are effective in treating NAFLD.

87. A method of treating a subject, the method comprising:
identifying a subject having non-alcoholic fatty liver disease (NAFLD); and
administering
(a) the compound of Formula (I), 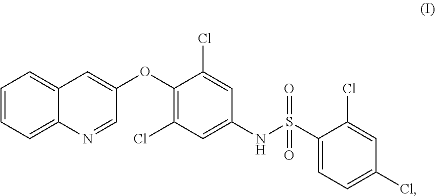 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
(c) a GLP-1 receptor agonist, or a pharmaceutically acceptable salt or solvate thereof, and
(d) metformin, or a pharmaceutically acceptable salt thereof;
wherein the amounts of (a), (b), (c), and (d) together are effective in treating NAFLD.

88. A method of treating non-alcoholic fatty liver disease (NAFLD) in a subject in need thereof comprising administering to the subject
(a) a therapeutically effective amount of the compound of Formula (I), 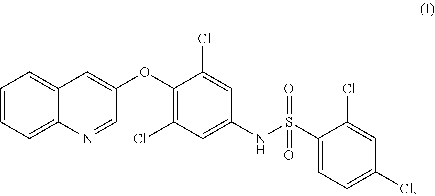 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
(c) a GLP-1 receptor agonist, or a pharmaceutically acceptable salt or solvate thereof, and
(d) metformin, or a pharmaceutically acceptable salt thereof;
wherein the amounts of (a), (b), (c), and (d) together are effective in treating NAFLD.

89. A method of treating a subject, the method comprising:
selecting a subject having non-alcoholic fatty liver disease (NAFLD); and
administering
(a) a therapeutically effective amount of the compound of Formula (I), 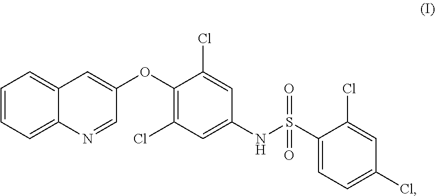 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
(c) a GLP-1 receptor agonist, or a pharmaceutically acceptable salt or solvate thereof, and
(d) metformin, or a pharmaceutically acceptable salt thereof;
wherein the amounts of (a), (b), (c), and (d) together are effective in treating NAFLD.

90. A method of treating fibrosis in a subject in need thereof comprising administering to the subject
(a) the compound of Formula (I), 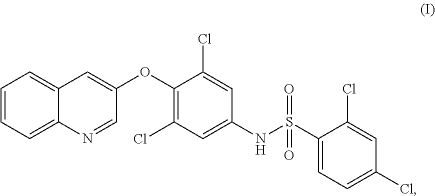 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
(c) a GLP-1 receptor agonist, or a pharmaceutically acceptable salt or solvate thereof, and
(d) metformin, or a pharmaceutically acceptable salt thereof;
wherein the amounts of (a), (b), (c), and (d) together are effective in treating fibrosis.

91. A method of treating fibrosis in a subject in need thereof comprising administering to the subject
(a) a therapeutically effective amount of the compound of Formula (I), 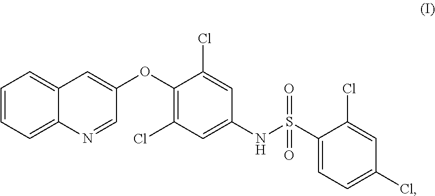 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
(c) a GLP-1 receptor agonist, or a pharmaceutically acceptable salt or solvate thereof, and
(d) metformin, or a pharmaceutically acceptable salt thereof;
wherein the amounts of (a), (b), (c), and (d) together are effective in treating fibrosis.

92. A method of treating non-alcoholic fatty liver disease (NAFLD) in a subject in need thereof comprising administering to the subject
(a) the compound of Formula (I), 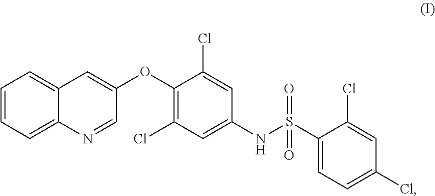 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
(d) metformin, or a pharmaceutically acceptable salt thereof;
wherein the amounts of (a), (b), and (d) together are effective in treating NAFLD.

93. A method of treating a subject, the method comprising:
selecting a subject having non-alcoholic fatty liver disease (NAFLD); and
administering
(a) the compound of Formula (I), 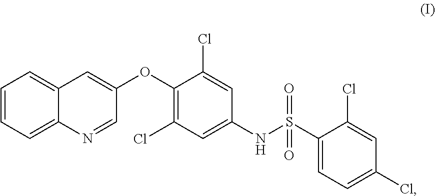 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
(d) metformin, or a pharmaceutically acceptable salt thereof;
wherein the amounts of (a), (b), and (d) together are effective in treating NAFLD.

94. A method of treating a subject, the method comprising:
identifying a subject having non-alcoholic fatty liver disease (NAFLD); and
administering
(a) the compound of Formula (I), 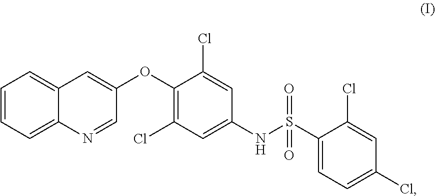 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
(d) metformin, or a pharmaceutically acceptable salt thereof;
wherein the amounts of (a), (b), and (d) together are effective in treating NAFLD.

95. A method of treating non-alcoholic fatty liver disease (NAFLD) in a subject in need thereof comprising administering to the subject
(a) a therapeutically effective amount of the compound of Formula (I), 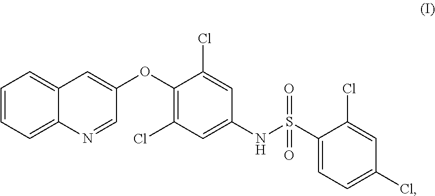 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
(d) metformin, or a pharmaceutically acceptable salt thereof;
wherein the amounts of (a), (b), and (d) together are effective in treating NAFLD.

96. A method of treating a subject, the method comprising:
selecting a subject having non-alcoholic fatty liver disease (NAFLD); and
administering
(a) a therapeutically effective amount of the compound of Formula (I), 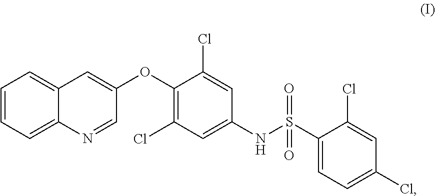 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
(d) metformin, or a pharmaceutically acceptable salt thereof;
wherein the amounts of (a), (b), and (d) together are effective in treating NAFLD.

97. A method of treating fibrosis in a subject in need thereof comprising administering to the subject
(a) the compound of Formula (I), 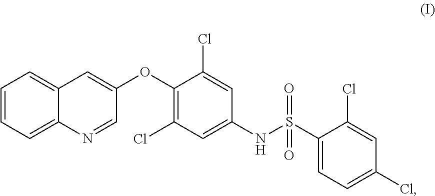 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
(d) metformin, or a pharmaceutically acceptable salt thereof;
wherein the amounts of (a), (b), and (d) together are effective in treating fibrosis.

98. A method of treating fibrosis in a subject in need thereof comprising administering to the subject
(a) a therapeutically effective amount of the compound of Formula (I), 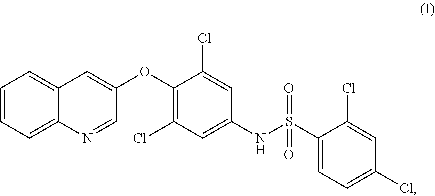 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
(d) metformin, or a pharmaceutically acceptable salt thereof;
wherein the amounts of (a), (b), and (d) together are effective in treating fibrosis.

99. A method of treating non-alcoholic fatty liver disease (NAFLD) in a subject in need thereof comprising administering to the subject
(a) the compound of Formula (I), 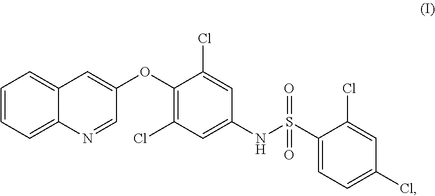 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(c) a GLP-1 receptor agonist, or a pharmaceutically acceptable salt or solvate thereof, and
(d) metformin, or a pharmaceutically acceptable salt thereof;
wherein the amounts of (a), (c), and (d) together are effective in treating NAFLD.

100. A method of treating a subject, the method comprising:
selecting a subject having non-alcoholic fatty liver disease (NAFLD); and
administering
(a) the compound of Formula (I), 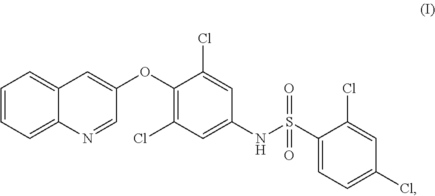 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(c) a GLP-1 receptor agonist, or a pharmaceutically acceptable salt or solvate thereof, and
(d) metformin, or a pharmaceutically acceptable salt thereof;
wherein the amounts of (a), (c), and (d) together are effective in treating NAFLD.

101. A method of treating a subject, the method comprising:
identifying a subject having non-alcoholic fatty liver disease (NAFLD); and
administering
(a) the compound of Formula (I), 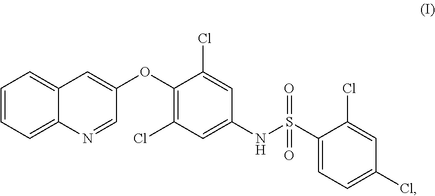 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(c) a GLP-1 receptor agonist, or a pharmaceutically acceptable salt or solvate thereof, and
(d) metformin, or a pharmaceutically acceptable salt thereof;
wherein the amounts of (a), (c), and (d) together are effective in treating NAFLD.

102. A method of treating non-alcoholic fatty liver disease (NAFLD) in a subject in need thereof comprising administering to the subject
(a) a therapeutically effective amount of the compound of Formula (I), 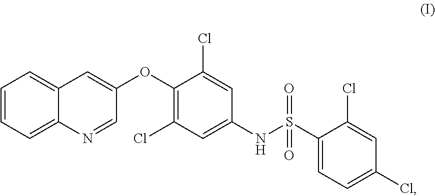 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(c) a GLP-1 receptor agonist, or a pharmaceutically acceptable salt or solvate thereof, and
(d) metformin, or a pharmaceutically acceptable salt thereof;
wherein the amounts of (a), (c), and (d) together are effective in treating NAFLD.

103. A method of treating a subject, the method comprising:
selecting a subject having non-alcoholic fatty liver disease (NAFLD); and
administering
(a) a therapeutically effective amount of the compound of Formula (I), 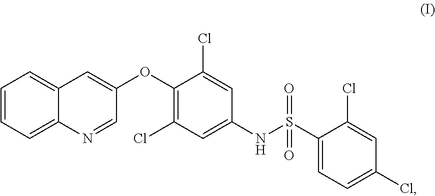 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(c) a GLP-1 receptor agonist, or a pharmaceutically acceptable salt or solvate thereof, and
(d) metformin, or a pharmaceutically acceptable salt thereof;
wherein the amounts of (a), (c), and (d) together are effective in treating NAFLD.

104. A method of treating fibrosis in a subject in need thereof comprising administering to the subject
(a) the compound of Formula (I), 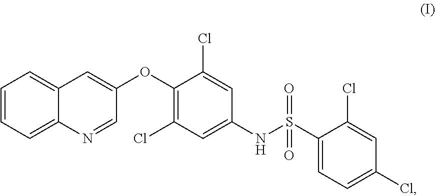 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(c) GLP-1 receptor agonist, or a pharmaceutically acceptable salt or solvate thereof, and
(d) metformin, or a pharmaceutically acceptable salt thereof;
wherein the amounts of (a), (c), and (d) together are effective in treating fibrosis.

105. A method of treating fibrosis in a subject in need thereof comprising administering to the subject
(a) a therapeutically effective amount of the compound of Formula (I), 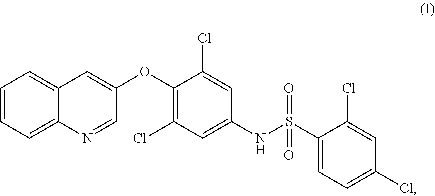 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(c) a GLP-1 receptor agonist, or a pharmaceutically acceptable salt or solvate thereof, and
(d) metformin, or a pharmaceutically acceptable salt thereof;
wherein the amounts of (a), (c), and (d) together are effective in treating fibrosis.

106. The method of claims 90, 91, 97, 98, 104, or 105, wherein the fibrosis is cirrhosis.

107. The method of any one of claims 90, 91, 97, 98, 104, or 105-106, wherein the fibrosis is associated with NAFLD.

108. The method of any one of claims 90, 91, 97, 98, 104, or 105-107, wherein the fibrosis is caused by NAFLD.

109. The method of any one of claims 85-89, 92-96, or 99-103 wherein the NAFLD is NASH.

110. The method of any one of claims 85-98, wherein the SGLT-2 inhibitor is selected from the group consisting of: empagliflozin, canagliflozin, dapagliflozin, ertugliflozin, ipragliflozin, luseogliflozin, remogliflozin etabonate, serfliflozin etabonate, sotagliflozin, tofogliflozin, or a combination of two or more thereof.

111. The method of any one of claims 85-98 or 110, wherein the SGLT-2 inhibitor is empagliflozin.

112. The method of any one of claims 85-98 or 110-111, wherein the SGLT-2 inhibitor is dapagliflozin propylene glycol hydrate.

113. The method of any one of claims 85-98 or 110-112, wherein the SGLT-2 inhibitor, or a pharmaceutically acceptable salt or solvate thereof, is administered at a dose from about 1 to about 350 mg.

114. The method of any one of claims 85-98 or 110-113, wherein the SGLT-2 inhibitor, or a pharmaceutically acceptable salt or solvate thereof, is administered at a dose from about 85 to about 325 mg.

115. The method of any one of claims 85-98 or 110-114, wherein the SGLT-2 inhibitor, or a pharmaceutically acceptable salt or solvate thereof, is administered at a dose from about 5 to about 15 mg.

116. method of any one of claims 85-98 or 110-115, wherein the SGLT-2 inhibitor, or a pharmaceutically acceptable salt or solvate thereof, is administered at a dose of about 10 mg.

117. The method of any one of claims 85-98 or 110-116, wherein the SGLT-2 inhibitor, or a pharmaceutically acceptable salt or solvate thereof, is administered at a dose of about 8 mg.

118. The method of any one of claims 85-98 or 110-117, wherein the SGLT-2 inhibitor, or a pharmaceutically acceptable salt or solvate thereof, is administered at a dose of about 5 mg.

119. The method of any one of claims 85-91 or 99-105, wherein the GLP-1 receptor agonist is selected from the group consisting of: liraglutide, dulaglutide, exenatide, taspoglutide, lixisenatide, albiglutide, semaglutide, GLP-1, or a combination of two or more thereof.

120. The method of any one of claims 85-91, 99-105 or 119, wherein the GLP-1 receptor agonist is liraglutide.

121. The method of any one of claims 85-91, 99-105 or 119-120, wherein the GLP-1 receptor agonist, or a pharmaceutically acceptable salt or solvate thereof, is administered at a dose from about 0.1 to about 10 mg.

122. The method of any one of claims 85-91, 99-105 or 119-121, wherein the GLP-1 receptor agonist, or a pharmaceutically acceptable salt or solvate thereof, is administered at a dose from about 0.1 to about 5 mg.

123. method of any one of claims 85-91, 99-105 or 119-123, wherein the GLP-1 receptor agonist, or a pharmaceutically acceptable salt or solvate thereof, is administered at a dose of about 1.0 mg.

124. The method of any one of claims 85-91, 99-105 or 119-123, wherein the GLP-1 receptor agonist, or a pharmaceutically acceptable salt or solvate thereof, is administered at a dose of about 1.5 mg.

125. The method of any one of claims 85-91, 99-105 or 119-123, wherein the GLP-1 receptor agonist, or a pharmaceutically acceptable salt or solvate thereof, is administered at a dose of about 1.8 mg.

126. The method of any one of claims 85-125, wherein the metformin, or a pharmaceutically acceptable salt thereof, is administered at a dose of from about 250 to about 2,500 mg.

127. The method of any one of claims 85-126, wherein the metformin, or a pharmaceutically acceptable salt thereof, is administered at a dose of from about 500 to about 2,000 mg.

128. The method of any one of claims 85-127, wherein the metformin, or a pharmaceutically acceptable salt thereof, is administered at a dose of from about 750 to about 1,500 mg.

129. The method of any one of claims 85-128, wherein the metformin, or a pharmaceutically acceptable salt thereof, is administered at a dose of from about 1,000 to about 1,250 mg.

130. The method of any one of claims 85-91 or 110-129, wherein (a), (b), (c), and (d) are administered concurrently.

131. The method of any one of claims 85-91 or 110-129, wherein (a), (b), (c), and (d) are administered sequentially in any order.

132. The method of any one of claims 92-98 or 110-129, wherein (a), (b), and (d) are administered concurrently.

133. The method of any one of claims 92-98 or 110-129, wherein (a), (b), and (d) are administered sequentially in any order.

134. The method of any one of claims 99-105 or 110-129, wherein (a), (c), and (d) are administered concurrently.

135. The method of any one of claims 99-105 or 110-129, wherein (a), (c), and (d) are administered sequentially in any order.

136. The method of any one of claims 1 to 135, wherein the compound of Formula (I), a pharmaceutically acceptable salt or solvate thereof, is administered prophylactically.

137. The method of any one of claims 1 to 136, wherein the compound of Formula (I), or a pharmaceutically acceptable salt or solvate thereof, is administered at a dose from about 0.1 to about 15 mg.

138. The method of any one of claims 1 to 137, wherein the compound of Formula (I), or a pharmaceutically acceptable salt or solvate thereof, is administered at a dose from about 1 to about 10 mg.

139. The method of any one of claims 1 to 138, wherein the compound of Formula (I), or a pharmaceutically acceptable salt or solvate thereof, is administered at a dose from about 2 to about 6 mg.

140. The method of any one of claims 1 to 139, wherein the compound of Formula (I), or a pharmaceutically acceptable salt or solvate thereof, is administered at a dose from about 0.5 to about 3 mg.

141. The method of any one of claims 1 to 140, wherein the compound of Formula (I), or a pharmaceutically acceptable salt or solvate thereof, is administered at a dose of about 3 mg.

142. The method of any one of claims 1 to 141, wherein the compound of Formula (I), or a pharmaceutically acceptable salt or solvate thereof, is administered at a dose of about 2 mg.

143. The method of any one of claims 1 to 142, wherein the compound of Formula (I), or a pharmaceutically acceptable salt or solvate thereof, is administered at a dose of about 1 mg.

144. The method of any one of claims 1 to 143, wherein the compound of Formula (I), or a pharmaceutically acceptable salt or solvate thereof, is administered to the subject twice a day, daily, every other day, three times a week, twice a week, weekly, every other week, twice a month, or monthly.

145. The method of any one of claims 1 to 144, wherein the compound of Formula (I), or a pharmaceutically acceptable salt or solvate thereof, is administered to the subject daily.

146. The method of any one of claims 1 to 145, wherein the compound of Formula (I), or a pharmaceutically acceptable salt or solvate thereof, is administered to the subject daily and the dose of the compound of Formula (I) is about 3 mg.

147. The method of any one of claims 1 to 146, wherein the compound of Formula (I), or a pharmaceutically acceptable salt or solvate thereof, is administered at a dose from about 0.1 to about 10.0 mg per day.

148. The method of any one of claims 1 to 147, wherein the compound of Formula (I), or a pharmaceutically acceptable salt or solvate thereof, is administered at a dose from about 0.1 to about 3 mg per day.

149. The method of any one of claims 1 to 148, wherein the compound of Formula (I), or a pharmaceutically acceptable salt or solvate thereof, is administered at a dose of about 0.5 milligram per day.

150. The method of any one of claims 1 to 148, wherein the compound of Formula (I), or a pharmaceutically acceptable salt or solvate thereof, is administered at a dose of about 1 milligram per day.

151. The method of any one of claims 1 to 148, wherein the compound of Formula (I), or a pharmaceutically acceptable salt or solvate thereof, is administered at a dose of about 2 mg per day.

152. The method of any one of claims 1 to 151, wherein the compound of Formula (I) is in the form of a besylate salt.

153. The method of any one of claims 1 to 152, wherein the method further comprises performing a liver biopsy to determine the NAFLD activity score of the biopsy sample obtained from the subject.

154. A method of treating non-alcoholic fatty liver disease (NAFLD) in a subject in need thereof consisting essentially of administering to the subject
(a) the compound of Formula (I), 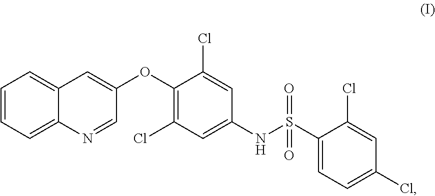 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof, wherein the amounts of (a) and (b) together are effective in treating NAFLD.

155. A method of treating non-alcoholic fatty liver disease (NAFLD) in a subject in need thereof consisting essentially of administering to the subject
(a) the compound of Formula (I), 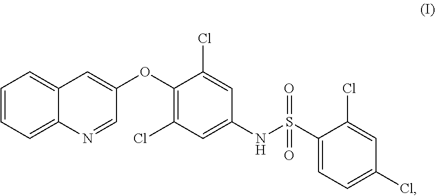 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an GLP-1 receptor agonist, or a pharmaceutically acceptable salt or solvate thereof,
wherein the amounts of (a) and (b) together are effective in treating NAFLD.

156. A method of treating non-alcoholic fatty liver disease (NAFLD) in a subject in need thereof consisting essentially of administering to the subject
(a) the compound of Formula (I), 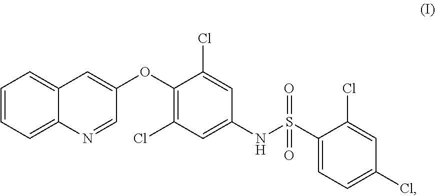 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof,
(b) an SGLT-2 inhibitor, or a pharmaceutically acceptable salt, salt or solvate thereof, and
(c) an GLP-1 receptor agonist, or a pharmaceutically acceptable salt or solvate thereof,
wherein the amounts of (a), (b), and (c) together are effective in treating NAFLD.

157. A method of treating non-alcoholic fatty liver disease (NAFLD) in a subject in need thereof consisting essentially of administering to the subject
(a) the compound of Formula (I), 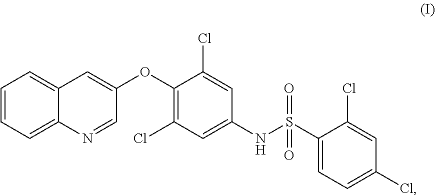 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof,
(b) an SGLT-2 inhibitor, or a pharmaceutically acceptable salt or solvate thereof,
(c) an GLP-1 receptor agonist, or a pharmaceutically acceptable salt or solvate thereof, and
(d) metformin, or a pharmaceutically acceptable salt thereof,
wherein the amounts of (a), (b), (c), and (d) together are effective in treating NAFLD.

158. A pharmaceutical composition comprising
(a) the compound of Formula (I), 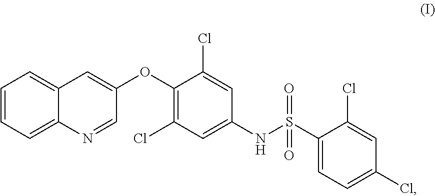 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof,
(b) an SGLT-2 inhibitor, or a pharmaceutically acceptable salt or solvate thereof, and
one or more pharmaceutical excipients, wherein the amounts of (a) and (b) together are effective in treating NAFLD.

159. The pharmaceutical composition of claim 158, wherein the composition further comprises metformin, or a pharmaceutically acceptable salt thereof.

160. A pharmaceutical composition comprising
(a) the compound of Formula (I), 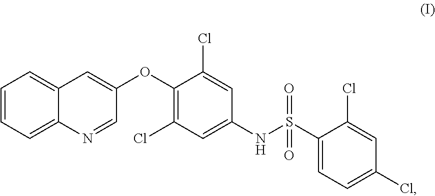 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof,
(b) a GLP-1 receptor agonist, or a pharmaceutically acceptable salt or solvate thereof, and one or more pharmaceutical excipients,
wherein the amounts of (a) and (b) together are effective in treating NAFLD.

161. The pharmaceutical composition of claim 160, wherein the composition further comprises metformin, or a pharmaceutically acceptable salt thereof.

162. A pharmaceutical composition comprising
(a) the compound of Formula (I), 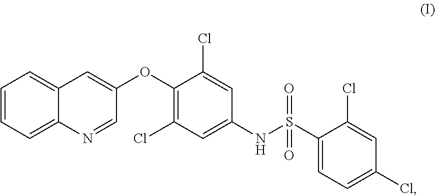 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof,
(b) an SGLT-2 inhibitor, or a pharmaceutically acceptable salt or solvate thereof,
(c) a GLP-1 receptor agonist, or a pharmaceutically acceptable salt or solvate thereof, and
one or more pharmaceutical excipients, wherein the amounts of (a) and (b) together are effective in treating NAFLD.

163. The pharmaceutical composition of claim 162, wherein the composition further comprises metformin, or a pharmaceutically acceptable salt thereof.

164. A pharmaceutical combination comprising
(a) the compound of Formula (I), 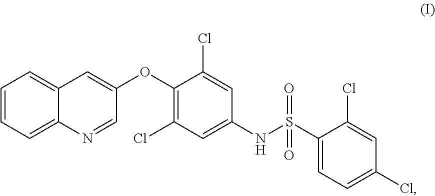 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) an SGLT-2 inhibitor, or a pharmaceutically acceptable salt or solvate thereof,
for concurrent or sequential administration for use in the treatment of non-alcoholic fatty liver disease (NAFLD).

165. The pharmaceutical combination of claim 164, wherein the composition further comprises metformin, or a pharmaceutically acceptable salt thereof.

166. A pharmaceutical combination comprising
(a) the compound of Formula (I), 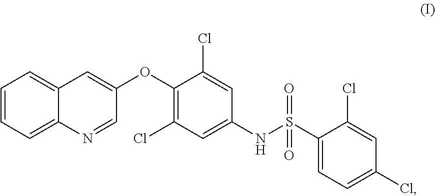 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) a GLP-1 receptor agonist, or a pharmaceutically acceptable salt or solvate thereof,
for concurrent or sequential administration for use in the treatment of non-alcoholic fatty liver disease (NAFLD).

167. The pharmaceutical combination of claim 166, wherein the composition further comprises metformin, or a pharmaceutically acceptable salt thereof.

168. A pharmaceutical combination of any one of claims 164-167, further comprising at least one pharmaceutically acceptable carrier.
